(19) United States
(12) Patent Application Publication
(10) Pub. No.: US 2018/0251557 A1
(43) Pub. Date: Sep. 6, 2018

(54) METHODS OF TREATING HER2-POSITIVE CANCER

(71) Applicant: GENENTECH, INC., South San Francisco, CA (US)

(72) Inventors: Stephen Chui, South San Francisco, CA (US); Melanie Smitt, South San Francisco, CA (US); Monika Patre, South San Francisco, CA (US)

(73) Assignee: GENENTECH, INC., South San Francisco, CA (US)

(21) Appl. No.: 15/980,519

(22) Filed: May 15, 2018

Related U.S. Application Data
(63) Continuation of application No. PCT/US2016/061644, filed on Nov. 11, 2016.
(60) Provisional application No. 62/379,143, filed on Aug. 24, 2016.
(60) Provisional application No. 62/256,091, filed on Nov. 16, 2015.

(51) Int. Cl. C07K 16/28; C07K 16/32; A61P 31/00; A61P 35/00; A61K 47/68; A61K 39/395; A61K 31/5365

(52) U.S. Cl. CPC C07K 16/2827; C07K 16/32; A61P 31/00; A61P 35/00; A61K 2039/507; A61K 39/39558; A61K 31/5365; C07K 2317/24; C07K 2317/56; A61K 47/6803

(57) ABSTRACT

Methods of treating patients having HER2-positive cancer are provided. Certain methods involve treatment of HER2 positive breast cancer using a programmed cell death protein 1 (PD-1) binding antagonist or a programmed death ligand 1 (PD-L1) binding antagonist in combination with trastuzumab and pertuzumab or with trastuzumab emtansine. The treatment regimen may be used in various clinical settings, for example, for treatment in the neoadjuvant or metastatic setting.

### FIELD OF THE INVENTION

The invention relates to methods of using a Programmed cell death protein 1 (PD-1) binding antagonist or a programmed death ligand 1 (PD-L1) binding antagonist, in combination with a HER2-targeted therapy, for the treatment of HER2 positive cancer.

The instant application contains a Sequence Listing which has been submitted electronically in ASCII format and is hereby incorporated by reference in its entirety. Said ASCII copy, created on Nov. 2, 2016, is named GNE0424 SL.txt and is 19,829 bytes in size.

### BACKGROUND OF THE INVENTION

Breast Cancer and HER2 Targeted Treatments

The HER2 (ErbB2) receptor tyrosine kinase is a member of the epidermal growth factor receptor (EGFR) family of transmembrane receptors. Overexpression of HER2 is observed in approximately 20% of human breast cancers (hereinafter referred to as HER2-positive breast cancer) and is implicated in the aggressive growth and poor clinical outcomes associated with these tumors (Slamon et al (1987) Science 235:177-182). HER2 protein overexpression can be determined using an immunohistochemistry based assessment of fixed tumor blocks (Press M F, et al (1993) Cancer Res 53:4960-70).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. No. 5,677,171; U.S. Pat. No. 5,821,337; U.S. Pat. No. 6,054,297; U.S. Pat. No. 6,165,464; U.S. Pat. No. 6,339,142; U.S. Pat. No. 6,407,213; U.S. Pat. No. 6,639,055; U.S. Pat. No. 6,719,971; U.S. Pat. No. 6,800,738; U.S. Pat. No. 7,074,404; Coussens et al (1985) Science 230:1132-9; Slamon et al (1989) Science 244:707-12; Slamon et al (2001) New Engl. J. Med. 344:783-792). Trastuzumab has been shown, in both in vitro assays and in animals, to inhibit the proliferation of human tumor cells that overexpress HER2 (Hudziak et al (1989) Mol Cell Biol 9:1165-72; Lewis et al (1993) Cancer Immunol Immunother; 37:255-63; Baselga et al (1998) Cancer Res. 58:2825-2831). Trastuzumab is a mediator of antibody-dependent cellular cytotoxicity, ADCC (Lewis et al (1993) Cancer Immunol Immunother 37(4):255-263; Hotaling et al (1996) [abstract]. Proc. Annual Meeting Am Assoc Cancer Res; 37:471; Pegram M D, et al (1997) [abstract]. Proc Am Assoc Cancer Res; 38:602; Sliwkowski et al (1999) Seminars in Oncology 26(4), Suppl 12:60-70; Yarden Y. and Sliwkowski, M. (2001) Nature Reviews: Molecular Cell Biology, Macmillan Magazines, Ltd., Vol. 2:127-137).

HERCEPTIN® (trastuzumab, Genentech Inc.) was approved in 1998 for the treatment of patients with HER2-overexpressing metastatic breast cancers (Baselga et al, (1996) J. Clin. Oncol. 14:737-744) that have received extensive prior anti-cancer therapy, and has since been used in over 300,000 patients (Slamon D J, et al. N Engl J Med 2001; 344:783-92; Vogel C L, et al. J Clin Oncol 2002; 20:719-26; Marty M, et al. J Clin Oncol 2005; 23:4265-74; Romond E H, et al. T N Engl J Med 2005; 353:1673-84; Piccart-Gebhart M J, et al. N Engl J Med 2005; 353:1659-72; Slamon D, et al. [abstract]. Breast Cancer Res Treat 2006, 100 (Suppl 1): 52). In 2006, the FDA approved HERCEPTIN® as part of a treatment regimen containing doxorubicin, cyclophosphamide and paclitaxel for the adjuvant treatment of patients with HER2-positive, node-positive breast cancer.

An alternative approach to antibody-targeted therapy is to utilize antibodies for delivery of cytotoxic drugs specifically to antigen-expressing cancer cells. Antibody-drug conjugates, or ADCs, are monoclonal antibodies to which highly potent cytotoxic agents have been conjugated. ADCs represent a novel approach to conferring tumor selectivity on systemically administered anti-tumor therapeutics. Utilizing surface antigens that are tumor-specific and/or overexpressed, ADCs are designed to focus the delivery of highly potent cytotoxic agents to tumor cells. The potential of this approach is to create a more favorable therapeutic window for such agents than could be achieved by their administration as free drugs. Maytansinoids, derivatives of the anti-mitotic drug maytansine, bind to microtubules in a manner similar to vinca alkaloid drugs (Issell B F et al (1978) Cancer Treat. Rev. 5:199-207; Cabanillas F et al. (1979) Cancer Treat Rep, 63:507-9. DM1 is a thiol-containing maytansinoid derived from the naturally occurring ester ansamitocin P3 (Remillard S, Rebhun L I, Howie G A, et al. (1975) Science 189(4207):1002-1005.3; Cassady J M, Chan K K, Floss H G. (2004) Chem Pharm Bull 52(1):1-26.4). The related plant ester, maytansine, has been studied as a chemotherapeutic agent in approximately 800 patients, administered at a dose of 2.0 mg/m2 every 3 weeks either as a single dose or for 3 consecutive days (Issell B F, Crooke S T. (1978) Maytansine. Cancer Treat Rev 5:199-207). Despite preclinical activity, the activity of maytansine in the clinic was modest at doses that could be safely delivered. The dose-limiting toxicity (DLT) was gastrointestinal, consisting of nausea, vomiting, and diarrhea (often followed by constipation). These toxicities were dose dependent but not schedule dependent. Peripheral neuropathy (predominantly sensory) was reported and was most apparent in patients with preexisting neuropathy. Subclinical transient elevations of hepatic transaminase, alkaline phosphatase, and total bilirubin were reported. Constitutional toxicities, including weakness, lethargy, dysphoria, and insomnia, were common. Less common toxicities included infusion-site phlebitis and mild myelosuppression. Further development of the drug was abandoned in the 1980s because of the narrow therapeutic window.

Trastuzumab-MCC-DM1 (T-DM1, trastuzumab emtansine, ado-trastuzumab emtansine, KADCYLA®), a novel antibody-drug conjugate (ADC) for the treatment of HER2-positive breast cancer, is composed of the cytotoxic agent DM1 (a thiol-containing maytansinoid anti-microtubule agent) conjugated to trastuzumab at lysine side chains via an MCC linker, with an average drug load (drug to antibody ratio) of about 3.5. After binding to HER2 expressed on tumor cells, T-DM1 undergoes receptor-mediated internalization, resulting in intracellular release of cytotoxic catabolites containing DM1 and subsequent cell death.

In a Phase I study of T-DM1 (TDM3569g), the maximum tolerated dose (MTD) of T-DM1 administered by IV infusion every 3 weeks (q3w) was 3.6 mg/kg. A DLT (Dose-Limiting Toxicity) consisted of transient thrombocytopenia in patients treated at 4.8 mg/kg. Treatment with 3.6 mg/kg q3w was well tolerated and associated with significant clinical activity. (Krop (2010) J. Clin. Oncol. 28(16):2698-2704). That same study also showed that weekly dosing with 2.4 mg/kg was also well tolerated and had anti-tumor activity. (Beeram (2012) Cancer 118(23):5733-5740.)

A Phase II study (TDM4374g) demonstrated that T-DM1, administered at 3.6 mg/kg q3w, had single-agent anti-tumor activity in a heavily pre-treated patient population having HER2-positive metastatic breast cancer. (Krop (2012) 30(26):3234-3241.) A Phase III study (TDM4370g, “EMILIA”) demonstrated that T-DM1, administered at 3.6 mg/kg q3w, significantly prolonged progression-free survival and overall survival with less toxicity compared to treatment with lapatinib plus capecitabine in patients with HER2-positive advanced breast cancer (2^{nd} and 3^{rd} line metastatic breast cancer) previously treated with trastuzumab and a taxane. (Verma (2012) New England Journal of Medicine 367:1783-1791.)

The U.S. Food and Drug Administration approved ado-trastuzumab emtansine, marketed under the tradename KADCYLA®, on Feb. 22, 2013 for the treatment of patients with HER2-positive, metastatic breast cancer who previously received treatment with trastuzumab and a taxane.

Pertuzumab (also known as recombinant humanized monoclonal antibody 2C4, rhuMAb 2C4, PERJETA®, Genentech, Inc, South San Francisco) represents the first in a new class of agents known as HER dimerization inhibitors (HDI) and functions to inhibit the ability of HER2 to form active heterodimers or homodimers with other HER receptors (such as EGFR/HER1, HER2, HER3 and HER4). See, for example, Harari and Yarden Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003)

Pertuzumab blockade of the formation of HER2-HER3 heterodimers in tumor cells has been demonstrated to inhibit critical cell signaling, which results in reduced tumor proliferation and survival (Agus et al. Cancer Cell 2:127-37 (2002)).

Pertuzumab has undergone testing as a single agent in the clinic with a phase Ia trial in patients with advanced cancers and phase II trials in patients with ovarian cancer and breast cancer as well as lung and prostate cancer. In a Phase I study, patients with incurable, locally advanced, recurrent or metastatic solid tumors that had progressed during or after standard therapy were treated with pertuzumab given intravenously every 3 weeks. Pertuzumab was generally well tolerated. Tumor regression was achieved in 3 of 20 patients evaluable for response. Two patients had confirmed partial responses. Stable disease lasting for more than 2.5 months was observed in 6 of 21 patients (Agus et al. Pro Am Soc Clin Oncol 22:192 (2003)). At doses of 2.0-15 mg/kg, the pharmacokinetics of pertuzumab was linear, and mean clearance ranged from 2.69 to 3.74 mL/day/kg and the mean terminal elimination half-life ranged from 15.3 to 27.6 days. Antibodies to pertuzumab were not detected (Allison et al. Pro Am Soc Clin Oncol 22:197 (2003)).

US 2006/0034842 describes methods for treating ErbB-expressing cancer with anti-ErbB2 antibody combinations. US 2008/0102069 describes the use of Trastuzumab and Pertuzumab in the treatment of HER2-positive metastatic cancer, such as breast cancer. Baselga et al., J Clin Oncol, 2007 ASCO Annual Meeting Proceedings Part I, Col. 25, No. 18S (June 20 Supplement), 2007:1004 report the treatment of patients with pre-treated HER2-positive breast cancer, which has progressed during treatment with Trastuzumab, with a combination of Trastuzumab and Pertuzumab. Portera et al., J Clin Oncol, 2007 ASCO Annual Meeting Proceedings Part I. Vol. 25, No. 18S (June 20 Supplement), 2007:1028 evaluated the efficacy and safety of Trastuzumab+Pertuzumab combination therapy in HER2-positive breast cancer patients, who had progressive disease on Trastuzumab-based therapy. The authors concluded that further evaluation of the efficacy of combination treatment was required to define the overall risk and benefit of this treatment regimen.

Pertuzumab has been evaluated in Phase II studies in combination with Trastuzumab in patients with HER2-positive metastatic breast cancer who have previously received Trastuzumab for metastatic disease. One study, conducted by the National cancer Institute (NCl), enrolled 11 patients with previously treated HER2-positive metastatic breast cancer. Two out of the 11 patients exhibited a partial response (PR) (Baselga et al., J Clin Oncol 2007 ASCO Annual Meeting Proceedings; 25:18 S (June 20 Supplement): 1004. The results of a Phase II neoadjuvant study evaluating the effect of a novel combination regimen of Pertuzumab and Trastuzumab plus chemotherapy (Docetaxel) in women with early-stage HER2-positive breast cancer, presented at the CTRC-AACR San Antonio Breast Cancer Symposium (SABCS), Dec. 8-12, 2010, showed that the two HER2 antibodies plus Docetaxel given in the neoadjuvant setting prior to surgery significantly improved the rate of complete tumor disappearance (pathological complete response rate, pCR, of 45.8 percent) in the breast by more than half compared to Trastuzumab plus Docetaxel (pCR of 29.0 percent), p=0.014.

Pertuzumab, marketed under the tradename PERJETA®, was approved in 2012 for the treatment of patients with advanced or late-stage (metastatic) HER2-positive breast cancer. HER2-positive breast cancers have increased amounts of the HER2 protein that contributes to cancer cell growth and survival.

On Sep. 30, 2013, the U.S. Food and Drug Administration granted accelerated approval to PERJETA® (pertuzumab) as part of a complete treatment regimen for patients with early stage breast cancer (EBC) before surgery (neoadjuvant setting). PERJETA® is the first FDA-approved drug for the neoadjuvant treatment of breast cancer.

Patent Publications related to HER2 antibodies include: U.S. Pat. Nos. 5,677,171; 5,720,937; 5,720,954; 5,725,856; 5,770,195; 5,772,997; 6,165,464; 6,387,371; 6,399,063; 6,015,567; 6,333,169; 4,968,603; 5,821,337; 6,054,297; 6,407,213; 6,639,055; 6,719,971; 6,800,738; 8,075,890; 5,648,237; 7,018,809; 6,267,958; 6,685,940; 6,821,515; 7,060,268; 7,682,609; 7,371,376; 6,127,526; 6,333,398; 6,797,814; 6,339,142; 6,417,335; 6,489,447; 7,074,404; 7,531,645; 7,846,441; 7,892,549; 8,075,892; 6,573,043; 6,905,830; 7,129,051; 7,344,840; 7,468,252; 7,674,589; 7,919,254; 6,949,245; 7,485,302; 7,498,030; 7,501,122; 7,537,931; 7,618,631; 7,862,817; 7,041,292; 6,627,196; 7,371,379; 6,632,979; 7,097,840; 7,575,748; 6,984,494; 7,279,287; 7,811,773; 7,993,834; 8,076,066; 8,044,017; 7,435,797; 7,850,966; 7,485,704; 7,807,799; 8,142,784; 7,560,111; 7,879,325; 8,241,630; 7,449,184; 8,163,287; 7,700,299; 7,981,418; 8,247,397; and US 2010/0016556; US 2005/0244929; US 2001/0014326; US 2003/0202972; US 2006/0099201; US 2010/0158899; US 2011/0236383; US 2011/0033460; US 2008/0286280; US 2005/0063972; US 2006/0182739; US 2009/0220492; US 2003/0147884; US 2004/0037823; US 2005/0002928; US 2007/0292419; US 2008/0187533; US 2011/0250194; US 2012/0034213; US 2003/0152987; US 2005/0100944; US 2006/0183150; US 2008/0050748; US 2009/0155803; US 2010/0120053; US 2005/0244417; US 2007/0026001; US 2008/0160026; US 2008/0241146; US 2005/0208043; US 2005/0238640; US 2006/0034842; US 2006/0073143; US 2006/0193854; US 2006/0198843; US 2011/0129464; US 2007/0184055; US 2007/0269429; US 2008/0050373; US 2006/0083739; US 2009/0087432; US 2006/0210561; US 2002/0035736; US 2002/0001587; US 2008/0226659; US 2002/0090662; US 2006/0046270; US 2008/0108096; US 2007/0166753; US 2008/0112958; US 2009/0239236; US 2012/0034609; US 2012/0093838; US 2004/0082047; US 2012/0065381; US 2009/0187007; US 2011/0159014; US 2004/0106161; US 2011/0117096; US 2004/0258685; US 2009/0148402; US 2009/0099344; US 2006/0034840; US 2011/0064737; US 2005/0276812; US 2008/0171040; US 2009/0202536; US 2006/0013819; US 2012/0107391; US 2006/0018899; US 2009/0285837; US 2011/0117097; US 2006/0088523; US 2010/0015157; US 2006/0121044; US 2008/0317753; US 2006/0165702; US 2009/0081223; US 2006/0188509; US 2009/0155259; US 2011/0165157; US 2006/0204505; US 2006/0212956; US 2006/0275305; US 2012/0003217; US 2007/0009976; US 2007/0020261; US 2007/0037228; US 2010/0112603; US 2006/0067930; US 2007/0224203; US 2011/0064736; US 2008/0038271; US 2008/0050385; US 2010/0285010; US 2011/0223159; US 2008/0102069; US 2010/0008975; US 2011/0245103; US 2011/0246399; US 2011/0027190; US 2010/0298156; US 2011/0151454; US 2011/0223619; US 2012/0107302; US 2009/0098135; US 2009/0148435; US 2009/0202546; US 2009/0226455; US 2009/0317387; US 2011/0044977; US 2012/0121586.

Cancer Immunotherapy by Targeting PD-L1

Cancer immunotherapy aims to work with a patient's own immune system to enable the body to recognize and kill tumor cells. Tumors can evade the immune system through various mechanisms, such as overexpression of programmed death-ligand 1 (PD-L1). This has been observed throughout the tumor microenvironment, as seen in clinical trials across multiple tumor types, making PD-L1 a target for cancer immunotherapy. Binding of PD-L1 to either of its receptors, B7.1 or PD-1, on the surface of T cells results in deactivation of the T cells. This deactivation occurs when T cells bind to either tumor cells or tumor-infiltrating immune cells, such as T regulatory cells and macrophages. (See, e.g., Chen D S, et al., (2012) Clin Cancer Res. 18:6580-6587, and Murphy K, Janeway's Immunobiology. 8th ed. New York, N.Y.: Garland Science; 2012).

Atezolizumab (also referred to as MPDL3280A) is a humanized monoclonal antibody of IgG1 isotype that is designed to prevent PD-L1 from binding to B7.1 and PD-1 (CAS Registry Number 1380723-44-3). Sequences for the antibody are provided in WO 2010/077634. The inhibition of PD-L1 may prevent the deactivation of T cells. T cells may then detect tumor cells and release cytotoxic granzymes to trigger tumor cell death. This process may further stimulate the immune response by recruiting more T cells to target the tumor, thus propagating the immune response (see, e.g., Chen D S, et al., (2012) Clin Cancer Res. 18:6580-6587; Murphy K., Janeway's Immunobiology. 8th ed. New York, N.Y.: Garland Science; 2012; Keir M E, et al., (2008). Annu Rev Immunol. 26:677-704; and Chen D S, and Mellman I. (2013) Immunity 39:1-10).

Atezolizumab is thought to preserve the interaction between PD-L2 and PD-1. PD-L2 is another ligand that helps maintain immune homeostasis. It is infrequently expressed on tumor cells, but it can be highly expressed in normal tissues. Because atezolizumab is designed to bind to PD-L1, it is not believed to interfere with PD-L2 interactions. Therefore, PD-L2 may remain free to bind to PD-1 (see, e.g., Chen D S, et al., (2012) Clin Cancer Res. 18:6580-6587; and Topalian S L, et al., (2012) Curr Opin Immunol., 24:207-212).

Atezolizumab is also engineered to eliminate antibody-dependent cell-mediated cytotoxicity (ADCC). ADCC is a mechanism by which the immune system targets antibody-bound cells for destruction. The atezolizumab (MPDL3280A) antibody is engineered to remove the structural component responsible for ADCC. As a result, atezolizumab (MPDL3280A) is not believed to deplete other immune cells expressing PD-L (see, e.g., Sharon E, et al. (2014) Chin J Cancer. 33:434-444.

### SUMMARY OF THE INVENTION

The invention relates to methods of using a PD-1 binding antagonist or a PD-L1 binding antagonist, in combination with a HER2-targeted therapy, for the treatment of HER2 positive breast cancer. In certain embodiments, the PD-L1 binding antagonist is an anti-PD-L1 antibody, e.g., atezolizumab (MPDL3280A); and the HER2-targeted therapy is trastuzumab, pertuzumab, trastuzumab emtansine, or combinations of the foregoing. In particular, the HER2-targeted therapy is a combination of trastuzumab and pertuzumab; or trastuzumab emtansine. The methods may comprise treatment in the neoadjuvant, adjuvant or metastatic setting. In certain embodiments, the methods comprise treatment in the neoadjuvant setting or in the metastatic setting, including first line or subsequent metastatic settings. In certain embodiments, e.g., in the neoadjuvant setting, additional chemotherapy and other treatments may be admininstered prior to definitive surgery.

In various embodiments, the HER2 positive breast cancer is first line metastatic HER2 positive breast cancer, or operable or locally advanced HER2 positive breast cancer, or HER2 positive inflammatory early breast cancer.

In one aspect, a method of treating HER2 positive breast cancer is provided, the method comprising administering to a patient having said breast cancer a therapeutically effective amount of an anti-PD-L1 antibody in combination with trastuzumab and pertuzumab. In certain embodiments, the anti-PD-L1 antibody comprises (a) an HVR-H1 sequence of GFTFSDSWIH (SEQ ID NO:8); (b) an HVR-H2 sequence of AWISPYGGSTYYADSVKG (SEQ ID NO:9); (c) an HVR-H3 sequence of RHWPGGFDY (SEQ ID NO:10); (d) an HVR-L1 sequence of RASQDVSTAVA (SEQ ID NO:15); (e) an HVR-L2 sequence of SASFLYS, (SEQ ID NO:16); and (f) an HVR-L3 sequence of QQYLYHPAT (SEQ ID NO:17). In certain embodiments, the anti-PD-L1 antibody comprises the heavy chain variable region of SEQ ID NO:3 and the light chain variable region of SEQ ID NO:4. In certain embodiments, the anti-PD-L1 antibody is atezolizumab. In certain embodiments, atezolizumab is administered by infusion at a dose of 1200 mg on the first day of treatment and every three weeks thereafter; trastuzumab is administered by infusion at a loading dose of 8 mg/kg on the first day of treatment and at a dose of 6 mg/kg every three weeks thereafter; and pertuzumab is administered by infusion at a loading dose of 840 mg on the first day of treatment and at a dose of 420 mg every three weeks thereafter. In any of the above embodiments, the HER2 positive breast cancer is first line metastatic HER2 positive breast cancer.

Alternatively, in any of the above embodiments, the treatment is given as neoadjuvant therapy. In certain embodiments, the method comprises administering atezolizumab in combination with trastuzumab and pertuzumab, and wherein atezolizumab is administered by infusion at a dose of 1200 mg on the first day of treatment and every three weeks thereafter; trastuzumab is administered by infusion at a loading dose of 8 mg/kg on the first day of treatment and at a dose of 6 mg/kg every three weeks thereafter; and pertuzumab is administered by infusion at a loading dose of 840 mg on the first day of treatment and at a dose of 420 mg every three weeks thereafter. In certain embodiments, atezolizumab is administered in combination with trastuzumab and pertuzumab every three weeks for two cycles, followed by administration of a therapeutic regimen comprising chemotherapy. In certain embodiments, the therapeutic regimen comprising chemotherapy comprises trastuzumab, pertuzumab, carboplatin and docetaxel. In certain embodiments, carboplatin is administered by infusion at a dose of 6 mg/ml·min every three weeks; docetaxel is administered by infusion at a dose of 75 mg/m² every three weeks; trastuzumab is administered by infusion at a dose of 6 mg/kg every three weeks; and pertuzumab is administered by infusion at a dose of 420 mg every three weeks. In certain of the preceding embodiments, the therapeutic regimen comprising chemotherapy is administered for six cycles. In certain embodiments, after the six cycles of the therapeutic regimen comprising chemotherapy, the patient is subjected to definitive surgery. In certain embodiments, after definitive surgery, trastuzumab is administered to the patient. In certain embodiments, after definitive surgery, trastuzumab is administered to the patient by infusion at a dose of 6 mg/kg every three weeks. In certain embodiments, after definitive surgery, trastuzumab is administered to the patient by infusion at a dose of 6 mg/kg every three weeks for twelve cycles.

In a further aspect, a method of treating HER2 positive breast cancer is provided, the method comprising administering to a patient having said breast cancer a therapeutically effective amount of an anti-PD-L1 antibody in combination with trastuzumab emtansine. In certain embodiments, the anti-PD-L1 antibody comprises (a) an HVR-H1 sequence of GFTFSDSWIH (SEQ ID NO:8); (b) an HVR-H2 sequence of AWISPYGGSTYYADSVKG (SEQ ID NO:9); (c) an HVR-H3 sequence of RHWPGGFDY (SEQ ID NO:10); (d) an HVR-L1 sequence of RASQDVSTAVA (SEQ ID NO:15); (e) an HVR-L2 sequence of SASFLYS, (SEQ ID NO:16); and (f) an HVR-L3 sequence of QQYLYHPAT (SEQ ID NO:17). In certain embodiments, the anti-PD-L1 antibody comprises the heavy chain variable region of SEQ ID NO:3 and the light chain variable region of SEQ ID NO:4. In certain embodiments, the anti-PD-L1 antibody is atezolizumab. In certain embodiments, atezolizumab is administered by infusion at a dose of 1200 mg every three weeks and trastuzumab emtansine is administered by infusion at dose of 3.6 mg/kg every three weeks. In any of the above embodiments, the HER2 positive breast cancer is first line metastatic HER2 positive breast cancer. Alternatively, in any of the above embodiments, the HER2 positive breast cancer is metastatic breast cancer and the patient has received prior treatment with trastuzumab and a taxane. In either of the preceding embodiments, the anti-PD-L1 antibody is atezolizumab, and wherein the atezolizumab is administered by infusion at a dose of 1200 mg every three weeks and trastuzumab emtansine is administered by infusion at dose of 3.6 mg/kg every three weeks.

Alternatively, in any of the above embodiments, the treatment is given as neoadjuvant therapy. In certain embodiments, the method comprises administering atezolizumab in combination with trastuzumab emtansine, and wherein atezolizumab is administered by infusion at a dose of 1200 mg every three weeks and trastuzumab emtansine is administered by infusion at dose of 3.6 mg/kg every three weeks. In certain embodiments, atezolizumab in combination with trastuzumab emtansine is administered every three weeks for two cycles, followed by administration of a therapeutic regimen comprising chemotherapy. In certain embodiments, the therapeutic regimen comprising chemotherapy comprises carboplatin, docetaxel, trastuzumab and pertuzumab. In certain embodiments, carboplatin is administered by infusion at a dose of 6 mg/ml·min every three weeks; docetaxel is administered by infusion at a dose of 75 mg/m² every three weeks; trastuzumab is administered by infusion at a loading dose of 8 mg/kg on the first day of treatment with trastuzumab, and at a dose of 6 mg/kg every three weeks thereafter; and pertuzumab is administered by infusion at a loading dose of 840 mg on the first day of treatment with pertuzumab, and at a dose of 420 mg every three weeks thereafter. In certain of the preceding embodiments, the therapeutic regimen comprising chemotherapy is administered for six cycles. In certain embodiments, after the six cycles of the therapeutic regimen comprising chemotherapy, the patient is subjected to definitive surgery. In certain embodiments, after definitive surgery, trastuzumab is administered to the patient. In certain embodiments, after definitive surgery, trastuzumab is administered to the patient by infusion at a dose of 6 mg/kg every three weeks. In certain embodiments, after definitive surgery, trastuzumab is administered to the patient by infusion at a dose of 6 mg/kg every three weeks for twelve cycles

### BRIEF DESCRIPTION OF THE DRAWINGS

FIG. 1 depicts Stage 1 of the Phase Ib clinical study described in the Examples. The study schema for the safety evaluation cohorts (Cohorts 1A and 1B) is shown.

FIG. 2 depicts one aspect of Stage 2 of the study. The atezolizumab/trastuzumab emtansine safety expansion cohort (Cohort 2C) is shown.

FIG. 3 depicts a further aspect of Stage 2 of the study. The “neoadjuvant window cohorts” (Cohorts 2A and 2B) are shown. This schema represents baseline scenario that both cohorts are tested in neoadjuvant portion of the study. No randomization will be employed if only one cohort moves forward (n=20 patients). ^{a}Loading dose of trastuzumab 8 mg/kg and pertuzumab 840 mg administered with firs cycle of THCP (docetaxel, carboplatin, trastuzumab and pertuzumab) patients who did not receive trastuzumab or pertuzumab during the two cycles of Neoadjuvant Window therapy with trastuzumab. EBC=early breast cancer, HER2=human epidermal growth factor 2; LABC=metaststic breast cancer; LVEF=left ventricular ejection fraction; MBC=metastatic breast cancer.

FIG. 4 depicts the schema of HER2-directed therapy management based on left ventricular ejection fraction (LVEF). EF=ejection fraction.

FIG. 5 depicts the schema of the Phase II clinical study described in Example 4. ECOG=Eastern Cooperative Oncology Group; HER2=human epidermal growth factor receptor; IV=intravenous; LABC=locally advanced breast cancer; MBC=metaststic breast cancer; PD-L1=programmed death ligand-1; q3w=every 3 weeks; RECIST=response evaluation criteriain solid tumors; T-DM1-trastuzumab emtansine; tx=treatment. Stratification factors: Word region (W. Europe vs. U.S. vs. Rest of World). Tumor PD-L1 Status (IC 0 vs. IC 1/2/3); Liver metasteses (yes vs. no).

FIG. 6 depicts the overview of tissue flow in the Phase II clinical study (see Example 7).

FIG. 7 Algorithm for Continuation and Discontinuation of Trastuzumab Emtansine Treatment Based on LVEF Assessments in Patients. LVEF assessment results must be reviewed before the next scheduled trastuzumab emtansine infusion. ^{a} LVEF<40% should be repeated within 21 days, and trastuzumab emtansine treatment should be discontinued if LVEF<40% is confirmed. Trastuzumab emtansine should be held while the confirmatory LVEF measurement is obtained. ^{b} After a second consecutive confirmatory measurement is obtained, trastuzumab emtansine treatment should be discontinued if the ≥10% absolute LVEF decrease from baseline is confirmed and if medical management was required in order to correct the LVEF. CHF=congestive heart failure; LVEF=left ventricular ejection fraction; T-DM1=trastuzumab emtansine.

FIGS. 8A and 8B show the amino acid sequences of Trastuzumab light chain (FIG. 8A; SEQ ID NO. 1) and heavy chain (FIG. 4B; SEQ ID NO. 2), respectively. Boundaries of the variable light and variable heavy domains are indicated by arrows.

FIGS. 9A and 9B show the amino acid sequence of Pertuzumab light chain (FIG. 9A; SEQ ID NO. 18) and heavy chain (FIG. 9B; SEQ ID No. 19). CDRs are shown in bold.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described.

All references cited throughout the disclosure are expressly incorporated by reference herein in their entirety. In the event that one or more of the incorporated literature, patents, and similar materials differs from or contradicts this application, including but not limited to defined terms, term usage, described techniques, or the like, this application controls.

### Definitions

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Reference to a tumor or cancer as a “Stage 0,” “Stage I,” “Stage II,” “Stage III,” or “Stage IV”, and various sub-stages within this classification, indicates classification of the tumor or cancer using the Overall Stage Grouping or Roman Numeral Staging methods known in the art. Although the actual stage of the cancer is dependent on the type of cancer, in general, a Stage 0 cancer is an in situ lesion, a Stage I cancer is small localized tumor, a Stage II and III cancer is a local advanced tumor which exhibits involvement of the local lymph nodes, and a Stage IV cancer represents metastatic cancer. The specific stages for each type of tumor is known to the skilled clinician.

The term “metastatic breast cancer” means the state of breast cancer where the cancer cells are transmitted from the original site to one or more sites elsewhere in the body, by the blood vessels or lymphatics, to form one or more secondary tumors in one or more organs besides the breast.

The term “first line” metastatic breast cancer or “previously untreated” or “treatment naïve” metastatic breast cancer refers to metastatic breast cancer that has not received treatment in the metastatic setting.

As used herein, the term “locally advanced” breast cancer refers to progressive or recurrent locally advanced breast cancer.

The term “prior treatment,” with reference to a taxane, refers to treatment that has occurred prior to the first line metastatic or locally advanced setting. For example, “prior treatment” may refer to treatment in the neoadjuvant, adjuvant or other setting prior to the first line metastatic or locally advanced setting.

An “advanced” cancer is one which has spread outside the site or organ of origin, either by local invasion or metastasis. Accordingly, the term “advanced” cancer includes both locally advanced and metastatic disease.

A “refractory” cancer is one which progresses even though an anti-tumor agent, such as a chemotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

An“operable” or “resectable” cancer is cancer which is confined to the primary organ and suitable for surgery (resection).

A “non-resectable” or “unresectable” cancer is not able to be removed (resected) by surgery.

A “HER2-positive” cancer comprises cancer cells which have higher than normal levels of HER2. Examples of HER2-positive cancer include HER2-positive breast cancer and HER2-positive gastric cancer. Optionally, HER2-positive cancer has an immunohistochemistry (IHC) score of 2+ or 3+ and/or an in situ hybridization (ISH) amplification ratio ≥2.0.

Herein, a “patient” or “subject” is a human patient. The patient may be a “cancer patient,” i.e. one who is suffering or at risk for suffering from one or more symptoms of cancer, in particular gastric or breast cancer.

A “patient population” refers to a group of cancer patients. Such populations can be used to demonstrate statistically significant efficacy and/or safety of a drug, such as Pertuzumab.

A “relapsed” patient is one who has signs or symptoms of cancer after remission. Optionally, the patient has relapsed after adjuvant or neoadjuvant therapy.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

“Neoadjuvant therapy” or “preoperative therapy” herein refers to therapy given prior to surgery. The goal of neoadjuvant therapy is to provide immediate systemic treatment, potentially eradicating micrometastases that would otherwise proliferate if the standard sequence of surgery followed by systemic therapy were followed. Neoadjuvant therapy may also help to reduce tumor size thereby allowing complete resection of initially unresectable tumors or preserving portions of the organ and its functions. Furthermore, neoadjuvant therapy permits an in vivo assessment of drug efficacy, which may guide the choice of subsequent treatments.

“Adjuvant therapy” herein refers to therapy given after definitive surgery, where no evidence of residual disease can be detected, so as to reduce the risk of disease recurrence. The goal of adjuvant therapy is to prevent recurrence of the cancer, and therefore to reduce the chance of cancer-related death. Adjuvant therapy herein specifically excludes neoadjuvant therapy.

“Survival” refers to the patient remaining alive, and includes disease free survival (DFS), progression free survival (PFS) and overall survival (OS). Survival can be estimated by the Kaplan-Meier method, and any differences in survival are computed using the stratified log-rank test.

“Progression-Free Survival” (PFS) is the time from the first day of treatment to documented disease progression (including isolated CNS progression) or death from any cause on study, whichever occurs first.

“Disease free survival (DFS)” refers to the patient remaining alive, without return of the cancer, for a defined period of time such as about 1 year, about 2 years, about 3 years, about 4 years, about 5 years, about 10 years, etc., from initiation of treatment or from initial diagnosis. In one aspect of the invention, DFS is analyzed according to the intent-to-treat principle, i.e., patients are evaluated on the basis of their assigned therapy. The events used in the analysis of DFS can include local, regional and distant recurrence of cancer, occurrence of secondary cancer, and death from any cause in patients without a prior event (e.g, breast cancer recurrence or second primary cancer).

“Overall survival” refers to the patient remaining alive for a defined period of time, such as about 1 year, about 2 years, about 3 years, about 4 years, about 5 years, about 10 years, etc., from initiation of treatment or from initial diagnosis. In the studies underlying the invention the event used for survival analysis was death from any cause.

By “extending survival” is meant increasing DFS and/or OS in a treated patient relative to an untreated patient, or relative to a control treatment protocol. Survival is monitored for at least about six months, or at least about 1 year, or at least about 2 years, or at least about 3 years, or at least about 4 years, or at least about 5 years, or at least about 10 years, etc., following the initiation of treatment or following the initial diagnosis.

“Hazard ratio” in survival analysis is a summary of the difference between two survival curves, representing the reduction in the risk of death on treatment compared to control, over a period of follow-up. Hazard ratio is a statistical definition for rates of events. For the purpose of the invention, hazard ratio is defined as representing the probability of an event in the experimental arm divided by the probability of an event in the control arm at any specific point in time.

By “monotherapy” is meant a therapeutic regimen that includes only a single therapeutic agent for the treatment of the cancer or tumor during the course of the treatment period.

By “maintenance therapy” is meant a therapeutic regimen that is given to reduce the likelihood of disease recurrence or progression. Maintenance therapy can be provided for any length of time, including extended time periods up to the life-span of the subject. Maintenance therapy can be provided after initial therapy or in conjunction with initial or additional therapies. Dosages used for maintenance therapy can vary and can include diminished dosages as compared to dosages used for other types of therapy.

“Definitive surgery” is used as that term is used within the medical community. Definitive surgery includes, for example, procedures, surgical or otherwise, that result in removal or resection of the tumor, including those that result in the removal or resection of all grossly visible tumor. Definitive surgery includes, for example, complete or curative resection or complete gross resection of the tumor. Definitive surgery includes procedures that occurs in one or more stages, and includes, for example, multi-stage surgical procedures where one or more surgical or other procedures are performed prior to resection of the tumor. Definitive surgery includes procedures to remove or resect the tumor including involved organs, parts of organs and tissues, as well as surrounding organs, such as lymph nodes, parts of organs, or tissues.

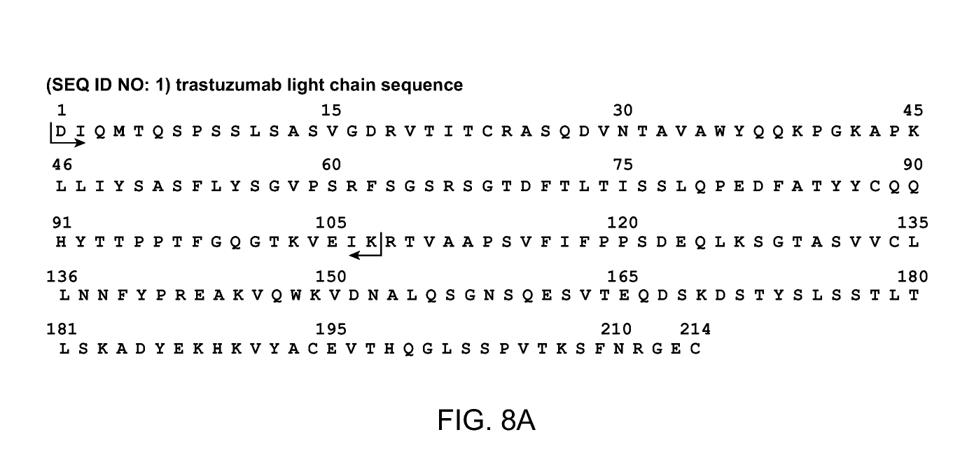

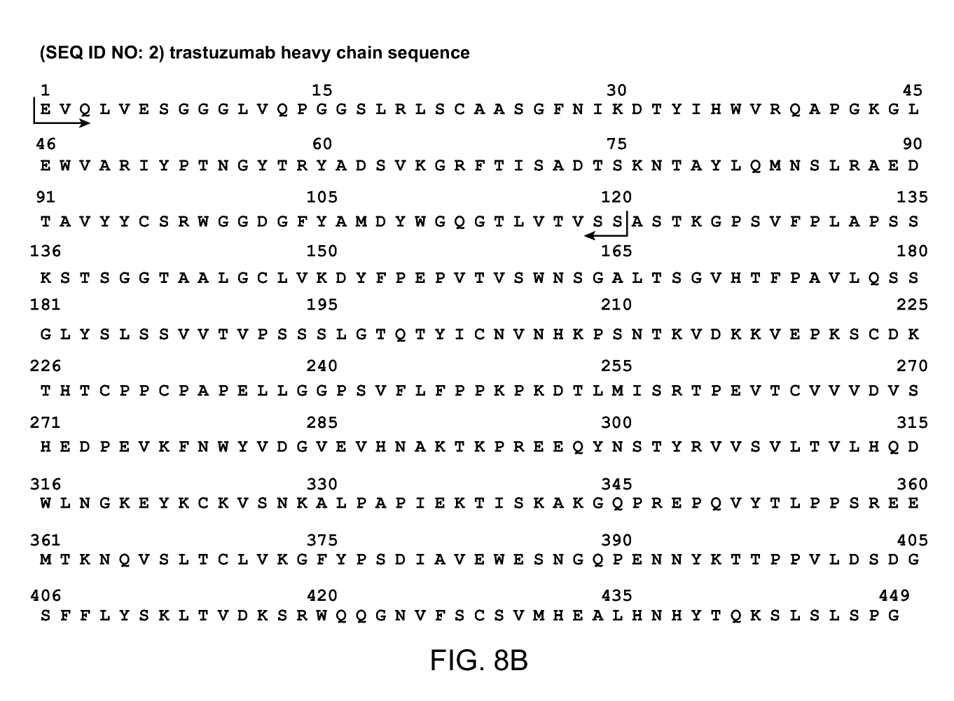

As defined herein, the terms “trastuzumab”, “HERCEPTIN®” and “huMAb4D5-8” are used interchangeably. Such antibody preferably comprises the light and heavy chain amino acid sequences shown in FIG. 8A (SEQ ID NO: 1) and FIG. 8B (SEQ ID NO. 2), respectively.

The “epitope 4D5” or “4D5 epitope” or “4D5” is the region in the extracellular domain of HER2 to which the antibody 4D5 (ATCC CRL 10463) and trastuzumab bind. This epitope is close to the transmembrane domain of HER2, and within Domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 529 to about residue 625, inclusive, of HER2).

The “epitope 2C4” or “2C4 epitope” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of HER2. Epitope 2C4 comprises residues from domain II in the extracellular domain of HER2. The 2C4 antibody and Pertuzumab bind to the extracellular domain of HER2 at the junction of domains I, II and III (Franklin et al. Cancer Cell 5:317-328 (2004)).

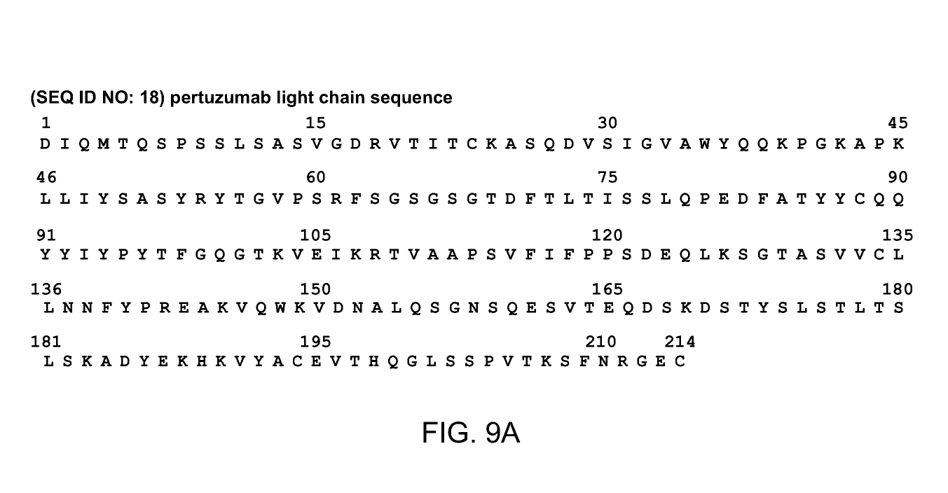

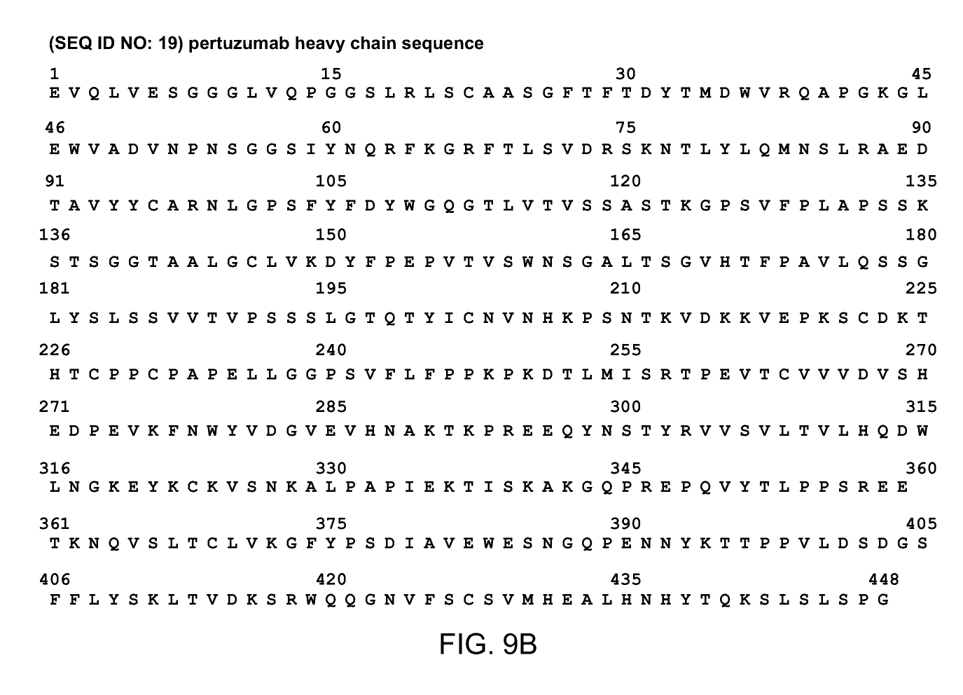

For the purposes herein, “pertuzumab”, “PERJETA®” and “rhuMAb 2C4”, are used interchangeably. Such antibody preferably comprises the light and heavy chain amino acid sequences in SEQ ID NOs: 18 and 19, respectively (FIGS. 9A and 9B, respectively). The antibody is optionally produced by recombinant Chinese Hamster Ovary (CHO) cells.

As defined herein, the terms “T-DM1,” “trastuzumab-MCC-DM1,” “ado-trastuzumab emtansine,” “trastuzumab emtansine,” and “KADCYLA®” are used interchangeably, and refer to trastuzumab linked through the linker moiety MCC to the maytansinoid drug moiety DM1, including all mixtures of variously loaded and attached antibody-drug conjugates where 1, 2, 3, 4, 5, 6, 7, and 8 drug moieties are covalently attached to the antibody trastuzumab (U.S. Pat. No. 7,097,840; US 2005/0276812; US 2005/0166993).

Herein, an “anti-tumor agent” refers to a drug used to treat cancer. Non-limiting examples of anti-tumor agents herein include chemotherapy agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

A “chemotherapy” is use of a chemotherapeutic agent useful in the treatment of cancer.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI). The term “effective amount” specifically includes an amount suitable for achieving any of the primary or secondary endpoints of the clinical trial described in Example 1.

A “taxane” is a chemotherapy which inhibits mitosis and interferes with microtubules. Examples of taxanes include paclitaxel (TAXOL®; Bristol-Myers Squibb Oncology, Princeton, N.J.); cremophor-free, albumin-engineered nanoparticle formulation of paclitaxel or nab-paclitaxel (ABRAXANE™; American Pharmaceutical Partners, Schaumberg, Ill.); and docetaxel (TAXOTERE®; Rhône-Poulenc Rorer, Antony, France).

An “anthacycline” is a type of antibiotic that comes from the fungus Streptococcus peucetius, examples include: daunorubicin, doxorubicin, and epirubicin, etc.

“Anthracycline-based chemotherapy” refers to a chemotherapy regimen that consists of or include one or more anthracycline. Examples include 5-FU, epirubicin, and cyclophosphamide (FEC); 5-FU, doxorubicin, and cyclophosphamide (FAC); doxorubicin and cyclophosphamide (AC); epirubicin and cyclophosphamide (EC); etc.

For the purposes herein, “carboplatin-based chemotherapy” refers to a chemotherapy regimen that consists of or includes one or more carboplatins. An example is TCH (docetaxel/TAXOL®, carboplatin, and trastuzumab/HERCEPTIN®).

An “aromatase inhibitor” inhibits the enzyme aromatase, which regulates estrogen production in the adrenal glands. Examples of aromatase inhibitors include: 4(5)-imidazoles, aminoglutethimide, MEGASE® megestrol acetate, AROMASIN® exemestane, formestane, fadrozole, RIVISOR® vorozole, FEMARA® letrozole, and ARIMIDEX® anastrozole. In one embodiment, the aromatase inhibitor herein is letrozole or anastrozole.

An “antimetabolite chemotherapy” is use of an agent which is structurally similar to a metabolite, but can not be used by the body in a productive manner. Many antimetabolite chemotherapeutic agents interfere with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

The term “platin” is used herein to refer to platinum based chemotherapy, including, without limitation, cisplatin, carboplatin, and oxaliplatin.

The term “fluoropyrimidine” is used herein to refer to an antimetabolite chemotherapy, including, without limitation, capecitabine, floxuridine, and fluorouracil (5-FU).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinal chloride.

By “co-administering” is meant intravenously administering two (or more) drugs during the same administration, rather than sequential infusions of the two or more drugs. Generally, this will involve combining the two (or more) drugs into the same IV bag prior to co-administration thereof.

A drug that is administered “concurrently” with one or more other drugs is administered during the same treatment cycle, on the same day of treatment as the one or more other drugs, and, optionally, at the same time as the one or more other drugs. For instance, for cancer therapies given every 3 weeks, the concurrently administered drugs are each administered on day-1 of a 3-week cycle.

“Cardiac toxicity” refers to any toxic side effect that affects the heart and that results from administration of a drug or drug combination. Cardiac toxicity can be evaluated based on any one or more of: incidence of symptomatic left ventricular systolic dysfunction (LVSD) or congestive heart failure (CHF), or decrease in left ventricular ejection fraction (LVEF).

The phrase “without increasing cardiac toxicity” for a drug combination including pertuzumab refers to an incidence of cardiac toxicity that is equal or less than that observed in patients treated with drugs other than pertuzumab in the drug combination (e.g. equal or less than that resulting from administration of trastuzumab and the chemotherapy, e.g. docetaxel).

A “vial” is a container suitable for holding a liquid or lyophilized preparation. In one embodiment, the vial is a single-use vial, e.g. a 20-cc single-use vial with a stopper.

The term “package insert” is used to refer to instructions customarily included in commercial packages of therapeutic products, that contain information about the indications, usage, dosage, administration, contraindications and/or warnings concerning the use of such therapeutic products.

An “adverse event” is any unfavorable and unintended sign, symptom, or disease temporally associated with the use of an investigational (medicinal) product or other protocol-imposed intervention, regardless of attribution; and includes: AEs not previously observed in the patient that emerge during the protocol-specified AE reporting period, including signs or symptoms associated with breast cancer that were not present before the AE reporting period; complications that occur as a result of protocol-mandated interventions (e.g., invasive procedures such as biopsies); if applicable, AEs that occur before assignment of study treatment associated with medication washout, no treatment run-in, or other protocol-mandated intervention; Preexisting medical conditions (other than the condition being studied) judged by the investigator to have worsened in severity or frequency or changed in character during the protocol-specified AE reporting period

An adverse event is classified as a “Serious Adverse Events” (SAE) if it meets the following criteria: results in death (i.e., the AE actually causes or leads to death); life threatening (i.e., the AE, in the view of the investigator, places the patient at immediate risk of death, but not including an AE that, had it occurred in a more severe form, might have caused death); requires or prolongs inpatient hospitalization; results in persistent or significant disability/incapacity (i.e., the AE results in substantial disruption of the patient's ability to conduct normal life functions); results in a congenital anomaly/birth defect in a neonate/infant born to a mother exposed to the investigational product; or is considered a significant medical event by the investigator based on medical judgment (e.g., may jeopardize the patient or may require medical/surgical intervention to prevent one of the outcomes listed above). All AEs that do not meet any of the criteria for serious are regarded as non-serious AEs. The terms “severe” and “serious” are not synonymous. Severity (or intensity) refers to the grade of a specific AE, e.g., mild (Grade 1), moderate (Grade 2), or severe (Grade 3) myocardial infarction. “Serious” is a regulatory definition (see previous definition) and is based on patient or event outcome or action criteria usually associated with events that pose a threat to a patient's life or functioning. Seriousness (not severity) serves as the guide for defining regulatory reporting obligations from the Sponsor to applicable regulatory authorities. Severity and seriousness should be independently assessed when recording AEs and SAEs on the eCRF.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (pembrolizumab) described herein. In another specific aspect, a PD-1 binding antagonist is CT-011 (pidilizumab) described herein. In another specific aspect, a PD-1 binding antagonist is MEDI-0680 (AMP-514) described herein. In another specific aspect, a PD-1 binding antagonist is PDR001 described herein. In another specific aspect, a PD-1 binding antagonist is REGN2810 described herein. In another specific aspect, a PD-1 binding antagonist is BGB-108 described herein.

The term “PD-L1 binding antagonist” refers to a molecule that decreases, blocks, inhibits, abrogates or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is MPDL3280A (atezolizumab) described herein. In another specific aspect, an anti-PD-L1 antibody is MDX-1105 described herein. In still another specific aspect, an anti-PD-L1 antibody is YW243.55.S70 described herein. In still another specific aspect, an anti-PD-L1 antibody is MEDI4736 (durvalumab) described herein. In still another specific aspect, an anti-PD-L1 antibody is MSB0010718C (avelumab) described herein.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

### DETAILED DESCRIPTION

The invention relates to methods of using a PD-1 binding antagonist or a PD-L1 binding antagonist, in combination with a HER2-targeted therapy, for the treatment of HER2 positive breast cancer. In certain embodiments, the PD-L1 binding antagonist is an anti-PD-L1 antibody, e.g., atezolizumab (MPDL3280A); and the HER2-targeted therapy is trastuzumab, pertuzumab, trastuzumab emtansine, or combinations of the foregoing. In particular, the HER2-targeted therapy is a combination of trastuzumab and pertuzumab; or trastuzumab emtansine. The methods may comprise treatment in the neoadjuvant, adjuvant or metastatic setting. In certain embodiments, the methods comprise treatment in the neoadjuvant setting or in the metastatic setting, including first line or subsequent metastatic settings. In certain embodiments, e.g., in the neoadjuvant setting, additional chemotherapy and other treatments may be administered prior to definitive surgery

Anti-PD-L1 Antibodies

In certain embodiments, an anti-PD-L1 antibody is used in the treatment methods provided herein. PD-L1 (programmed death ligand 1), also known as PDL1, B7-H1, B7-4, CD274, and B7-H, is a transmembrane protein, and its interaction with PD-1 inhibits T-cell activation and cytokine production. In some embodiments, the anti-PD-L1 antibody described herein binds to human PD-L1. Examples of anti-PD-L1 antibodies that can be used in the methods described herein are described in PCT patent application WO 2010/077634 A1 and U.S. Pat. No. 8,217,149, which are incorporated herein by reference in their entirety. Further examples of anti-PD-L1 antibodies useful that can be used in the methods described herein are described in PCT patent application WO 2007/005874, WO 2011/066389, and US 2013/034559, which are incorporated herein by reference in their entirety. The anti-PD-L1 antibodies useful in this invention, including compositions containing such antibodies, may be used in combination with Her2-targeted therapies to treat HER2 positive breast cancer.

In some embodiments, an anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)₂ fragments. In some embodiments, the anti-PD-L1 antibody is a humanized antibody. In some embodiments, the anti-PD-L1 antibody is a human antibody.

Anti-PD-L1 antibodies described in WO 2010/077634 A1 and U.S. Pat. No. 8,217,149 may be used in the methods described herein. In some embodiments, the anti-PD-L1 antibody is atezolizumab (MPDL3280, CAS Registry Number 1380723-44-3). In certain embodiments, the anti-PD-L1 antibody comprises a heavy chain variable region sequence of SEQ ID NO:3 and a light chain variable region sequence of SEQ ID NO:4. In a still further embodiment, provided is an isolated anti-PD-L1 antibody comprising a heavy chain variable region and/or a light chain variable region sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: EVQLVESGGGLVQPGGSLRLSCAASGFTFSDSWIHWVRQAPGKGLEWVAWISPY GGSTYYADSVKGRFTISADTSKNTAYLQMNSLRAEDTAVYYCARRHWPGGFDY WGQGTLVTVSS (SEQ ID NO:3), and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In one embodiment, the anti-PD-L1 antibody comprises a heavy chain variable region comprising an HVR-H1, HVR-H2 and HVR-H3 sequence, wherein:

(a) the HVR-H1 sequence is GFTFSX₁SWIH (SEQ ID NO:5);

(b) the HVR-H2 sequence is AWIX₂PYGGSX₃YYADSVKG (SEQ ID NO:6); and

(c) the HVR-H3 sequence is RHWPGGFDY (SEQ ID NO:7);

further wherein: X₁ is D or G; X₂ is S or L; X₃ is T or S.
In one specific aspect, X₁ is D; X₂ is S and X₃ is T, such that

(a) the HVR-H1 sequence is GFTFSDSWIH (SEQ ID NO:8);

(b) the HVR-H2 sequence is AWISPYGGSTYYADSVKG (SEQ ID NO:9); and

(c) the HVR-H3 sequence is RHWPGGFDY (SEQ ID NO:10);

In another aspect, the heavy chain polypeptide is further combined with a variable region light chain comprising an HVR-L1, HVR-L2 and HVR-L3, wherein:

(a) the HVR-L1 sequence is RASQX₄X₅X₆TX₇X₈A (SEQ ID NO:12);

(b) the HVR-L2 sequence is SASX₉LX₁₀S, (SEQ ID NO:13); and

(c) the HVR-L3 sequence is QQX₁₁X₁₂X₁₃X₁₄PX₁₅T (SEQ ID NO:14);

wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a still further aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A, such that

(a) the HVR-L1 sequence is RASQDVSTAVA (SEQ ID NO:15);

(b) the HVR-L2 sequence is SASFLYS, (SEQ ID NO:16); and

(c) the HVR-L3 sequence is QQYLYHPAT (SEQ ID NO:17).

Thus, in certain embodiments, an anti-PD-L1 antibody comprises a heavy chain variable region comprising the following HVR-H1, HVR-H2 and HVR-H3 sequences, and comprises a light chain variable region comprising the following HVR-L1, HVR-L2 and HVR-L3 sequences:

(a) the HVR-H1 sequence is GFTFSDSWIH (SEQ ID NO:8);

(b) the HVR-H2 sequence is AWISPYGGSTYYADSVKG (SEQ ID NO:9);

(c) the HVR-H3 sequence is RHWPGGFDY (SEQ ID NO:10);

(d) the HVR-L1 sequence is RASQDVSTAVA (SEQ ID NO:15);

(e) the HVR-L2 sequence is SASFLYS, (SEQ ID NO:16); and

(f) the HVR-L3 sequence is QQYLYHPAT (SEQ ID NO:17)

Trastuzumab-MCC-DM1 (T-DM1)

The present invention includes therapeutic treatments with trastuzumab-MCC-DM1 (T-DM1), an antibody-drug conjugate (CAS Reg. No. 139504-50-0), which has the structure:

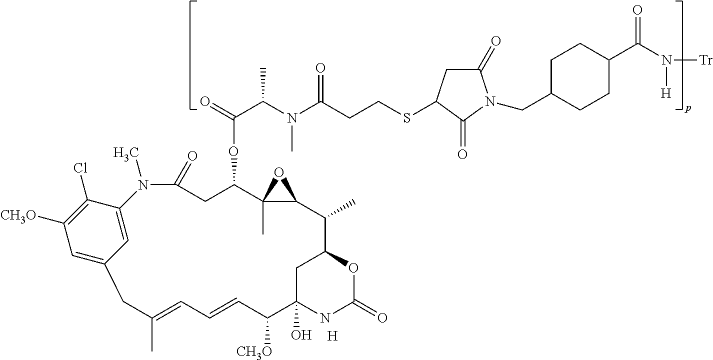

where Tr is trastuzumab linked through linker moiety MCC to the maytansinoid drug moiety DM1 (U.S. Pat. No. 5,208,020; U.S. Pat. No. 6,441,163). The drug to antibody ratio or drug loading is represented by p in the above structure of trastuzumab-MCC-DM1, and ranges in integer values from 1 to about 8. Trastuzumab-MCC-DM1 includes all mixtures of variously loaded and attached antibody-drug conjugates where 1, 2, 3, 4, 5, 6, 7, and 8 drug moieties are covalently attached to the antibody trastuzumab (U.S. Pat. No. 7,097,840; US 2005/0276812; US 2005/0166993). Average drug load is about 3.5.

Trastuzumab can be produced by a mammalian cell (Chinese Hamster Ovary, CHO) suspension culture. The HER2 (or c-erbB2) proto-oncogene encodes a transmembrane receptor protein of 185 kDa, which is structurally related to the epidermal growth factor receptor. Trastuzumab is an antibody that has antigen binding residues of, or derived from, the murine 4D5 antibody (ATCC CRL 10463, deposited with American Type Culture Collection, 12301 Parklawn Drive, Rockville, Md. 20852 under the Budapest Treaty on May 24, 1990). Exemplary humanized 4D5 antibodies include huMAb4D5-1, huMAb4D5-2, huMAb4D5-3, huMAb4D5-4, huMAb4D5-5, huMAb4D5-6, huMAb4D5-7 and huMAb4D5-8 (HERCEPTIN®) as in U.S. Pat. No. 5,821,337.

Trastuzumab-MCC-DM1 may be prepared according to Example 1 of U.S. Application Publication No. 20110165155, for example.

Formulations of Anti-HER2-Maytansinoid Conjugates

Anti-HER2-maytansinoid conjugates, such as trastuzumab-MCC-DM1, may be formulated in accordance with standard pharmaceutical practice for use in a therapeutic combination. The pharmaceutical compositions comprise trastuzumab-MCC-DM1 in association with one or more pharmaceutically acceptable carrier, glidant, diluent, or excipient.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations may be prepared for various routes and types of administration with pharmaceutically acceptable diluents, carriers, excipients or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations of the invention will be dosed and administered in a fashion, i.e., amounts, concentrations, schedules, course, vehicles and route of administration, consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, ethanol, or benzylalcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, including Tween 80, PLURONICS™ or polyethylene glycol (PEG), including PEG400. The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa. Other examples of drug formulations can be found in Liberman, H. A. and Lachman, L., Eds., Pharmaceutical Dosage Forms, Marcel Decker, Vol 3, 2^{nd} Ed., New York, N.Y.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

As a general proposition, the initial pharmaceutically effective amount of trastuzumab-MCC-DM1 administered per dose will be in the range of about 0.3 to 15 mg/kg/day of patient body weight.

A commercial T-DM1 formulation (KADCYLA®, ado-trastuzumab emtansine) is a sterile, white to off-white preservative free lyophilized powder in single-use vials. Each vial contains 100 mg or 160 mg ado-trastuzumab emtansine. Following reconstitution, each single-use vial contains ado-trastuzumab emtansine (20 mg/mL), polysorbate 20 [0.02% (w/v)], sodium succinate (10 mM), and sucrose [6% (w/v)] with a pH of 5.0 and density of 1.026 g/mL. The resulting solution containing 20 mg/mL adotrastuzumab emtansine is administered by intravenous infusion following dilution.

Formulation of Pertuzumab

A commercial formulation of pertuzumab (PERJETA®) contains pertuzumab 420 mg/14 mL (30 mg/mL) in the form of a preservative-free solution for IV infusion.

Administration of Pharmaceutical Compositons

Pharmaceutical compositions described herein may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

Articles of Manufacture

Articles of manufacture, or “kits”, containing anti-HER2-maytansinoid conjugates, such as trastuzumab-MCC-DM1, are useful for the treatment methods herein are provided. In one embodiment, the kit comprises a container comprising trastuzumab-MCC-DM1. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold an anti-HER2-maytansinoid conjugate, such as trastuzumab-MCC-DM1, or a formulation thereof which is effective for use in a treatment method herein, and may have a sterile access port (for example, the container may be an intravenous solution bag or a vial having a stopper pierceable by a hypodermic injection needle). The label or package insert indicates that the composition is used in a treatment method as described and claimed herein. The article of manufacture may also contain a further container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of trastuzumab-MCC-DM1. For example, if the kit comprises a first composition comprising trastuzumab-MCC-DM1 and a second pharmaceutical formulation, the kit may further comprise directions for the simultaneous, sequential or separate administration of the first and second pharmaceutical compositions to a patient in need thereof.

### Examples

In order to illustrate the invention, examples are included as provided in the Figures, the foregoing Brief Description of the Drawings, and the examples below. However, it is to be understood that these examples do not limit the invention and are only meant to suggest a method of practicing the invention.

Abbreviations used in the Examples are listed in the following Table:

| Abbreviation | Definition |
|---|---|
| AC | doxorubicin and cyclophosphamide |
| ADC | antibody-drug conjugate |
| ADCC | antibody-dependent cellular cytotoxicity |
| ATA | anti-therapeutic antibody |
| AUC | area under the concentration-time curve |
| AV | Atrioventricular |
| BCIRG | Breast Cancer International Research Group |
| bpCR | breast pathologic complete response |
| CHF | congestive heart failure |
| CI | confidence interval |
| CL | clearance |
| CNS | central nervous system |
| CRC | colorectal cancer |
| CT | computed tomography |
| CTCAE | Common Terminology Criteria for Adverse Events |
| Cₘₐₓ | maximum serum concentration |
| CMF | cyclophosphamide, methotrexate, and 5-fluorouracil |
| Cₘᵢₙ | minimum serum concentration |
| C_{trough} | trough concentration |
| CYP | Cytochrome |
| DCIS | ductal carcinoma in situ |
| DILI | drug-induced liver injury |
| DFS | disease-free survival |
| DOR | duration of response |
| DRB | Data Review Board |
| RC | Ethics Committee |
| EBC | early breast cancer |
| EBV | Epstein-Barr virus |
| ECHO | Echocardiogram |
| ECOG | Eastern Cooperative Oncology Group |
| eCRF | Electronic Case Report Form |
| EDC | Electronic Data Capture |
| EFS | event-free survival |
| ESMO | European Society for Medical Oncology |
| FFPE | formalin-fixed paraffin-embedded |
| FPI | first patient enrolled |
| GSP | good clinical practice |
| G-CSF | granulocyte-colony stimulating factor |
| HBV | hepatitis B virus |
| HCV | hepatitis C virus |
| HLA | human leukocyte antigen |
| HR | hazard ratio |
| IB | Investogator's Brochure |
| IC | tumor-infiltrating immune cell |
| IDFS | invasive disease-free survival |
| IHC | immunohistochemistry |
| ILD | interstitial lung disease |
| IMC | internal monitoring committee |
| IMP | investigational medicinal product |
| IRF | infusion-related reaction |
| IRR | infusion-related reaction |
| ISH | in situ hybridization |
| IxRS | interactive Web or voice response system |
| LABC | locally advanced breast cancer |
| LCIS | lobular carcinoma in situ |
| LFT | liver function test |
| LPLV | last patient, last visit |
| LVEF | left ventricular ejection fraction |
| LVSD | left ventricular systolic dysfunction |
| MBC | metastatic breast cancer |
| MTD | maximum tolerated dose |
| MUGA | multi-gated acquisition scan |
| NCCN | National Comprehensive Cancer Network |
| NCCTG | North Central Cancer Treatment Group |
| NCI | National Cancer Institute |
| NGS | next-generation sequencing |
| NRH | nodular regenerative hyperplasia |
| NSCLC | non-small cell lung cancer |
| NSABP | National Surgical Adjuvant Breast and Bowel Project |
| NYHA | New Year Heart Association |
| OR | overall response |
| ORR | overall response rate |
| OS | overall survival |
| PI3K | phosphoinositide 3-kinase |
| PBMC | peripheral blood mononuclear cell |
| pCR | pathologic complete response |
| PD-1 | programmed death-1 |
| PD-L1 | programmed death-ligand 1 |
| PFS | progression-free survival |
| PK | pharmacokinetic |
| PO | orally |
| PR | partial response |
| q2w | every 2 weeks |
| q3w | every 3 weeks |
| RCC | renal cell carcinoma |
| RBR | Research Biosample Repository |
| RCR | Roche Clinical Repository |
| RECIST | Response Evaluation Criteria in Solid Tumors |
| SAP | Statistical Analysis Plan |
| SIA | systemic immune activation |
| SIRS | systemic inflammatory response syndrome |
| TC | tumor cell |
| TCH | docetaxel, carboplatin, and trastuzumab |
| TCHP | docetaxel, carboplatin, trastuzumab, and pertuzumab |
| TIL | tumor-infiltrating lymphocyte |
| TSH | thyroid-stimulating hormone |
| tpCR | total pathologic complete response |
| ULN | upper limit of normal |
| WGS | whole genome sequencing |
| Vₛₛ | volume at steady state |

### Example 1—Phase Ib Clinical Study

Objectives

Efficacy Objectives

The primary efficacy objective for this study is to evaluate the safety and tolerability of the following combination treatments administered q3w to patients with HER2-positive MBC or operable LABC or inflammatory EBC:
Atezolizumab in combination with trastuzumab and pertuzumab
Atezolizumab in combination with trastuzumab emtansine

Pharmacokinetic Objectives

The pharmacokinetic (PK) objectives for this study are as follows:
To characterize the pharmacokinetics of atezolizumab, trastuzumab, and pertuzumab when administered concurrently in treatment-naive patients with both metastatic and operable LABC or inflammatory EBC
To characterize the pharmacokinetics of atezolizumab and trastuzumab emtansine when administered concurrently in treatment-naive patients with both metastatic and operable LABC or inflammatory EBC

Exploratory Clinical Activity Objectives

The exploratory clinical activity objectives of this study for patients in the safety-evaluation cohorts are as follows:
To assess the clinical activity of atezolizumab when administered with either trastuzumab and pertuzumab or trastuzumab emtansine in patients with MBC, as measured by PFS, ORR, and duration of objective response (DOR) among patients with an OR per investigator assessment using RECIST v1.1 (E. A. Eisenhauer et al. New response evaluation criteria in solid tumours: Revised RECIST guideline (version 1.1). European Journal of Cancer 45 (2009) 228-247).
To assess the clinical activity of atezolizumab when administered with either trastuzumab and pertuzumab or trastuzumab emtansine in patients with MBC, as measured by PFS, ORR, and DOR per immune-modified RECIST
To assess the clinical activity of atezolizumab when administered with either trastuzumab and pertuzumab or trastuzumab emtansine in patients with operable LABC or inflammatory EBC as measured by the rate of pathologic complete response (pCR; i.e., ypT0/Tis ypN0 in the current AJCC staging system) at breast surgery following completion of neoadjuvant systemic therapy.

Exploratory Biomarker Objectives

The exploratory objectives for this study are as follows:
To identify candidate biomarkers that correlate with safety signals
To assess changes in expression levels of biomarker or biomarker panels during and after investigational treatment with atezolizumab, trastuzumab, and pertuzumab, and atezolizumab plus trastuzumab emtansine

Study Design

Description of Study

This Phase Ib study is an open-label, two-stage study with two active regimens in each stage designed to evaluate the safety and tolerability of combination treatment with atezolizumab, trastuzumab, and pertuzumab or atezolizumab and trastuzumab emtansine in treatment-naive patients with HER2-positive MBC or operable or locally advanced EBC. This study will also address the safety and tolerability of combination treatment with atezolizumab, trastuzumab, and pertuzumab or atezolizumab and trastuzumab emtansine in patients with HER2-positive MBC or treatment-naive patients with operable, or LABC, or inflammatory EBC.

Stage 1 of the study consists of a 3-week safety run-in period for patients with HER2-positive MBC. Two separate cohorts of 3 patients each will be enrolled:
Safety Evaluation Cohort 1A: Patients in Cohort 1A will receive atezolizumab (1200 mg q3w) in combination with trastuzumab (8-mg/kg loading dose, followed by a 6-mg/kg maintenance dose q3w) and pertuzumab (840-mg loading dose, followed by a 420-mg maintenance dose q3w)
Safety Evaluation Cohort 1B: Patients in Cohort 1B will receive atezolizumab (1200 mg q3w) in combination with trastuzumab emtansine (3.6 mg/kg q3w)

If no more than one DLT occurs during the first cycle (3 weeks; the DLT assessment period), another 3 patients will be enrolled into each cohort. If no more than one DLT occurs in 6 patients, the regimen is deemed to have an acceptable safety profile.

Patients who withdraw from study treatment for any reason other than a DLT during this DLT assessment period will not be considered evaluable for DLTs and will be replaced.

During Stage 1 of the study, enrollment within an individual treatment cohort will be stopped if any one of the following stopping criteria according to the NCI CTCAE v4.0 is met during the first two treatment cycles:
Study treatment-related death in at least 1 patient enrolled in the DLT assessment period
DLT in at least 2 patients enrolled in the DLT assessment period: The DLT assessment window will begin with the first cycle of combination treatment with atezolizumab/trastuzumab/pertuzumab or atezolizumab/trastuzumab emtansine and ends 21 days later. Any patient who does not complete the DLT assessment window for any reason other than DLT will be considered non-evaluable for toxicity assessment and will be replaced by an additional patient.

A DLT is defined as one of the following toxicities occurring during the DLT assessment window considered to be possibly, probably, or definitely related to combination treatment:
Anaphylaxis, acute respiratory distress, or Grade 4 IRR
Grade≥4 neutropenia (ANC<500 cells/μL) lasting 7 days
Grade≥3 febrile neutropenia
Grade≥4 thrombocytopenia lasting >48 hours
Any Grade≥3 non-hematologic or non-hepatic major organ adverse event with the following exceptions:
   Grade 3 nausea, vomiting, or diarrhea that resolves to Grade≤1, with or without treatment, prior to the next infusion
   Grade 3 immune-related adverse event that resolves to Grade≤1 with immunosuppressant therapy within 3 weeks of its onset
   Grade 3 autoimmune thyroiditis or other endocrine abnormality that can be managed by endocrine therapy or hormonal replacement
   Grade 3 fever (in the absence of any clinically significant source of fever) that resolves to Grade≤2 within 7 days with supportive care
   Grade≥3 laboratory abnormality that is asymptomatic and deemed by the investigator not to be clinically significant

Any Grade≥3 hepatic toxicity with the following exceptions:

For patients with a Grade 2 AST, ALT, and/or alkaline phosphatase abnormality at baseline, an increase in the baseline abnormality to >10×the ULN will be considered a DLT.

During Stage 1 of the study (the safety run-in phase), the Medical Monitor will review data on an ongoing basis. After 6 patients have either completed Cycle 1 or discontinued because of a toxicity, an internal monitoring committee (IMC) will perform a formal review of the cumulative data and will make appropriate recommendations (e.g., trial will continue as planned, enrollment in one or both arms will be discontinued, amend the protocol, etc.

Metastatic breast cancer patients in the Safety Evaluation Cohorts will continue treatment past Cycle 2 of atezolizumab/trastuzumab/pertuzumab (Cohort 1A) and atezolizumab/trastuzumab emtansine (Cohort 1B) until disease progression or loss clinical benefit (see below) or unacceptable toxicity.

Tumor assessments will be performed every 6 weeks (±7 days) for the first 54 weeks following enrollment, and every 12 weeks (±7 days) thereafter, with additional scans as clinically indicated for patients in Stage 1 of the study. Upon radiographic disease progression according to RECIST v1.1, patients participating in Stage 1 of the study have the option to continue to receive combination treatment until unacceptable toxicity or loss of clinical benefit, provided they meet all of the following criteria:
Evidence of clinical benefit, as assessed by the investigator
Absence of symptoms and signs (including worsening of laboratory values [e.g., new or worsening hypercalcemia]) indicating clinically significant progression of disease
No decline in ECOG performance status that can be attributed to disease progression
Absence of tumor progression at critical anatomical sites (e.g., leptomeningeal disease) that cannot be managed by protocol-specified medical interventions (i.e., pain secondary to disease or unmanageable ascites, etc.), as determined by the investigator after an integrated assessment of radiographic data, biopsy results (if available), and clinical status

After 6 patients in the Safety Evaluation Cohort have been treated in Cohort 1B with atezolizumab/trastuzumab emtansine for at least one cycle without experiencing more than one DLT, a Atezolizumab/Trastuzumab Emtansine Safety Expansion Cohort 2C will begin enrolling patients with HER2-positive MBC that have received prior treatment with trastuzumab and a taxane chemotherapy. Up to 14 additional patients will be enrolled and treated with atezolizumab/trastuzumab emtansine in order to gain additional safety and exploratory clinical activity data to inform potential future investigations of this atezolizumab/trastuzumab emtansine in this patient population.

If 12 patients in Stage 1 of the study (the Safety Evaluation Cohorts; 6 patients in atezolizumab/trastuzumab/pertuzumab arm and 6 patients in the atezolizumab/trastuzumab emtansine arm) have been treated for at least one cycle (3 weeks) without experiencing more than one DLT, then Stage 2 of the Neoadjuvant Window Cohort portion of the study will commence with both Cohorts 2A and 2B enrolling in randomized fashion. Forty patients will be randomized in 1:1 ratio to one of the two treatment cohorts:
Neoadjuvant Window Cohort 2A: Patients in Cohort 2A will receive atezolizumab (1200 mg q3w) in combination with trastuzumab (8-mg/kg loading dose, followed by a 6-mg/kg maintenance dose q3w) and pertuzumab (840-mg loading dose, followed by a 420-mg maintenance dose q3w).
Neoadjuvant Window Cohort 2B: Patients in Cohort 2B will receive atezolizumab (1200 mg q3w) in combination with trastuzumab emtansine (3.6 mg/kg q3w).

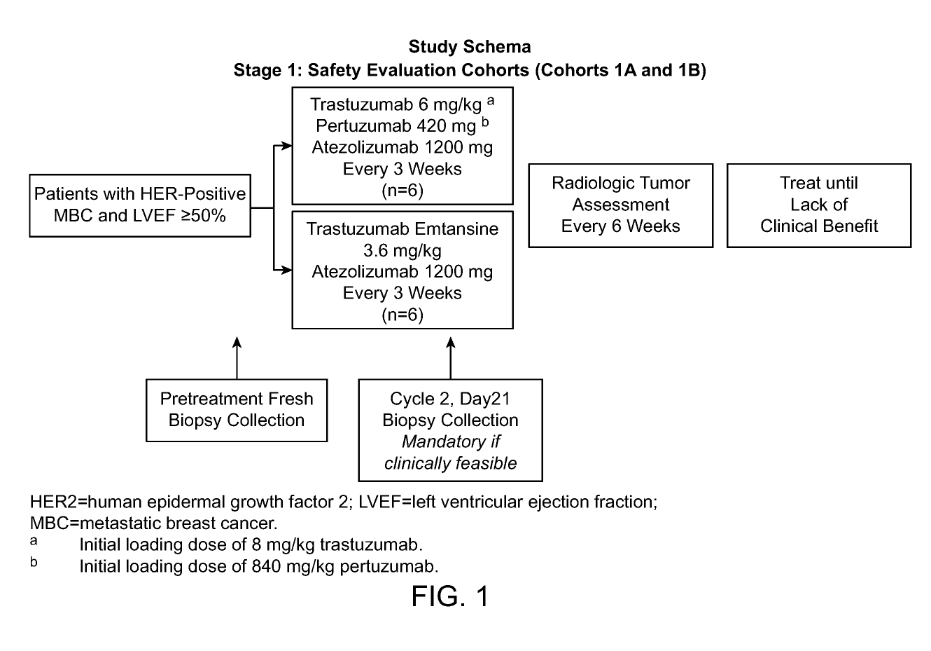

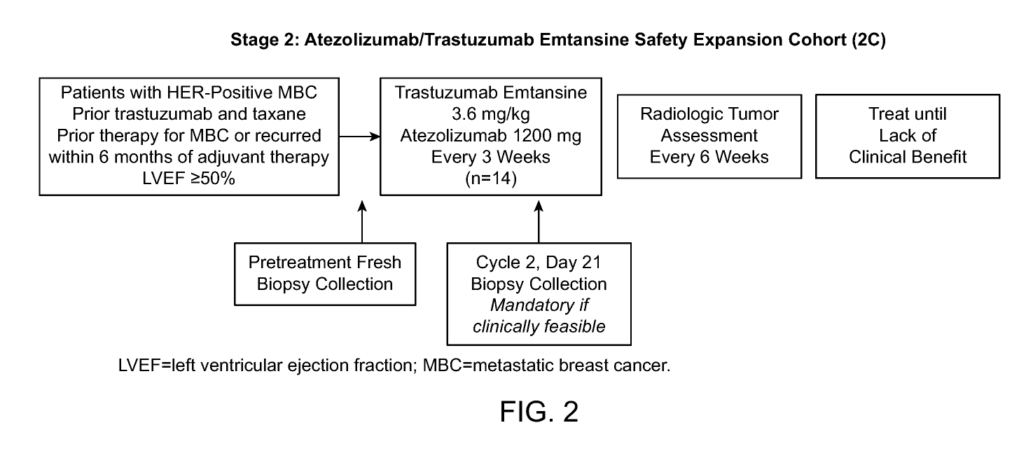

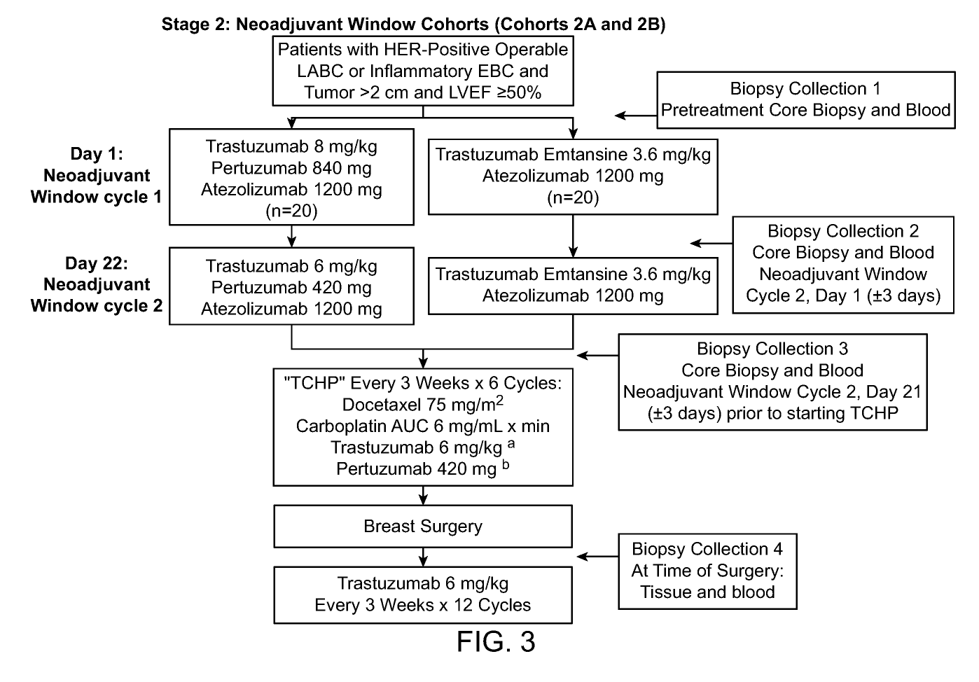

If there is ≤1 DLT in Safety Evaluation Cohort 1A [atezolizumab/trastuzumab/pertuzumab] and >1 DLT in Cohort 1B [atezolizumab/trastuzumab emtansine], then Stage 2 of the Neoadjuvant Window Cohort portion of the study will commence with only Cohort 2A enrolling. Twenty patients will be assigned to treatment as follows (see FIG. 1):
Neoadjuvant Window Cohort 2A: Patients in Cohort 2A will receive atezolizumab (1200 mg q3w) in combination with trastuzumab (8-mg/kg loading dose, followed by a 6-mg/kg maintenance dose q3w) and pertuzumab (840-mg loading dose, followed by a 420 mg maintenance dose q3w).

If there is >1 DLT in Safety Evaluation Cohort 1A [atezolizumab/trastuzumab/pertuzumab] and ≤1 DLT in Cohort 1B [atezolizumab/trastuzumab emtansine], then Stage 2 of the Neoadjuvant Window Cohort portion of the study will commence with only Cohort 2B enrolling. Twenty patients will be assigned to treatment as follows (see FIG. 1):
Neoadjuvant Window Cohort 2B: Patients in Cohort 2B will receive atezolizumab (1200 mg q3w) in combination with trastuzumab emtansine (3.6 mg/kg q3w).

For all patients enrolled in the Neoadjuvant Window Cohort(s) of the study, two cycles of neoadjuvant window therapy will be administered q3w. Mandatory in-breast tumor biopsies and blood collection for biomarker assessment will be performed prior to Cycle 1 and after Cycle 2 of therapy.

Any patient who signs consent for participation in the Stage 2 neoadjuvant window cohorts but does not receive at least one dose of assigned IV therapy with trastuzumab, pertuzumab, and atezolizumab (Cohort 2A) or trastuzumab emtansine and atezolizumab (Cohort 2B) will considered non-evaluable and will be replaced by an additional patient.

For all patients participating in Stage 2 of the study, definitive breast surgery will be performed no earlier than 14 days and no later than 6 weeks following the last infusion of neoadjuvant therapy. The resected breast specimen and all sampled ipsilateral lymph nodes will be evaluated by the local pathologist(s) for pCR. Mandatory tumor samples will be collected at the time of surgery for assessment of tissue biomarkers for response prediction.

Upon the completion of two cycles of neoadjuvant window therapy, all patients will subsequently receive six cycles of neoadjuvant standard-of-care TCHP administered q3w, followed by breast surgery. Upon the completion of surgery, patients will receive 12 cycles of single-agent trastuzumab administered q3w (for a total of 18 cycles of trastuzumab, beginning with the start of standard-of-care TCHP). Radiotherapy and/or endocrine therapy will be administered to patients according to local standard of care.

Number of Patients

Sixty-six patients are anticipated to be enrolled in this study with 12 patients in Stage 1 and 54 patients in Stage 2.

Target Population

Inclusion Criteria for the Safety Evaluation Cohorts

Patients must meet the following criteria for study entry:

Signed Informed Consent Form

Ages≥18 years

Histologically or cytologically documented breast cancer

Metastatic or unresectable locally advanced or recurrent breast cancer

HER2-positive disease documented as in situ hybridization (ISH) positive and/or 3+ by IHC on previously collected tumor tissue and prospectively confirmed by central laboratory testing prior to study enrollment. HER2-positive status will be determined on the basis of archival or, if not available, pre-screening breast biopsy material and defined as an IHC score 3+ and/or positivity by ISH prospectively assessed by central laboratory testing prior to study enrollment. ISH positivity is defined as the ratio of 2 for the number of HER2 gene copies to the number of signals for chromosome 17 copies. Results obtained from central laboratories performing IHC or ISH assays will be used for HER2 eligibility; however, only one positive result is required for eligibility. Only at sites where a legitimate site regulation applies that makes the submittal of blocks unfeasible, and only after having obtained the Sponsor's approval, submittal of different material as described in the study-specific sample manual may be accepted.

Patients with multifocal tumors (i.e., more than one tumor confined to the same quadrant as the primary tumor) are eligible, provided that all discrete lesions are sampled and centrally confirmed as being HER2 positive.

Prior to the start of investigational therapy, a tumor specimen from metastatic or locally advanced disease (if applicable) obtained after the most recent breast cancer systemic therapy, but prior to the start of investigational therapy with atezolizumab, must also be submitted, if clinically feasible. Acceptable samples include core-needle biopsies for deep tumor tissue (minimum, three cores) or excisional, incisional, punch, or forceps biopsies for cutaneous, subcutaneous, or mucosal lesions. Fine-needle aspiration, brushing, cell pellet from pleural effusion, bone metastases, and lavage samples are not acceptable. Tumor tissue from bone metastases is not evaluable for PD-L1 expression and is therefore not acceptable.

Representative archival tumor specimens in paraffin blocks (preferred) or at least 15 unstained slides, with an associated pathology report associated pathology report documenting estrogen receptor status, progesterone receptor status, and HER2 positivity, requested at any time prior to study entry. Only tissue from core needle, punch or excisional biopsy sample collection will be accepted. Fine-needle aspiration, brushing, and lavage samples are not acceptable. For all biopsy types, submitted blocks should have sufficient tissue to generate at least 15 sections, and tissue for which the pathology report specifies that the overall tumor content is low (e.g. “sparse” or “scant”) is not acceptable. Tissue from separate timepoints (such as time of initial diagnosis and time of metastatic diagnosis) or from multiple metastatic tumors may also be collected for a given patient, on the basis of availability. If archival tissue is either insufficient or unavailable, the patient may still be eligible upon discussion with the investigator and Medical Monitor and if the patient can provide at least 10 unstained, serial slides.

ECOG performance status of 0, 1, or 2

LVEF≥50% by either echocardiogram (ECHO) or MUGA

Life expectancy 12 weeks

Measurable disease, as defined by RECIST v1.1 Previously irradiated lesions may be considered as measurable disease only if disease progression has been unequivocally documented at that site since radiation.

Adequate hematologic and end-organ function, as evidenced by the following local laboratory results obtained within 2 weeks prior to the first study treatment (Cycle 1, Day 1):
ANC≥1500 cells/μL, (without granulocyte-colony stimulating factor [G-CSF] support) within 2 weeks prior to Cycle 1, Day 1
Lymphocyte count ≥500 /μL
Platelet count ≥100,000 /μL (without transfusion within 2 weeks prior to Cycle 1, Day 1)
Hemoglobin ≥9.0 g/dL Patients may be transfused or receive erythropoietic treatment to meet this criterion.
AST, ALT, and alkaline phosphatase ≤2.5×the ULN with the following exceptions: Patients with documented liver metastases: AST and ALT≤5×the ULN
Patients with documented liver or bone metastases: alkaline phosphatase ≤5×the ULN
Serum bilirubin ≤1.25×the ULN. Patients with known Gilbert disease who have serum bilirubin level ≤3×the ULN may be enrolled.
INR and aPTT≤1.5×the ULN This applies only to patients who are not receiving therapeutic anticoagulation; patients receiving therapeutic anticoagulation should be on a stable dose.
Calculated CrCl≥30 mL/min

For women who are not postmenopausal (≥12 months of non-therapy-induced amenorrhea) or surgically sterile (absence of ovaries and/or uterus): agreement to remain abstinent (refrain from heterosexual intercourse) or use two adequate methods of contraception, including at least one method with a failure rate of ≤1% per year, during the treatment period and for at least 7 months after the last dose of study drug.
Examples of contraceptive methods with a failure rate of ≤1% per year include bilateral tubal ligation, male sterilization, established, proper use of hormonal contraceptives that inhibit ovulation, hormone-releasing intrauterine devices (IUDs), and copper IUDs.
Periodic abstinence (e.g., calendar, ovulation, symptothermal, or postovulation methods) and withdrawal are not acceptable methods of contraception. The reliability of sexual abstinence needs to be evaluated in relation to the duration of the clinical trial and the preferred and usual lifestyle of the patient.
Barrier methods must always be supplemented with the use of a spermicide.

For men: agreement to remain abstinent (refrain from heterosexual intercourse) or use contraceptive measures and agreement to refrain from donating sperm, as defined below:

With female partners of childbearing potential, men must remain abstinent or use a condom plus an additional contraceptive method that together result in a failure rate of <1% per year during the treatment period and for at least 7 months after the last dose of atezolizumab, trastuzumab emtansine, trastuzumab and/or pertuzumab. Men must refrain from donating sperm during this same period.

With pregnant female partners, men must remain abstinent or use a condom during the treatment period and for at least 7 months after the last dose of atezolizumab, trastuzumab emtansine, trastuzumab and/or pertuzumab.

The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.

Exclusion Criteria for the Safety Evaluation Cohort

Patients who meet any of the following criteria will be excluded from study entry:

Spinal cord compression not definitively treated with surgery and/or radiation, or previously diagnosed and treated spinal cord compression without evidence that disease has been clinically stable for >2 weeks prior to randomization

Known CNS disease, except for treated asymptomatic CNS metastases, provided that all of the following criteria are met:
Measurable disease outside the CNS
Only supratentorial metastases allowed (i.e., no metastases to midbrain, pons, medulla, or spinal cord)
No evidence of progression or hemorrhage after completion of CNS-directed therapy
No ongoing requirement for dexamethasone as therapy for CNS disease (anticonvulsants at a stable dose are allowed)
No stereotactic radiation within 7 days or whole-brain radiation within 14 days prior to randomization

Leptomeningeal Disease

Uncontrolled pleural effusion, pericardial effusion, or ascites. Patients with indwelling catheters (e.g., PleurX®) are allowed.

Uncontrolled tumor-related pain. Patients requiring narcotic pain medication must be on a stable regimen at study entry. Symptomatic lesions (e.g., bone metastases or metastases causing nerve impingement) amenable to palliative radiotherapy should be treated prior to randomization. Patients should have recovered from the effects of radiation. There is no required minimum recovery period. Asymptomatic metastatic lesions whose further growth would likely cause functional deficits or intractable pain (e.g., epidural metastasis that is not presently associated with spinal cord compression) should be considered for loco-regional therapy if appropriate prior to randomization.

Uncontrolled hypercalcemia (>1.5 mmol/L ionized calcium or calcium >12 mg/dL or corrected serum calcium greater than the ULN) or symptomatic hypercalcemia requiring continued use of bisphosphonate therapy. Patients who are receiving denosumab must discontinue use of denosumab and replace it with a bisphosphonate instead while on study. Patients who are receiving bisphosphonate therapy specifically to prevent skeletal events and who do not have a history of clinically significant hypercalcemia are eligible.

History of other malignancy within 5 years prior to randomization, with the exception of those with a negligible risk of metastasis or death and treated with expected curative outcome (such as adequately treated carcinoma in situ of the cervix, basal or squamous cell skin cancer, Stage I uterine cancer, or other cancers with a similar outcome as those mentioned above)

Treatment with any investigational drug within 28 days prior to randomization

Current Grade≥2 peripheral neuropathy (according to the NCI CTCAE v4.0)

Current severe, uncontrolled systemic disease that may interfere with planned treatment (e.g., clinically significant cardiovascular, pulmonary, or metabolic disease, and wound-healing disorders)

Major surgical procedure unrelated to breast cancer or significant traumatic injury within 28 days prior to randomization or anticipation of the need for major surgery during study treatment

Known active liver disease, for example, because of autoimmune hepatic disorders or sclerosis cholangitis

Inclusion Criteria for the Atezolizumab/Trastuzumab Emtansine Safety

Expansion Cohort 2C

Patients must meet the following criteria for study entry:

Signed Informed Consent Form

Ages ≥18 years

Histologically or cytologically documented breast cancer

Metastatic or unresectable locally advanced or recurrent breast cancer

HER2-positive disease documented as in situ hybridization (ISH) positive and/or 3+ by IHC on previously collected tumor tissue and prospectively confirmed by central laboratory testing prior to study enrollment. HER2-positive status will be determined on the basis of archival or, if not available, pre-screening breast biopsy material and defined as an IHC score 3+ and/or positivity by ISH prospectively assessed by central laboratory testing prior to study enrollment. ISH positivity is defined as the ratio of ≥2 for the number of HER2 gene copies to the number of signals for chromosome 17 copies. Results obtained from central laboratories performing IHC or ISH assays will be used for HER2 eligibility; however, only one positive result is required for eligibility. Only at sites where a legitimate site regulation applies that makes the submittal of blocks unfeasible, and only after having obtained the Sponsor's approval, submittal of different material as described in the study-specific sample manual may be accepted.

Patients with multifocal tumors (i.e., more than one tumor confined to the same quadrant as the primary tumor) are eligible, provided that all discrete lesions are sampled and centrally confirmed as being HER2 positive.

Prior treatment for breast cancer in the neoadjuvant, adjuvant, locally advanced, or recurrent/metastatic setting that must include both a taxane, alone or in combination with another agent, and trastuzumab, alone or in combination with another agent, in the adjuvant, unresectable, locally advanced, or metastatic setting.

Documented progression of incurable, unresectable, locally advanced, or metastatic breast cancer, as defined by the investigator.
Progression must occur during or after the most recent treatment for locally advanced unresectable/MBC or within 6 months after completing adjuvant therapy.

Prior to the start of investigational therapy, a tumor specimen from metastatic or locally advanced disease (if applicable) obtained after the most recent breast cancer systemic therapy, but prior to the start of investigational therapy with atezolizumab, must also be submitted, if clinically feasible.
Acceptable samples include core-needle biopsies for deep tumor tissue (minimum, three cores) or excisional, incisional, punch, or forceps biopsies for cutaneous, subcutaneous, or mucosal lesions.
Fine-needle aspiration, brushing, cell pellet from pleural effusion, bone metastases, and lavage samples are not acceptable.
Tumor tissue from bone metastases is not evaluable for PD-L1 expression and is therefore not acceptable.

Representative archival tumor specimens in paraffin blocks (preferred) or at least 15 unstained slides, with an associated pathology report documenting estrogen receptor status, progesterone receptor status, and HER2 positivity, requested at any time prior to study entry. Only tissue from core needle, punch, or excisional biopsy sample collection will be accepted. Fine-needle aspiration, brushing, and lavage samples are not acceptable. For all biopsy types, submitted blocks should have sufficient tissue to generate at least 15 sections, and tissue for which the pathology report specifies that the overall tumor content is low (e.g. “sparse” or “scant”) is not acceptable. Tissue from separate timepoints (such as time of initial diagnosis and time of metastatic diagnosis) or from multiple metastatic tumors may also be collected for a given patient, on the basis of availability. If archival tissue is either insufficient or unavailable, the patient may still be eligible upon discussion with the investigator and Medical Monitor and if the patient can provide at least 10 unstained, serial slides.

ECOG performance status of 0, 1, or 2

LVEF≥50% by either echocardiogram (ECHO) or MUGA

Life expectancy ≥12 weeks

Measurable disease, as defined by RECIST v1.1. Previously irradiated lesions may be considered as measurable disease only if disease progression has been unequivocally documented at that site since radiation.

Adequate hematologic and end-organ function, as evidenced by the following local laboratory results obtained within 2 weeks prior to the first study treatment (Cycle 1, Day 1):
ANC≥1500 cells/μL, (without granulocyte-colony stimulating factor [G-CSF] support) within 2 weeks prior to Cycle 1, Day 1
Lymphocyte count ≥500 /μL
Platelet count ≥100,000 /μL (without transfusion within 2 weeks prior to Cycle 1, Day 1)
Hemoglobin ≥9.0 g/dL Patients may be transfused or receive erythropoietic treatment to meet this criterion.
AST, ALT, and alkaline phosphatase ≤2.5×the ULN with the following exceptions: Patients with documented liver metastases: AST and ALT≤5×the ULN

Patients with documented liver or bone metastases: alkaline phosphatase 5×the ULN

Serum bilirubin ≤1.25×the ULN Patients with known Gilbert disease who have serum bilirubin level ≤3×the ULN may be enrolled.

INR and aPTT≤1.5×the ULN This applies only to patients who are not receiving therapeutic anticoagulation; patients receiving therapeutic anticoagulation should be on a stable dose.

Calculated CrCl≥30 mL/min

For women who are not postmenopausal (≥12 months of non-therapy-induced amenorrhea) or surgically sterile (absence of ovaries and/or uterus): agreement to remain abstinent (refrain from heterosexual intercourse) or use two adequate methods of contraception, including at least one method with a failure rate of ≤1% per year, during the treatment period and for at least 7 months after the last dose of study drug.
Examples of contraceptive methods with a failure rate of ≤1% per year include bilateral tubal ligation, male sterilization, established, proper use of hormonal contraceptives that inhibit ovulation, hormone-releasing intrauterine devices (IUDs), and copper IUDs.
Periodic abstinence (e.g., calendar, ovulation, symptothermal, or postovulation methods) and withdrawal are not acceptable methods of contraception. The reliability of sexual abstinence needs to be evaluated in relation to the duration of the clinical trial and the preferred and usual lifestyle of the patient.
Barrier methods must always be supplemented with the use of a spermicide.

For men: agreement to remain abstinent (refrain from heterosexual intercourse) or use contraceptive measures and agreement to refrain from donating sperm, as defined below.
With female partners of childbearing potential, men must remain abstinent or use a condom plus an additional contraceptive method that together result in a failure rate of <1% per year during the treatment period and for at least 7 months after the last dose of atezolizumab, trastuzumab emtansine, trastuzumab, and/or pertuzumab. Men must refrain from donating sperm during this same period.
With pregnant female partners, men must remain abstinent or use a condom during the treatment period and for at least 7 months after the last dose of atezolizumab, trastuzumab emtansine, trastuzumab, and/or pertuzumab.
The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.

Exclusion Criteria for the Atezolizumab/Trastuzumab Emtansine Safety

Expansion Cohort 2C

Patients who meet any of the following criteria will be excluded from study entry:

Spinal cord compression not definitively treated with surgery and/or radiation, or previously diagnosed and treated spinal cord compression without evidence that disease has been clinically stable for >2 weeks prior to randomization

Known CNS disease, except for treated asymptomatic CNS metastases, provided that all of the following criteria are met:
Measurable disease outside the CNS
Only supratentorial metastases allowed (i.e., no metastases to midbrain, pons, medulla, or spinal cord)
No evidence of progression or hemorrhage after completion of CNS-directed therapy
No ongoing requirement for dexamethasone as therapy for CNS disease (anticonvulsants at a stable dose are allowed)
No stereotactic radiation within 7 days or whole-brain radiation within 14 days prior to randomization

Leptomeningeal Disease

Uncontrolled pleural effusion, pericardial effusion, or ascites Patients with indwelling catheters (e.g., PleurX®) are allowed.

Uncontrolled tumor-related pain Patients requiring narcotic pain medication must be on a stable regimen at study entry. Symptomatic lesions (e.g., bone metastases or metastases causing nerve impingement) amenable to palliative radiotherapy should be treated prior to randomization. Patients should have recovered from the effects of radiation. There is no required minimum recovery period. Asymptomatic metastatic lesions whose further growth would likely cause functional deficits or intractable pain (e.g., epidural metastasis that is not presently associated with spinal cord compression) should be considered for loco-regional therapy if appropriate prior to randomization.

Uncontrolled hypercalcemia (>1.5 mmol/L ionized calcium or calcium >12 mg/dL or corrected serum calcium greater than the ULN) or symptomatic hypercalcemia requiring continued use of bisphosphonate therapy. Patients who are receiving denosumab must discontinue use of denosumab and replace it with a bisphosphonate instead while on study. Patients who are receiving bisphosphonate therapy specifically to prevent skeletal events and who do not have a history of clinically significant hypercalcemia are eligible.

History of other malignancy within 5 years prior to randomization, with the exception of those with a negligible risk of metastasis or death and treated with expected curative outcome (such as adequately treated carcinoma in situ of the cervix, basal or squamous cell skin cancer, Stage I uterine cancer, or other cancers with a similar outcome as those mentioned above)

Treatment with any investigational drug within 28 days prior to randomization

Current Grade≥2 peripheral neuropathy (according to the NCI CTCAE v4.0)

Current severe, uncontrolled systemic disease that may interfere with planned treatment (e.g., clinically significant cardiovascular, pulmonary, or metabolic disease, and wound-healing disorders)

Major surgical procedure unrelated to breast cancer or significant traumatic injury within 28 days prior to randomization or anticipation of the need for major surgery during study treatment

Known active liver disease, for example, because of autoimmune hepatic disorders or sclerosis cholangitis

Inclusion Criteria for the Neoadjuvant Window Cohorts

Patients must meet the following criteria for study entry:

Signed Informed Consent Form

Histologically confirmed invasive breast cancer with a primary tumor size >2 cm by at least one radiographic or clinical measurement

HER2-positive breast cancer HER2-positive status will be determined on the basis of archival breast biopsy material and defined as an IHC score 3+ and/or positivity by ISH prospectively assessed by central laboratory testing prior to study enrollment. ISH positivity is defined as the ratio of ≥2 for the number of HER2 gene copies to the number of signals for chromosome 17 copies. Results obtained from central laboratories performing IHC or ISH assays will be used to establish HER2 eligibility; however, only one positive result is required for eligibility. Only at sites where a legitimate site regulation applies that makes the submittal of blocks unfeasible, and only after having obtained the Sponsor's approval, submittal of different material as described in the study-specific sample manual may be accepted. Patients with multifocal tumors (i.e., more than one tumor confined to the same quadrant as the primary tumor) are eligible, provided that all discrete lesions are sampled and centrally confirmed as being HER2 positive.

Human leukocyte antigen (HLA)-A2 haplotype Patients enrolled in the neoadjuvant cohort should be HLA-A2 positive. HLA-A2 haplotype will be determined on the basis of a dual methodology approach using DNA from cheek swabs. A central laboratory will perform Sanger Sequencing (SBT) supplemented by truncated version of SSOP (sequence-specific oligonucleotide probe) to confirm concordance of serological group assignments. Both assays are polymerase chain reaction (PCR) molecular based. The cheek swab (also called buccal swab) test is a long cotton swab that is rubbed inside the inner cheek lining of a person's mouth. Typically, four swabs are collected, and DNA is extracted from this material. Buccal swabs are easy and painless to collect.

Stage at presentation: cT2-cT4, cN0-cN3, or cM0

Known hormone receptor status of the primary tumor: Hormone receptor-positive status may be determined by either known positive estrogen receptor and known negative progesterone receptor status

Patient agreement to undergo mastectomy or breast-conserving surgery after neoadjuvant therapy

Willingness and ability to comply with scheduled visits, treatment plans, laboratory tests, and other study procedures, including serial core biopsies of in-breast tumor during the investigational treatment phase of the study

Ages ≥18 years

ECOG performance status of 0, 1, or 2

Adequate hematologic and end-organ function during screening (within 7 days before the first dose of study drug), as evidenced by the following laboratory results:
ANC≥1500 cells/μL,
Platelet count ≥100,000 cells/μL
Hemoglobin ≥9.0 g/dL Patients may receive transfused RBCs to achieve this level.
AST, ALT, and alkaline phosphatase ULN
Serum bilirubin ≤the ULN Patients with known Gilbert disease may be enrolled, but direct bilirubin should be within the normal range.
INR and aPTT≤1.5×the ULN This only applies to patients who are not receiving therapeutic anticoagulation; patients receiving therapeutic anticoagulation should be on a stable dose.
Calculated CrCl≥30 mL/min

Baseline LVEF≥50% measurement on ECHO or MUGA scan

Negative results of serum pregnancy test for premenopausal women of reproductive capacity and for women <12 months after entering menopause

For women who are not post-menopausal 12 months of non-therapy-induced amenorrhea) or surgically sterile (absence of ovaries and/or uterus): agreement to remain abstinent (refrain from heterosexual intercourse) or use two adequate methods of contraception, including at least one method with a failure rate of 1% per year during the treatment period and for at least 7 months after the last dose of study drug
Examples of contraceptive methods with a failure rate of 1% per year include bilateral tubal ligation, male sterilization, established, proper use of hormonal contraceptives that inhibit ovulation, hormone-releasing IUDs, and copper IUDs.
Periodic abstinence (e.g., calendar, ovulation, symptothermal, or post-ovulation methods) and withdrawal are not acceptable methods of contraception. The reliability of sexual abstinence needs to be evaluated in relation to the duration of the clinical trial and the preferred and usual lifestyle of the patient.
Barrier methods must always be supplemented with the use of a spermicide.

For men: agreement to remain abstinent or use a condom plus an additional contraceptive method that together result in a failure rate of <1% per year during the treatment period and for at least 7 months plus 74 days (a spermatogenesis cycle) after the last dose of study drug and agreement to refrain from donating sperm during this same period. Men with a pregnant partner must agree to remain abstinent or use a condom for the duration of the pregnancy. Abstinence is only acceptable if it is in line with the preferred and usual lifestyle of the patient. Periodic abstinence (e.g., calendar, ovulation, symptothermal, or post-ovulation methods) and withdrawal are not acceptable methods of contraception.

Exclusion Criteria for the Neoadjuvant Window Cohorts

Patients who meet any of the following criteria will be excluded from study entry:

Stage IV (metastatic) breast cancer

Prior systemic therapy for treatment and prevention of breast cancer

History of DCIS, except for patients treated exclusively with mastectomy >5 years prior to diagnosis of current breast cancer

History of pleomorphic LCIS, except for patients surgically managed >5 years prior to diagnosis of current breast cancer Note: Patients with non-pleomorphic LCIS (either untreated or treated with surgery) are eligible.

Patients with multicentric (i.e., multiple tumors involving more than one quadrant) breast cancer

Patients with bilateral breast cancer

Previous incisional and/or excisional biopsy of the primary tumor and/or axillary lymph nodes

Axillary lymph node dissection prior to initiation of neoadjuvant therapy Patients with clinically negative axilla (by physical examination and radiographic imaging) may undergo a sentinel lymph node procedure prior to neoadjuvant therapy if in keeping with local practice.

History of concurrent or previously treated non-breast-related malignancies except for appropriately treated non-melanoma skin cancer and/or in situ carcinomas, including cervix, colon, and skin A patient with previously invasive non-breast-related cancer is eligible, provided that he or she has been disease free for more than 5 years.

Treatment with any investigational drug within 28 days prior to randomization

Current Grade≥2 peripheral neuropathy (according to the NCI CTCAE v4.0)

Current severe, uncontrolled systemic disease that may interfere with planned treatment (e.g., clinically significant cardiovascular, pulmonary, or metabolic disease, and wound-healing disorders)

Major surgical procedure unrelated to breast cancer or significant traumatic injury within 28 days prior to randomization or anticipation of the need for major surgery during study treatment

Known active liver disease, for example, due to autoimmune hepatic disorders or sclerosis cholangitis

General Medical Exclusion Criteria for All Patients

Patients meeting any of the following criteria will be excluded from the study:

Pregnancy or Lactation

Evidence of significant uncontrolled concomitant disease that could affect compliance with the protocol or interpretation of results, including significant liver disease (such as cirrhosis), uncontrolled major seizure disorder, or superior vena cava syndrome

Severe infection within 4 weeks prior to randomization, including, but not limited to, hospitalization for complications of infection, bacteremia, or severe pneumonia

Major surgical procedure within 4 weeks prior to randomization or anticipation of the need for a major surgical procedure during the study other than for diagnosis

Placement of central venous access catheter(s) (e.g., port or similar) is not considered a major surgical procedure and is therefore permitted.

Exclusion Criteria Related to Atezolizumab, Trastuzumab Emtansine,

Trastuzumab, and Pertuzumab for All Patients

Patients who meet any of the following criteria will be excluded from study entry:

History of severe allergic, anaphylactic, or other hypersensitivity reactions to chimeric or humanized antibodies or fusion proteins

Known hypersensitivity or allergy to biopharmaceuticals produced in Chinese hamster ovary cells or any component of the atezolizumab, trastuzumab emtansine, trastuzumab, and/or pertuzumab formulations

History of autoimmune disease, including, but not limited to, myasthenia gravis, autoimmune myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barré syndrome, multiple sclerosis, vasculitis, or glomerulonephritis Patients with a history of autoimmune-related hypothyroidism on a stable dose of thyroid replacement hormone may be eligible for this study. Patients with controlled Type 1 diabetes mellitus on a stable insulin regimen may be eligible for this study. Patients with eczema, psoriasis, lichen simplex chronicus of vitiligo with dermatologic manifestations only (e.g., patients with psoriatic arthritis would be excluded) are eligible, provided that they meet the following conditions:

Patients with psoriasis must have a baseline ophthalmologic exam to rule out ocular manifestations.
Rash must cover <10% of BSA.
Disease is well controlled at baseline and requiring only low-potency topical steroids (e.g., hydrocortisone 2.5%, hydrocortisone butyrate 0.1%, flucinolone 0.01%, desonide 0.05%, aclometasone dipropionate 0.05%)
No acute exacerbations of underlying condition within the last 12 months (not requiring PUVA [psoralen plus ultraviolet A radiation], methotrexate, retinoids, biologic agents, oral calcineurin inhibitors, or high-potency or oral steroids)

Prior allogeneic stem cell or solid organ transplantation

History of idiopathic pulmonary fibrosis (including pneumonitis), drug-induced pneumonitis, organizing pneumonia (i.e., bronchiolitis obliterans, cryptogenic organizing pneumonia), or evidence of active pneumonitis on screening chest CT scan Patients with a history of radiation pneumonitis in the radiation field (fibrosis) are permitted.

Positive Test for HIV

Active hepatitis B (defined as having a positive HBsAg test at screening) or hepatitis C Patients with previous hepatitis B virus (HBV) infection or resolved HBV infection (defined as having a negative HBsAg test and a positive antibody to hepatitis B core antigen [anti-HBc] antibody test) are eligible. Patients positive for hepatitis C virus (HCV) antibody are eligible only if PCR is negative for HCV RNA.

Active Tuberculosis

Receipt of a live, attenuated vaccine within 4 weeks prior to randomization or anticipation that such a live, attenuated vaccine will be required during the study

Prior treatment with CD137 agonists, anti-PD-1, or anti-PD-L1 therapeutic antibody or pathway-targeting agents

Treatment with systemic immunostimulatory agents (including, but not limited to, interferons or IL-2) within 4 weeks or five half-lives of the drug (whichever is shorter) prior to randomization

Treatment with systemic corticosteroids or other systemic immunosuppressive medications (including but not limited to prednisone, dexamethasone, cyclophosphamide, azathioprine, methotrexate, thalidomide, and anti-TNF agents) within 2 weeks prior to randomization, or anticipated requirement for systemic immunosuppressive medications during the trial Patients who have received acute, low-dose, systemic immunosuppressant medications (e.g., a one-time dose of dexamethasone for nausea) may be enrolled in the study.

History of exposure to the following cumulative doses of anthracycline as specified below:
Doxorubicin >500 mg/m²
Liposomal doxorubicin >500 mg/m²
Epirubicin >720 mg/m²
Mitoxantrone >120 mg/m²
Idarubicin >90 mg/m²

If another anthracycline or more than one anthracycline has been used, then the cumulative dose must not exceed the equivalent of 500 mg/m² of doxorubicin.

Cardiopulmonary dysfunction, defined as:
Uncontrolled hypertension (systolic blood pressure >150 mmHg and/or diastrolic blood pressure >100 mmHg)
Inadequately controlled angina or serious cardiac arrhythmia not controlled by adequate medication
Inadequate LVEF at baseline, defined as LVEF<50% by either ECHO or MUGA
History of symptomatic CHF—Grade≥3 per NCI CTCAE v 4.0 or NYHA Class ≥II
History of a decrease in LVEF to <40% or symptomatic CHF with prior trastuzumab treatment
Myocardial infarction within 6 months prior to randomization
Current dyspnea at rest due to complications of advanced malignancy, or other disease requiring oxygen therapy

Length of Study

The planned duration of this trial is approximately 25 months.

End of Study

The end of the study is defined as the date when the last patient, last visit (LPLV) occurs. LPLV is expected to occur approximately 52 weeks after the last patient is enrolled.

Outcome Measures

Safety Outcome Measures

The safety and tolerability of atezolizumab administered in combination with trastuzumab plus pertuzumab and trastuzumab emtansine to patients with HER2-positive MBC and treatment-naive patients with operable LABC or inflammatory EBC will be assessed using the following primary safety outcome measures:

Incidence and nature of DLTs

Incidence, nature, and severity of adverse events graded according to NCI CTCAE v4.0

Secondary Safety Outcome Measures

Additionally, safety will be assessed using the following secondary safety outcome measures:
Incidence of ATA response and the potential correlation with PK, PD, and safety parameters
Changes in vital signs and ECG parameters
Changes in clinical laboratory results
Number of cycles and dose intensity

Cardiac and Hepatic Safety Outcome Measures

The cardiac and hepatic safety measures for this study are as follows:
Cardiac events, defined as death from cardiac cause or severe CHF (NYHA Class III or IV) with a decrease of ≥10-percentage points in LVEF from baseline to an LVEF of <50%
Hepatic events, defined as death from hepatic cause, severe drug-induced liver injury

(DILI), confirmed Hy's law cases, or nodular regenerative hyperplasia (NRH)

Pharmacokinetic Outcome Measures

The PK outcome measures for this study are as follows:
Maximum observed serum atezolizumab concentration (Cₘₐₓ) after infusion on Day 1 of Cycle 1
Minimum observed serum atezolizumab, trastuzumab, trastuzumab emtansine, and pertuzumab concentrations (Cₘᵢₙ) for each cycle at which pharmacokinetics are measured.
Other parameters, such as accumulation ratio, half-life, and dose proportionality, may also be calculated.

Exploratory Outcome Measures

The clinical activity of atezolizumab administered in combination with trastuzumab/pertuzumab or trastuzumab emtansine to HER2-positive patients with MBC will be assessed as follows:
Investigator-assessed PFS using RECIST v1.1
ORR, defined as the percentage of patients with an objective tumor response (either PR or CR) per investigator assessment using RECIST v1.1
DOR, defined as the time from the first occurrence of a documented objective tumor response to the time of radiographic progression (according to investigator assessment using RECIST v1.1) or death from any cause on study, whichever occurs first
PFS, ORR, and DOR per investigator assessment using immune-modified RECIST
OS, defined as the time from the first dose of study drug to death

The clinical activity of atezolizumab administered in combination with trastuzumab/pertuzumab or trastuzumab emtansine to HER2-positive patients with operable LABC or inflammatory EBC will be assessed as follows:
Rate of pCR (ypT0/Tis ypN0 in the current AJCC staging system) at breast surgery following completion of neoadjuvant systemic therapy per local pathology evaluation.

The exploratory outcome measures for patients with MBC in the safety-evaluation and the safety expansion cohorts of this study are as follows:
Association of tumor immune-related exploratory biomarkers in serially obtained biopsies of tumor tissue with disease status and/or response to atezolizumab administered to patients in combination with trastuzumab plus pertuzumab or trastuzumab emtansine

The exploratory outcome measures for treatment-naive patients with operable LABC or inflammatory EBC in the neoadjuvant cohorts of this study are as follows:
Association of tumor immune-related exploratory biomarkers in serially obtained biopsies of tumor tissue with disease status and/or response to atezolizumab administered to patients in combination with trastuzumab plus pertuzumab and trastuzumab emtansine. Assessment over time of exploratory biomarkers in plasma and whole blood (including, but not limited to, cytokines, such as IL-18) collected before and after two cycles of investigational window treatment with atezolizumab administered to patients in combination with trastuzumab plus pertuzumab and trastuzumab emtansine and after standard-of-care therapy with TCHP followed by surgery

Investigational Medicinal Products

Test Product (Investigational Drug)
Atezolizumab 1200 mg flat dose administered via IV infusion on Day 1 of every 21-day cycle
Trastuzumab emtansine 3.6 mg/kg administered via IV infusion on Day 1 of every 21-day cycle
Trastuzumab 8 mg/kg loading dose, then 6 mg/kg administered via IV infusion on Day 1 of every 21-day cycle
Pertuzumab 840 mg loading dose then 420 mg administered via IV infusion on Day 1 of every 21-day cycle

Non-Investigational Medicinal Products
Carboplatin AUC 6 mg/mL×min administered via IV infusion on Day 1 every 21-days for 6 cycles
Docetaxel 75 mg/m2 administered via IV infusion on Day 1 every 21 days for 6 cycles

Statistical Methods

Primary Analysis

The primary analysis will be based on patient data collected through study discontinuation or at the end of study. All analyses will be conducted using the safety-evaluable population, defined as all patients who receive any amount of study drugs.

Data will be described and summarized as warranted by sample size. That is, listings may be used in lieu of tables in the event of small sample size.

No formal hypothesis testing is planned. Descriptive statistics will be used to summarize the safety and clinical activity of treatment regimens. Continuous variables will be summarized using mean, standard deviation, median, and range; categorical variables will be summarized using count and percentage. All summaries will be presented by stage and treatment cohort.

Determination of Sample Size

Design considerations were not made with regard to explicit power and the type I error but to obtain preliminary safety, PK, and PD information of atezolizumab in combination with trastuzumab plus pertuzumab or trastuzumab emtansine in patients with HER2-positive breast cancer.

Sixty-six patients are anticipated to be enrolled in this study with 12 patients in Stage 1 and 54 patients in Stage 2.

In Stage 1 of the study, the safety-evaluation phase, the study plans to enroll 12 patients with HER2-positive MBC in each of the two treatment cohorts (atezolizumab, trastuzumab, and pertuzumab or atezolizumab and trastuzumab emtansine), with 6 patients in each cohort. No more than 1 patient out of 6 patients can have a DLT in Stage 1 before the respective Stage 2 treatment cohort is allowed to open. With an assumption of true DLT rate at 10%, 15%, or 20%, the probability of observing no more than 1 DLT in Stage 1 is 88.6%, 77.6%, and 65.5%, respectively. If these two regimens are deemed to have an acceptable safety profile, another 40 patients with HER2-positive LABC or inflammatory EBC will be randomized at 1:1 ratio to these treatment cohorts in Stage 2 in the neoadjuvant setting. If only one cohort in Stage 1 is deemed to have an acceptable safety profile, another 20 patients will be enrolled in the respective treatment arm in the new neoadjuvant window cohort. Based on the assumption that all patients in each arm (n=20) have evaluable paired data for biopsy, a 30% increase in intratumoral CD8 T cells, considered biologically meaningful, has an 80% 2-sided confidence interval (0.181, 0.419). This assumes a standard deviation of 40%. In addition, 14 patients will be enrolled in the Atezolizumab/Trastuzumab Emtansine Safety Expansion Cohort 2C. For a given adverse event with a true rate of 10%, 5%, or 1%, the probability of observing at least one such adverse event in the safety expansion cohort of 14 patients will be 77.1%, 51.2%, and 13.1%, respectively.

### Example 2—Phase Ib Clinical Study—Dosage, Administration and Compliance

In the Phase Ib clinical study described in Example 1, atezolizumab, trastuzumab emtansine, trastuzumab, and pertuzumab will be labeled according to regulatory requirements in each country, as well as in accordance with International Conference of Harmonisation (ICH) Good Clinical Practice (GCP) and will be labeled for investigational use only. The Sponsor will provide atezolizumab, trastuzumab emtansine, trastuzumab, and pertuzumab free of charge to all study sites.

For contraindications, adverse reactions, warnings and precautions for atezolizumab, trastuzumab emtansine, trastuzumab and pertuzumab, refer to each agent's Investigator Brochures.

For contraindications, adverse reactions, warnings, and precautions for docetaxel and carboplatin, refer to the national prescribing information.

Atezolizumab

Dose

Patients will receive 1200 mg of atezolizumab administered by IV infusion q3w in a monitored setting where there is immediate access to trained personnel and adequate equipment and medicine to manage potentially serious reactions. No dilution of the vial contents is required.

Atezolizumab will be delivered in 250-mL 0.9% NaCl IV infusion bags with product contacting surfaces of polyvinyl chloride (PVC) or polyolefin and IV infusion lines with product contacting surfaces of PVC or polyethylene and 0.2-μm in-line filters (filter membrane of polyethersulfone). Atezolizumab is compatible with these infusion materials (bags and infusion lines).

Administration

Atezolizumab infusions will be administered according to the instructions outlined in Table 1.

Trastuzumab

For information on the formulation, packaging, and handling of trastuzumab, see the most recent version of the Trastuzumab Investigetor's Brochure as well as local prescribing information.

Dose

For Patients Enrolled in Cohort 1A (Trastuzumab, Pertuzumab, and Atezolizumab) of the Safety-Evaluation Phase:

Trastuzumab will be administered by IV infusion at a loading dose of 8 mg/kg, followed by doses of 6 mg/kg q3w.

For Patients Enrolled in Cohort 2A (Trastuzumab, Pertuzumab, and Atezolizumab) of the Neoadjuvant Window Phase:

Trastuzumab will be administered by IV infusion at a loading dose of 8 mg/kg, followed by doses of 6 mg/kg q3w. Trastuzumab will be administered for two neoadjuvant window cycles as an IV infusion every 21 (±3) days for patients in Cohort 2A (trastuzumab+pertuzumab+atezolizumab).

After the completion of the two neoadjuvant window cycles, patients in Cohort 2A will receive standard-of-care treatment with TCHP for six cycles every 21 (±3) days followed by breast surgery. Starting 3 weeks after surgery, patients will begin adjuvant therapy with trastuzumab, which will be administrated at a dose of 6 mg/kg every 21 (±3) days for a maximum of 18 total cycles of standard-of-care HER2-directed therapy (i.e., 6 cycles of TCHP+12 cycles of single-agent adjuvant trastuzumab).

For Patients Enrolled in Cohort 2B (Trastuzumab Emtansine and Atezolizumab) of the Neoadjuvant Window Phase:

After completion of the two neoadjuvant window cycles with trastuzumab emtansine, patients in Cohort 2B will receive standard-of-care treatment with TCHP for six cycles every 21 (±3) days. For Cycle 1 of TCHP, trastuzumab will be administered to patients as an IV infusion at a loading dose of 8 mg/kg. Subsequent doses of 6 mg/kg trastuzumab will be administered every 21 (±3) days. Starting 3 weeks after surgery, patients will begin adjuvant therapy with trastuzumab, which will be administrated 6 mg/kg every 21 (±3) days to complete a maximum of 18 total cycles of standard-of-care HER2-directed therapy (6 cycles of TCHP+12 cycles of single-agent adjuvant trastuzumab).

For All Patients:

The dose of trastuzumab will be calculated on the basis of the patient's baseline weight. Weight will be measured at each visit. If there is a 10% change in weight compared with baseline the dose should be adjusted accordingly. The dose must be re-adjusted for additional weight changes 10%.

Administration may be delayed to assess or treat adverse events such as cardiac adverse events, but no dose reduction will be allowed.

Administration

Please refer to the pertuzumab Investogator's Brochure and Table 2 (Safety Evaluation Cohort 1A), Table 4 (Naoadjuvant Window Cohort 2A), and Table 5 (Neoadjuvant Window Cohort 2B).

Pertuzumab

For information on the formulation, packing, and handling of pertuzumab, see the most recent version of the pertuzumab Investigator's Brochure as well as local prescribing information.

Dose

For Patients Enrolled in Cohort 1A (Trastuzumab, Pertuzumab, and Atezolizumab) in the Safety Evaluation Stage:

Pertuzumab will be administered by IV infusion at a fixed total loading dose of 840 mg for the first cycle, followed by a fixed dose of 420 mg for subsequent cycles.

For Patients Enrolled in Cohort 2A (Trastuzumab, Pertuzumab, and Atezolizumab) in the Neoadjuvant Window Phase:

Pertuzumab will be administered by IV infusion every 21 (±3) days as a fixed total loading dose of 840 mg for the first cycle, followed by a fixed dose of 420 mg for the second cycle.

After the completion of the neoadjuvant window cycles, pertuzumab will also be administered as an IV infusion every 21 (±3) days at a fixed dose of 420 mg for an additional six cycles as part of standard-of-care neoadjuvant therapy with TCHP.

For Patients Enrolled in Cohort 2B (Trastuzumab Emtansine and Atezolizumab) of the Neoadjuvant Window Phase:

After completion of the two neoadjuvant window cycles with trastuzumab emtansine, patients in Cohort 2B will receive standard-of-care treatment with TCHP for six cycles every 21 (±3) days. For Cycle 1 of TCHP, pertuzumab will be administered to patients as an IV infusion at a loading dose of 840 mg for the first cycle, followed by a fixed dose of 420 mg for Cycles 2-6 of TCHP.

Administration

Please refer to the pertuzumab Investigator's Brochure for instructions on pertuzumab administration and Table 2 (Safety Evaluation Cohort 1A), Table 4 (Neoadjuvant Window Cohort 2A), and Table 5 (Neoadjuvant Window Cohort 2B).

Trastuzumab Emtansine

The formulation, packaging, and handling should be performed according to the current Trastuzumab Emtansine Investigator's Brochure.

Dose

For Patients Enrolled in Cohort 1B (Trastuzumab Emtansine and Atezolizumab) in the Safety Evaluation Stage:

Trastuzumab emtansine will be administered at a fixed dose of 3.6 mg/kg q3w by IV infusion.

For Patients Enrolled in the Atezolizumab/Trastuzumab Emtansine Safety Expansion Cohort 2C:

Trastuzumab emtansine will be administered at a fixed dose of 3.6 mg/kg q3w by IV infusion.

For Patients Enrolled in Cohort 2B (Trastuzumab Emtansine, Pertuzumab, and Atezolizumab) in the Neoadjuvant Window Cohort Stage:

For patients enrolled in Cohort 2B, trastuzumab emtansine will be administered for two neoadjuvant window cycles as an IV infusion every 21 (±3) days at a fixed dose of 3.6 mg/kg q3w.

Administration

The dose of trastuzumab emtansine will be administered on the basis of the patient's baseline weight. Weight will be measured at each visit. If there is a 10% change in weight compared to baseline the dose should be adjusted accordingly. The dose must be re-adjusted for weight changes 10%.

Administration may be delayed to assess or treat adverse events. Dose reduction will be allowed, following the dose reduction levels described in Example 3 (Assessment of Safety).

Once a dose has been reduced for adverse event(s), it must not be re-escalated. If trastuzumab emtansine is discontinued because of toxicity, it should not be restarted.

Please refer to the Trastuzumab Emtansine Investigator's Brochure for instructions on trastuzumab emtansine administration and Table 3 (Safety Evaluation Cohort 1B and Atezolizumab/Trastuzumab Emtansine Safety Expansion Cohort 2C) and Table 5 (Neoadjuvant Window Cohort 2B).

Dosage and Administration of Chemotherapy Drugs (Neoadjuvant Window Cohorts Only)

Dose

During the neoadjuvant period, carboplatin will be administered as an IV infusion at an initial target of AUC=6 mg/mL×min for patients in Cohort 2A and 2B (TCHP: docetaxel+carboplatin+trastuzumab+pertuzumab) every 21 (±3) days during neoadjuvant treatment for six cycles. Use of anti-emetic medicines is at the discretion of the investigator.

Example 3 (Assessment of Safety) provides guidelines for dosage modification and treatment interruption or discontinuation.

In general, it is recommended to calculate the dose of carboplatin in total milligrams (not milligram per meters squared) using a modified Calvert formula (CrCl is substituted for glomerular filtration rate) as follows:

Total dose (mg)=(target AUC)×(CrCl+25)

CrCl will be either measured or estimated using the Cockroft-Gault formula, as follows:

CrCl (mL/min)=(140−age)×weight (kg)×0.85

72×serum creatinine (mg/dL)

or

CrCl (mL/min)=(140−age)×weight (kg)×0.85

815×serum creatinine (mmol/L)

Calculation of the CrCl will be performed using the baseline weight and the serum creatinine assessed at screening. For subsequent cycles, if no significant change in weight and serum creatinine has occurred according to the investigator's judgment, it is acceptable to maintain the dose of previous cycles.

If there is a 10% change in body weight compared with baseline, the calculation should be revised according to the actual weight. The actual weight becomes the weight of reference for subsequent administrations. Dose should be re-adjusted for weight changes 10%.

Patients for whom this method of calculation is not adequate (e.g., low protein intake, high BSA, low body mass index, etc.), the site may calculate the dose of carboplatin using an appropriate method as per routine practice.

Example 4 (Assessment of Safety) outlines guidelines for dosage modification and treatment interruption or discontinuation.

Administration

Please refer to the carboplatin national prescribing information for instructions on carboplatin administration and Table 4 (Neoadjuvant Window Cohort 2A), and Table 5 (Neoadjuvant Window Cohort 2B).

Docetaxel

Dose

For patients in the neoadjuvant window Cohorts 2A and 2B, docetaxel will be administered as an IV infusion at a dose of 75 mg/m² every 21 (±3) days as part of standard-of-care neoadjuvant TCHP treatment for six cycles.

The dose of docetaxel will be calculated according to the patient's baseline BSA for all cycles. If there is a ≥10% change in body weight compared with baseline, the BSA will be recalculated. Dose should be re-adjusted for weight changes ≥10%. It is not required to cap the total dose of docetaxel for patients with a BSA>2.2 m², but sites are allowed to implement this rule if this is part of their routine practice.

All patients should receive the following premedication prior to each docetaxel dose:

Antiemetic Medication at the Investigator's Discretion

Dexamethasone 8 mg PO, twice a day the day before, the day of, and the day after docetaxel administration. An equivalent dose of other corticosteroids may be substituted (dexamethasone 8 mg=methylprednisone 40 mg=prednisone 50 mg=prednisolone 50 mg).

Administration of parenteral corticosteroids as a substitute for oral dexamethasone prior to neoadjuvant therapy is allowed at the investigator's discretion.

Example 3 outlines guidelines for dosage modification and treatment interruption or discontinuation.

Administration

Please refer to the docetaxel national prescribing information for instructions on docetaxel administration and Table 4 (Neoadjuvant Window Cohort 2A) and Table 5 (Neoadjuvant Window Cohort 2B).

Sequence of Study Drug Administration

All the study drugs are to be administered to patients intravenously. The sequence of administration for the study drugs in each treatment arm should follow the instructions presented in the following Tables 1-5.

TABLE 1
Administration of First and Subsequent Infusions of Atezolizumab

| First Infusion | Subsequent Infusions |
|---|---|
| No premedication is administered. | If patient experienced infusion-related reaction |
| Record patient's vital signs (heart rate, | during any previous infusion, premedication |
| respiratory rate, blood pressure, and | with antihistamines may be administered for |
| temperature) within 60 minutes before starting | Cycles ≥2 at the discretion of the treating |
| infusion. | physician. |
| Infuse (one vial in 250 mL NaCl) over 60 (±15) | Record patient's vital signs (heart rate, |
| minutes. | respiratory rate, blood pressure, and |
| Record patient's vital signs (heart rate, | temperature) within 60 minutes before starting |
| respiratory rate, blood pressure, and | infusion. |
| temperature) during the infusion at 15, 30, 45, | If the patient tolerated the first infusion well |
| and 60 minutes (±5-minute windows are allowed | without infusion-associated adverse events, |
| for all timepoints). | the second infusion may be delivered over |
| Record patient's vital signs (heart rate, | 30 (±10) minutes. |
| respiratory rate, blood pressure, and | If the patient had an infusion-related reaction |
| temperature) at 30 (±10) minutes after the | during the previous infusion, the subsequent |
| infusion. | infusion must be delivered over 60 (±15) |
| Patients will be informed about the possibility of | minutes. |
| delayed post-infusion symptoms and instructed | Record patient's vital signs (heart rate, |
| to contact their study physician if they develop | respiratory rate, blood pressure, and |
| such symptoms. | temperature) during the infusion if clinically |
| | indicated or if the patient experienced |
| | symptoms during the previous infusion. |
| | Record patient's vital signs (heart rate, |
| | respiratory rate, blood pressure, and |
| | temperature) 30 (±10) minutes after the |
| | infusion if clinically indicated or if the patient |
| | experienced symptoms during previous |
| | infusion. |
| | If no reaction occurs, continue subsequent |
| | infusions over 30 (±10) minutes according to |
| | the same schedule for recording vital signs. |

Dose reduction of atezolizumab is not permitted. Guidelines for treatment interruption or discontinuation and the management of specific adverse events are provided in Example 3.

Refer to the Pharmacy Manual for detailed instructions on drug preparation, storage, and administration.

TABLE 2
Treatment Regimen for Metastatic Safety Evaluation Cohort 1A
Atezolizumab/Trastuzumab/Pertuzumab

| | | | | Observation |
|---|---|---|---|---|
| Drug | Dose | Pre-medication | Infusion Period | period |
| Atezolizumab | Loading dose | None | 60 minutes | 30 minutes |
| | 1200 mg | | 30-60 minutes | 30 minutes |
| | Subsequently | | according to | |
| | 1200 mg | | tolerability | |
| Pertuzumab | Loading dose 840 mg | None | 60 minutes | See Pertuzumab |
| | Subsequently 420 mg | | 30-60 minutes | IB |
| | | | according to | |
| | | | tolerability | |
| Trastuzumab | Loading dose | None | 90 minutes | See trastuzumab |
| | 8 mg/kg | | 30-90 minutes | IB |
| | Subsequently | | according to | |
| | 6 mg/kg | | tolerability | |

TABLE 3
Treatment Regimen for Metastatic Safety Evaluation Cohort 1B and
Atezolizumab/Trastuzumab Emtansine Safety Expansion Cohort 2C
Atezolizumab/Trastuzumab Emtansine

| | | | | Observation |
|---|---|---|---|---|
| Drug | Dose | Pre-medication | Infusion Period | period |
| Atezolizumab | Loading dose | None | 60 minutes | 30 minutes |
| | 1200 mg | | 30-60 minutes | 30 minutes |
| | Subsequently | | according to | |
| | 1200 mg | | tolerability | |
| Trastuzumab | 3.6 mg/kg | None | First dose: 90 | 30 minutes if well- |
| emtansine^{a} | | | minutes | tolerated |
| | | | Subsequent | |
| | | | doses: 30-90 | |
| | | | minutes | |
| | | | according to | |
| | | | tolerability | |

^{a}Please refer to Example 3 regarding dose modification guidelines.

TABLE 4
Treatment Regimen for Neoadjuvant Window Cohort 2A

| | | | | |
|---|---|---|---|---|
| | | Pre- | | Observation |
| Drug | Dose | medication | Infusion Period | period |

Atezolizumab/Trastuzumab/Pertuzumab
Neoadjuvant Cycles 1 and 2

| | | | | |
|---|---|---|---|---|
| Atezolizumab | Loading dose 1200 mg | None | 60 minutes | 30 minutes |
| | Subsequently 1200 mg | | 30-60 minutes | 30 minutes |
| | | | according to | |
| | | | tolerability | |
| Pertuzumab | Loading dose 840 mg | None | 60 minutes | See |
| | Subsequently 420 mg | | 30-60 minutes | Pertuzumab |
| | | | according to | IB |
| | | | tolerability | |
| Trastuzumab | Loading dose 8 mg/kg | None | 90 minutes | See |
| | Subsequently 6 mg/kg | | 30-90 minutes | Trastuzumab |
| | | | according to | IB |
| | | | tolerability | |

Standard-of-Care Neoadjuvant TCHP^{a} Cycles 1-6

| | | | | |
|---|---|---|---|---|
| Pertuzumab | 420 mg | None | 30-60 minutes | See |
| | | | according to | Pertuzumab |
| | | | tolerability | IB |
| Trastuzumab | Subsequently 6 mg/kg | None | 30-90 minutes | See |
| | | | according to | Trastuzumab |
| | | | tolerability | IB |
| Docetaxel | 75 mg/m² | Per local | 60 minutes | See |
| Carboplatin | AUC 6 mg/mL × min | standard of | 30-60 minutes | Trastuzumab |
| | | care | | IB |

Surgery
Standard-of-Care Single-Agent Trastuzumab Cycles 7-18

| | | | | |
|---|---|---|---|---|
| Trastuzumab | 6 mg/kg | None | 30-90 minutes | See |
| | | | according to | Trastuzumab |
| | | | tolerability | IB |

AUC = area under the concentration-time curve;
TCH = docetaxel, carboplatin, and trastuzumab.
^{a}Please refer to local prescribing information/institutional guidelines for detailed guidelines on administration and premedications.

TABLE 5
Treatment Regimen for Neoadjuvant Window Cohort 2B

| | | | | |
|---|---|---|---|---|
| | | Pre- | | Observation |
| Drug | Dose | medication | Infusion Period | period |

Atezolizumab/Trastuzumab Emtansine
Neoadjuvant Cycles 1 and 2

| | | | | |
|---|---|---|---|---|
| Atezolizumab | Loading dose 1200 mg | None | 60 minutes | 30 minutes |
| | Subsequently 1200 mg | | 30-60 minutes | 30 minutes |
| | | | according to | |
| | | | tolerability | |
| Trastuzumab | 3.6 mg/kg | None | First dose: 90 | 30 minutes if |
| emtansine | | | minutes | well-tolerated |
| | | | Subsequent doses: | |
| | | | 30-90 minutes | |
| | | | according to | |
| | | | tolerability | |

Standard-of-Care Neoadjuvant TCHP^{a} Cycles 1-6

| | | | | |
|---|---|---|---|---|
| Pertuzumab | Loading dose 840 mg | None | 60 minutes | See |
| | Subsequently 420 mg | | 30-60 minutes | Pertuzumab |
| | | | according to | IB |
| | | | tolerability | |
| Trastuzumab | Loading dose 8 mg/kg | None | 90 minutes | See |
| | Subsequently 6 mg/kg | | 30-90 minutes | Trastuzumab |
| | | | according to | IB |
| | | | tolerability | |
| Docetaxel | 75 mg/m² | Per local | 60 minutes | See national |
| Carboplatin | AUC 6 mg/mL × min | standard of | 30-60 minutes | prescribing |
| | | care | | information |

Surgery
Standard-of-Care Single-Agent Trastuzumab Cycles 7-18

| | | | | |
|---|---|---|---|---|
| Trastuzumab | 6 mg/kg | None | 30-90 minutes | See |
| | | | according to | Trastuzumab |
| | | | tolerability | IB |

AUC = area under the concentration-time curve;
TCH = docetaxel, carboplatin, and trastuzumab.
^{a}Please refer to local prescribing information/institutional guidelines for detailed guidelines on administration and premedications.

### Example 3—Phase Ib Clinical Study: Assessment of Safety

Atezolizumab is not approved and is currently in clinical development. Human experience is currently limited and the entire safety profile is not known at this time. The following information is based on results from nonclinical and clinical studies and published data on similar molecules.

Safety Plan

Measures will be taken to ensure the safety of patients participating in this trial, including the use of stringent inclusion and exclusion criteria and close monitoring. Complete details regarding safety reporting for this study are provided later.

Administration of atezolizumab will be performed in a monitored setting where there is immediate access to trained personnel and adequate equipment and medicines to manage potentially serious reactions. All adverse events and serious adverse events will be recorded during the trial and for up to 30 days after the last dose of study drug or until the initiation of another anti-cancer therapy, whichever occurs first. Investigators are instructed to report all events (adverse events, pregnancy-related adverse events) considered related to study treatment regardless of time after study. The potential safety issues anticipated in this trial, as well as measures intended to avoid or minimize such toxicities, are outlined in the following sections.

Risk Associated with Atezolizumab

The PD-L1/PD-1 pathway is involved in peripheral tolerance; therefore, such therapy may increase the risk of immune-mediated adverse events, specifically the induction or enhancement of autoimmune conditions. Adverse events with potentially immune-mediated causes, including rash, hypothyroidism, hepatitis or transaminitis, colitis, myositis, and myasthenia gravis have been observed in Study PCD4989g. For further details regarding clinical safety, see the atezolizumab Investigator's Brochure.

Although most immune-mediated adverse events observed with immunomodulatory agents have been mild and self-limiting, such events should be recognized early and treated promptly to avoid potential major complications (Di Giacomo et al. The emerging toxicity profiles of anti-CTLA-4 antibodies across clinical indications. Semin Oncol 2010; 37:499-507).

Suggested workup procedures for suspected immune-mediated adverse events are provided in later sections of this example

Risk Associated with Trastuzumab Emtansine

Identified and potential risks of treatment with trastuzumab emtansine are based on all available nonclinical and clinical data relating to trastuzumab emtansine as well as clinical toxicities related to its components (trastuzumab and DM1, a derivative of maytansine), in addition to other DM1-containing ADCs.

Pulmonary toxicity, hepatotoxicity, cardiac toxicity (left ventricular dysfunction), IRR/hypersensitivity, thrombocytopenia (including thrombocytopenia associated with severe hemorrhage), peripheral neuropathy, and extravasation are important identified risks with trastuzumab emtansine and are detailed in the following subsections. Fetal harm and impaired fertility are important potential risks with trastuzumab emtansine.

Please refer to the Trastuzumab Emtansine Investigator's Brochure for a full description of the trastuzumab emtansine safety profile, warnings, precautions, and guidance for investigators.

Pulmonary Toxicity

Cases of interstitial lung disease (ILD), including pneumonitis, some leading to acute respiratory distress syndrome or fatal outcome, have been reported in clinical trials with trastuzumab emtansine. Signs and symptoms include dyspnea, cough, fatigue, and pulmonary infiltrates. These events may or may not occur as sequelae of infusion reactions. Patients with dyspnea at rest as a result of complications of advanced malignancy and/or co-morbidities may be at increased risk of pulmonary events. Treatment has included administration of steroids and oxygen, as well as study drug discontinuation. Upon diagnosis of drug-related ILD or pneumonitis, trastuzumab emtansine treatment must be permanently discontinued.

Hepatotoxicity

Hepatotoxicity, predominantly in the form of asymptomatic increases in the concentrations of serum transaminases (Grades 1-4 transaminitis), has been observed in patients while on treatment with trastuzumab emtansine in clinical trials. Transaminase elevations were generally transient, with peak elevation at Day 8 after therapy administration and subsequent recovery to Grade≤1 value prior to the next cycle. The incidence of increased AST was substantially higher than that for ALT. A cumulative effect of trastuzumab emtansine on transaminases has been observed: the proportion of patients with Grade 1 or 2 increases in elevated transaminases with successive cycles; however, no increase in the proportion of Grade 3 abnormalities over time was observed. The majority of patients with elevated transaminases improved to Grade 1 or the normal range within 30 days of the last dose of trastuzumab emtansine.

Rare cases of severe hepatotoxicity, including death due to DILI and associated hepatic encephalopathy, have been observed in patients treated with trastuzumab emtansine. Some of the observed cases may have been confounded by concomitant medications with known hepatotoxic potential and/or underlying conditions. An acute severe liver injury (Hy's law case) has the following components:

Aminotransferase enzymes are >3×the ULN with concurrent elevation of serum total bilirubin to >2×the ULN (or jaundice), without initial findings of cholestasis (elevated serum alkaline phosphatase).

Cases of NRH of the liver have been identified from liver biopsies in patients treated with trastuzumab emtansine and presenting with signs and symptoms of portal hypertension. NRH confirmed on biopsy was observed leading to fatal hepatic failure. NRH is a rare liver condition characterized by widespread benign transformation of hepatic parenchyma into small regenerative nodules; NRH may lead to non-cirrhotic portal hypertension (Harleb et al. Nodular regenerative hyperplasia: evolving concepts on underdiagnosed cause of portal hypertension. World J Gastroenterol 2011; 17:1400-9). NRH should be considered in patients who develop clinical symptoms of portal hypertension and/or a cirrhosis-like pattern seen on CT scan of the liver but with normal transaminases and no other manifestations of cirrhosis or liver failure following long-term treatment with trastuzumab emtansine. Diagnosis of NRH can only be confirmed by histopathology.

Cardiac Toxicity

Patients treated with trastuzumab emtansine are at risk of developing left ventricular dysfunction. Declines in LVEF<40% have been observed in patients treated with trastuzumab emtansine.

Infusion-Related Reactions/Hypersensitivity

IRRs (anaphylactoid/cytokine release reactions) and hypersensitivity (anaphylactic/allergic reactions) may occur with the administration of monoclonal antibodies and have been reported with trastuzumab emtansine. Treatment with trastuzumab emtansine has not been studied in patients who had trastuzumab permanently discontinued because of an IRR or hypersensitivity reaction; treatment with trastuzumab emtansine is not recommended for these patients.

IRRs that are characterized by one or more of the following symptoms—flushing, chills, pyrexia, dyspnea, hypotension, wheezing, bronchospasm, and tachycardia—have been reported in clinical trials of trastuzumab emtansine. In general, these symptoms were not severe. In most patients, these reactions resolved over the course of several hours to a day after the infusion was terminated. Serious hypersensitivity (anaphylactic-like reactions) has been observed in clinical trials of trastuzumab emtansine.

Administration of trastuzumab emtansine will be performed in a setting with access to emergency facilities and staff who are trained to monitor and respond to medical emergencies. Patients will be observed closely for infusion-related/hypersensitivity reactions during and after each trastuzumab emtansine infusion. Premedication is allowed according to standard practice guidelines. In the event of a true hypersensitivity reaction (in which severity of reaction increases with subsequent infusions), trastuzumab emtansine treatment must be permanently discontinued.

Thrombocytopenia

Thrombocytopenia, or decreased platelet count, was reported in patients in clinical trials of trastuzumab emtansine. The majority of patients had Grade 1 or 2 events (≥50,000/mm³), with the nadir occurring by Day 8 and generally improving to Grade 1 (≥75,000/mm³) by the next scheduled dose. In clinical trials, the incidence and severity of thrombocytopenia were higher in Asian patients. Among Asian patients, the incidence of thrombocytopenia was higher (52.5%) compared with the overall population (30.4%) in Study TDM4370g. However, the incidence rate of Grade 2 hemorrhage did not increase in Asian patients compared with that in the overall population.

Rare cases of bleeding events with a fatal outcome have been observed. Independent of race, cases of severe hemorrhagic events, including CNS hemorrhage, have been reported in clinical trials with trastuzumab emtansine. In some of the observed cases, the patients were also receiving anti-coagulation therapy. Patients on anti-coagulant treatment have to be monitored closely during treatment with trastuzumab emtansine. Platelet counts will need to be monitored prior to each trastuzumab emtansine dose.

Neurotoxicity

Peripheral neuropathy, mainly Grade 1 and predominantly sensory, has been reported in clinical trials of trastuzumab emtansine. Patients should be examined for signs of peripheral neuropathy prior to each dose of trastuzumab emtansine.

Extravasation

In trastuzumab emtansine clinical studies, reactions secondary to extravasation have been observed. The reactions were usually mild and comprised erythema, tenderness, skin irritation, pain, or swelling at the infusion site. Rare reports of more severe events, such as cellulitis, pain (tenderness and burning sensation), and skin irritation, have been received as part of the continuing surveillance of trastuzumab emtansine safety. These reactions have been observed more frequently within 24 hours after infusion. Specific treatment for trastuzumab emtansine extravasation is unknown at this time. The infusion site should be closely monitored for possible subcutaneous infiltration during drug administration.

Pertuzumab

Overall, safety data indicate that pertuzumab is well tolerated and that it can be given in combination with trastuzumab and a range of other therapeutic agents with manageable toxicities. No unexpected toxicities of pertuzumab were encountered other than those known for agents that target the HER family of receptors.

IRRs (chills, fatigue, headache, nausea, and pyrexia), hypersensitivity reactions, anaphylaxis, neutropenia/febrile neutropenia, diarrhea, mucositis, rash, and left ventricular dysfunction are adverse events of particular clinical relevance for this study. Diarrhea has been observed in approximately 60% of patients (treatment-related diarrhea in 50% of patients) being treated with pertuzumab in Phase II single-agent studies and in up to 90% of patients in combination treatment studies. Diarrhea was Grade 1 or 2 in the majority of cases. Rash has been observed in approximately 17% of patients receiving pertuzumab in Phase II single-agent studies and up to 73% of patients in combination studies. The rash was generally of Grade 1 or 2 in severity.

Serious or severe infusion-associated symptoms have been rarely observed in patients receiving pertuzumab. A low rate of cardiac adverse events, predominantly asymptomatic declines in LVEF, has been reported. In the pivotal Phase III Study WO20698/TOC4129g, the rates of symptomatic and asymptomatic LVSD were lower in patients receiving pertuzumab than in those receiving placebo. Because of pertuzumab's role in inhibiting heterodimerization with EGFR, there is a potential risk of ILD with pertuzumab treatment. However, few reports of ILD have been received for patients receiving pertuzumab, and in all cases there were alternative possible causes for the events (e.g., concomitant medication, preceding/concurrent neutropenia with potential infection, relevant medical history). In Study WO20698/TOC4129g, 2.2% of patients receiving pertuzumab developed pneumonitis/ILD, compared with 1.5% of patients receiving placebo. The incidence of Grade 3 adverse events was similar in both treatment arms (0.7% in the pertuzumab arm vs. 0.5% in the placebo arm).

Single-Agent Pertuzumab

The most commonly reported adverse events in patients (n=386) receiving single-agent pertuzumab were diarrhea, fatigue, nausea, vomiting, and decreased appetite. The majority of adverse events reported were Grade 1 or 2 in severity, and the proportion of patients across the pertuzumab program who have discontinued study drug as a result of adverse events is low.

Pertuzumab in Combination with Trastuzumab and Docetaxel

Pertuzumab was well tolerated in combination with trastuzumab (Study WO20697), with an increase in the incidence but not severity of the common adverse events seen with single-agent pertuzumab (notably, diarrhea, rash, and fatigue). Pertuzumab also added little toxicity (predominantly, diarrhea, and febrile neutropenia) to the adverse event profile of trastuzumab and docetaxel when all three drugs were used concurrently (Study WO20698/TOC4129g and Study WO20697) and had minor impact on the doses received, interruptions, discontinuations, or treatment-related mortality. Diarrhea, rash, mucosal inflammation, febrile neutropenia, pruritus, and dry skin were more common (>5% difference) in patients receiving the pertuzumab, trastuzumab, and docetaxel regimen than in patients in the placebo arm in Study WO20698/TOC4129g.

Importantly, despite targeting the same HER2 pathway, pertuzumab adds no significant cardiac toxicity when given with trastuzumab (with or without chemotherapy).

An increased incidence of febrile neutropenia was observed for Asian patients in both treatment arms compared with patients of other races and from other geographic regions. Among Asian patients, the incidence of febrile neutropenia was higher in the pertuzumab group (26%) compared with that in the placebo group (12%) in Study WO20698/TOC4129g.

Please refer to the Pertuzumab Investigator's Brochure for full description of the pertuzumab safety profile, warnings, precautions, and guidance for investigators.

For pertuzumab, allergic infusion-associated reactions (chills, diarrhea, fatigue, headache, nausea, pyrexia and especially hypersensitivity reactions), respiratory events (ILD) and cardiac dysfunction are adverse events of particular clinical relevance.

Docetaxel, Carbolatin, and Trastuzumab

For adverse reactions, warnings, and precautions for docetaxel and carboplatin, refer to national prescribing information. Respective information regarding trastuzumab can be found in the Trastuzumab Investigator's Brochure.

General Plan to Manage Safety Concerns

Safety will be evaluated in this study through the monitoring of all serious and non-serious adverse events defined and graded according to NCI CTCAE v4.0.

General safety assessments will include serial interval histories, physical examinations, and specific laboratory studies, including serum chemistry and blood counts.

During the study, patients will be closely monitored for the development of any signs or symptoms of autoimmune conditions and infection.

All serious adverse events and protocol-defined events of special interest will be reported in an expedited fashion.

Dose Modifications

Reasons for dose modifications or delays, the supportive measures taken, and the outcomes will be documented in the patient's chart and recorded on the eCRF. The severity of adverse events will be graded according to the NCI CTCAE v4.0.

Dose reduction of atezolizumab is not permitted.

For any concomitant conditions reported at baseline, the dose modifications will apply according to the corresponding shift in toxicity grade, if the investigator determines that it is appropriate. For example, if a patient has Grade 1 asthenia at baseline that increases to Grade 2 during treatment, this will be considered a shift of one grade and treated as Grade 1 toxicity for dose-modification purposes.

When several toxicities with different grades of severity occur at the same time, the dose modifications should be according to the highest grade observed.

If, in the opinion of the investigator, a toxicity is considered to be attributable solely to one component of the study treatment (i.e., atezolizumab, trastuzumab, trastuzumab emtansine, pertuzumab) and the dose of that component is delayed or modified in accordance with the guidelines below, the other component may be administered if there is no contraindication.

Note that pertuzumab should not be given without trastuzumab, and pertuzumab should be discontinued if trastuzumab is not given.

When study treatment is temporarily interrupted because of toxicity caused by atezolizumab, trastuzumab, and/or pertuzumab, the treatment cycles will be restarted such that the atezolizumab/trastuzumab/pertuzumab or atezolizumab/trastuzumab emtansine infusions remain synchronized.

The treating physician may use discretion in modifying or accelerating the dose modification guidelines described below, depending on the severity of toxicity and an assessment of the risk versus benefit for the patient, with the goal of maximizing patient compliance and access to supportive care.

Dose Modification for Atezolizumab

There will be no dose reduction for atezolizumab in this study. Patients may temporarily suspend study treatment if they experience toxicity that is considered related to study drug (atezolizumab) and requires a dose to be held. If atezolizumab is held because of related adverse events for >42 days beyond when the next dose would have been given, then the patient will be discontinued from atezolizumab treatment and will be followed for safety and efficacy as specified in Example 4. If, in the judgment of the investigator, the patient is likely to derive clinical benefit from resuming atezolizumab after a hold >42 days, study drug may be restarted with the approval of the Medical Monitor.

If patients must be tapered off steroids for the treatment of adverse events, study treatment may be held for >42 days until steroids are discontinued or reduced to prednisone dose (or dose equivalent)≤10 mg/day. The acceptable length of interruption will depend on agreement between the investigator and the Medical Monitor.

Dose interruptions for reason(s) other than adverse events, such as surgical procedures, may be allowed with Medical Monitor approval. The acceptable length of interruption will depend on agreement between the investigator and the Medical Monitor.

Management of Atezolizumab-Specific Adverse Events

Toxicities associated or possibly associated with atezolizumab treatment should be managed according to standard medical practice. Additional tests, such as autoimmune serology or biopsies, should be used to determine a possible immunogenic etiology.

Discontinuation of atezolizumab may not have an immediate therapeutic effect and, in severe cases, immune-mediated toxicities may require acute management with topical corticosteroids, systemic corticosteroids, mycophenolate mofetil, or TNF-a inhibitors.

The investigator should consider the benefit-risk balance that a patient may be experiencing prior to further administration of atezolizumab. Atezolizumab should be permanently discontinued in patients with life-threatening, immune-mediated adverse events. The following sections provide guidance for handling specific adverse events.

Gastrointestical Toxicity

Immune-mediated colitis has been associated with the administration of atezolizumab.

The guidelines on how to manage gastrointestinal toxicity for patients treated with atezolizumab are located in Table 6.

TABLE 6
Guidelines for Managing Atezolizumab-Associated Gastrointestinal
Toxicity

| Diarrhea | Management |
|---|---|
| Grade 2 | Hold atezolizumab and discontinue NSAIDs (or other |
| (4-6 stools/day over | medications that exacerbate colitis). |
| baseline) <5 days | Investigate for etiology. |
| | Restart atezolizumab once at baseline stool frequency. |
| Grade 2 | Hold atezolizumab and discontinue NSAIDs (or other |
| (4-6 stools/day over | medications that exacerbate colitis) while the etiology is |
| baseline) >5 days | being investigated. |
| | Consider referral to a gastroenterologist. |
| | Administer anti-diarrheal agent (e.g., loperamide). |
| | Consider oral budesonide, mesalamine, or 10 mg/day of |
| | oral prednisone or equivalent. |
| | Restart atezolizumab once at baseline stool frequency. |
| Abdominal pain | Hold atezolizumab and discontinue NSAIDs (or other |
| Blood or mucus in | medications that exacerbate colitis). |
| stool | Rule out bowel perforation. Consider administering |
| or | 60 mg/day of prednisone or equivalent (oral budesonide or IV |
| | methylprednisolone). |
| Grade ≥3 | Taper steroids over 1 month. Restart atezolizumab if |
| ≥7 stools/day over | diarrhea is resolved, as confirmed by sigmoidoscopy or |
| baseline) with | colonoscopy, and systemic steroid dose is ≤10 mg/day of |
| peritoneal signs, | oral prednisone or equivalent. |
| ileus, or fever | Consider TNF antagonists for refractory diarrhea. |
| | Permanently discontinue atezolizumab for life-threatening |
| | immune-mediated diarrhea or colitis. |

IV = intravenous;
NSAID = non-steroidal anti-inflammatory drug;
TNF = tumor necrosis factor.

If the event is of significant duration (>5 days) or is associated with signs of systemic inflammation or acute-phase reactants (e.g., increased C-reactive protein, platelet count, or bandemia), it is recommended to do the following:
Perform sigmoidoscopy (or colonoscopy, if appropriate) with colonic biopsy, with three to five specimens for standard paraffin block to check for inflammation and lymphocytic infiltrates in order to confirm colitis diagnosis.
Perform laboratory tests to rule out alternate etiology (i.e., WBCs and stool calprotectin).

Hepatotoxicity

Immune-mediated hepatitis has been associated with the administration of atezolizumab. Eligible patients must have adequate liver function, as manifested by measurements of total bilirubin and hepatic transaminase, and liver function will be monitored throughout study treatment.

During this study, patients presenting with right upper-quadrant abdominal pain and/or unexplained nausea or vomiting should have LFTs performed immediately and reviewed before administration of the next dose of study drug.

If LFTs increase, neoplastic, concurrent medications, viral hepatitis, and toxic etiologies should be considered and addressed, as appropriate. Imaging of the liver, gall bladder, and biliary tree should be performed to rule out neoplastic or other causes for the increased LFTs. Anti-nuclear antibody, perinuclear anti-neutrophil cytoplasmic antibody, anti-liver kidney microsomal antibodies, and anti-smooth muscle antibody tests should be performed if an autoimmune etiology is considered.

Patients with LFT abnormalities should be managed according to the guidelines in Table 7.

TABLE 7
Guidelines for Managing Atezolizumab-Associated Hepatotoxicity

| LFT | |
|---|---|
| Abnormalities | Management |
| Grade 1 events | Continue therapy |
| | Continue LFT monitoring |
| Grade 2 events | Monitor LFTs more frequently until return to baseline |
| | values |
| | If persists >5-7 days: hold therapy and start 60 mg |
| | prednisone or equivalent per day; when LFTs ≤G1, |
| | taper steroids over ≥1 month, resume therapy when |
| | systemic steroid dose is ≤10 mg oral prednisone |
| | equivalent per day |
| Grade 3-4 | Discontinue therapy |
| events | Consider GI consult and liver biopsy to establish |
| | etiology of hepatic injury if necessary |
| | Start 60 mg prednisone or equivalent per day |
| | If LFT results do not decrease within 48 h after initiation |
| | of systemic steroids, addition of an alternative |
| | immunosuppressive agent (e.g., mycophenolate or |
| | TNF-α antagonist) may be considered. |
| | Taper steroids over ≥1 month, when symptoms improve |
| | to G0 or G1 |
| | *Contact medical monitor if atezolizumab is |
| | discontinued. |

GI = gastronintestinal;
LFT = liver function test;
TNF-α = tumor necrosis factor-alpha.

Dermatologic Toxicity

Treatment-emergent rash has been associated with atezolizumab. The majority of the cases of rash were mild in severity and self-limited, with or without pruritus.

A dermatologist should evaluate persistent and/or severe rash or pruritus. A biopsy should be considered unless contraindicated.

Dermatologic toxicity and rash should be managed according to the guidelines in Table 8.

TABLE 8
Guidelines for Managing Atezolizumab-Associated
Dermatologic Toxicity

| Dermatologic | |
|---|---|
| Toxicity/Rash | |
| (e.g., Maculopapular | |
| or Purpura) | Management |
| Grade 1, mild, <10% | Continue atezolizumab; symptomatic therapy |
| BSA | with antihistamine PRN. |
| | Consider topical steroids and/or other |
| | symptomatic therapy (e.g., antihistamines). |
| Grade 2, moderate, | Continue atezolizumab. |
| 10%-30% BSA | Consider dermatologist referral. |
| | Administer topical steroids. |
| | Consider higher potency topical steroids if rash |
| | unresolved. |
| Grade 3, severe, | Hold atezolizumab. |
| >30% BSA | Consult dermatologist. |
| | Administer 10 mg/day of oral prednisone or |
| | equivalent. If rash is unresolved after 48-72 |
| | hours, administer 60 mg of oral prednisone or |
| | equivalent. |
| | Restart atezolizumab if rash resolved and |
| | systemic dose is ≤10 mg/day of oral prednisone |
| | or equivalent. |
| | Permanently discontinue atezolizumab for |
| | life-threatening immune-mediated dermatologic |
| | toxicity. |

BSA = body surface area;
PRN = as needed.

Endocrine Toxicity

Hypothyroidism has been associated with the administration of atezolizumab.

Patients with unexplained symptoms, such as fatigue, myalgias, impotence, changes in mental status, or constipation should be investigated for the presence of thyroid, pituitary, or adrenal endocrinopathies, as well as for hyponatremia or hyperkalemia. An endocrinologist should be consulted if an endocrinopathy is suspected. TSH and free T4 levels should be obtained to determine if thyroid abnormalities are present. TSH, prolactin, and a morning cortisol level will help to differentiate primary adrenal insufficiency from primary pituitary insufficiency.

Hypothyroidism should be managed according to the guidelines in Table 9.

TABLE 9
Guidelines for Managing Atezolizumab-Associated
Endocrine Toxicity

| Hypothyroidism | Management |
|---|---|
| TSH elevated | Continue atezolizumab. |
| asymptomatic | Start thyroid replacement hormone. |
| | Monitor TSH weekly. |
| TSH elevated | Hold atezolizumab. |
| symptomatic | Consider referral to an endocrinologist. |
| | Restart atezolizumab when symptoms are controlled |
| | by thyroid replacement and TSH levels are |
| | decreasing. |

TSH = thyroid-stimulating hormone.

Patients with persistent hyperglycemia should be evaluated for the potential of immune-mediated pancreatic endocrine insufficiency with measurement of serum C-peptide and islet-cell or anti-glutamic acid decarboxylase auto-antibodies. Consultation with an endocrinologist is appropriate in this setting.

Pulmonary Toxicity

Dyspnea, cough, fatigue, hypoxia, and pulmonary infiltrates have been associated with the administration of atezolizumab and have primarily been observed in patients with underlying NSCLC.

Appropriate workup for pulmonary adverse events should include the following as appropriate, as well as ruling out alternative causes (e.g., lymphangitic carcinomatosis, infection, heart failure, chronic obstructive pulmonary disease, or pulmonary hypertension):
Measurement of oxygen saturation (i.e., arterial blood gas)
High-resolution CT scan of the chest
Bronchoscopy with bronchoalveolar lavage and biopsy (if clinically feasible)
Pulmonary function tests (diffusion capacity of the lung for carbon monoxide [DL_{co}])

Pulmonary function testing and CT with a pulmonary embolism protocol may also be helpful in the diagnostic evaluation. For patients with clinical symptoms, treatment should include administration of corticosteroids and/or oxygen when indicated. Consultation with a pulmonologist is appropriate for a suspected lung immune-mediated adverse event, and a bronchoscopy with biopsy should be performed, unless contraindicated, prior to the administration of corticosteroids.

Patients will be assessed for pulmonary signs and symptoms throughout the study.

Pulmonary toxicity should be managed according to the guidelines in Table 10.

TABLE 10
Guidelines for Managing Atezolizumab-Associated
Pulmonary Toxicity

| Pulmonary | |
|---|---|
| Toxicity | Management |
| Grade 1 | Permanently discontinue study treatment with close |
| | monitoring |
| | Re-evaluate on serial imaging |
| | Consider pulmonary consultation |
| | *Contact medical monitor if atezolizumab is discontinued. |
| Grade 2 | Permanently discontinue study treatment |
| | Pulmonary and infectious diseases consultation with |
| | consideration for bronchoscopy/bronchoalveolar lavage |
| | Start 60 mg prednisone or equivalent per day |
| | When improves to G0 or G1, then taper steroids over |
| | ≥1 month |
| | Treat as G3/4 if recurrent episode of pneumonitis |
| | *Contact medical monitor if atezolizumab is discontinued. |
| Grade 3 and | Permanently discontinue study treatment |
| 4 events | Bronchoscopy/bronchoalveolar lavage is recommended |
| | Start 60 mg prednisone or equivalent per day. |
| | Taper steroids over ≥1 month once symptoms improve to |
| | G0 or 1. |
| | If not improving after 48 h or worsening: add additional |
| | immunosuppression (e.g., infliximab, cyclophosphamide, |
| | Intravenous immunoglobulin, or mycophenolate mofetil) |
| | *Contact medical monitor if atezolizumab is discontinued. |

Potential Pancreatic Toxicity

Symptoms of abdominal pain associated with elevations of amylase and lipase, suggestive of pancreatitis, have been associated with the administration of other immunomodulatory agents. The differential diagnosis of acute abdominal pain should include pancreatitis. Appropriate workup should include an evaluation for obstruction, as well as serum amylase and lipase tests.

Pancreatic toxicity should be managed according to the guidelines in Table 11.

TABLE 11
Guidelines for Managing Atezolizumab-Associated
Pancreatic Toxicity

| Amylase/Lipase | |
|---|---|
| Abnormalities | Management |
| Amylase/lipase | Continue atezolizumab. |
| (greater than the ULN and | Monitor amylase/lipase levels prior to dosing. |
| ≤2 × ULN) and patient is | |
| asymptomatic | |
| Amylase/lipase | Continue atezolizumab. |
| (>2 × ULN to ≤5 × ULN) and | Monitor amylase/lipase weekly. |
| patient is asymptomatic | Consider 10 mg/day of oral prednisone or equivalent |
| | for prolonged elevation (e.g., more than 3 weeks). |
| Amylase/lipase (>5 × ULN) | Hold atezolizumab. |
| and patient is asymptomatic | Monitor amylase/lipase every other day and consider |
| | 60 mg/day of oral prednisone or equivalent. |
| | Consult a gastroenterologist. |
| | Resume atezolizumab dosing when lipase/amylase |
| | levels are <2 × ULN. |
| Autoimmune pancreatitis | Hold atezolizumab. |
| (abdominal pain and raised | Consult a gastroenterologist. |
| amylase/lipase levels) | Consider administering IV steroids (60 mg/day of |
| | prednisone or equivalent) followed by taper over |
| | 2-4 weeks. |
| | Resume atezolizumab when enzyme levels are |
| | <2 × ULN and patient is asymptomatic. |
| | Permanently discontinue atezolizumab for |
| | life-threatening immune-mediated pancreatitis. |

IV = intravenous;
ULN = upper limit of normal.

Potential Eye Toxicity

Patients in the study are encouraged to maintain eye hydration, generally through the use of moisturizing eye drops.

An ophthalmologist should evaluate visual complaints. Uveitis or episcleritis may be treated with topical corticosteroid eye drops. Atezolizumab should be permanently discontinued for immune-mediated ocular disease that is unresponsive to local immunosuppressive therapy.

Ocular toxicity should be managed according to the guidelines in Table 12.

TABLE 12
Guidelines for Managing Atezolizumab-Associated Eye Toxicity

| Description | Management |
|---|---|
| Symptomatic eye toxicity | Hold atezolizumab. |
| (autoimmune uveitis, iritis | Consult ophthalmologist and start topical |
| or episcleritis) | corticosteroid eye drops. |
| | Atezolizumab may be restarted following |
| | resolution of the events and discussions |
| | with the Medical Monitor. |
| | Permanently discontinue atezolizumab for |
| | immune-mediated ocular disease that is |
| | unresponsive to local immunosuppressive |
| | therapy. |

Systemic Immune Activation

SIA is a rare condition characterized by an excessive immune response. Given the mechanism of action of atezolizumab, SIA is considered a potential risk. SIA should be included in the differential diagnosis for patients who develop a sepsis-like syndrome after administration of atezolizumab, and initial workup should include serum ferritin, complete blood count, liver function tests, serum triglycerides, and a coagulation profile. In the event of suspected SIA, the Medical Monitor should be contacted for additional recommendations. Treatment with agents such as tocilizumab, as well as corticosteroids, should be considered in the event of SIA.

Study Drug Modification and Management of Specific Adverse Events

IRRs are known to occur with the administration of monoclonal antibodies and have been reported with trastuzumab, trastuzumab emtansine, and pertuzumab. IRRs and hypersensitivity reactions should be managed according to the guidelines in Table 13.

TABLE 13
Guidelines for the Management of Infusion-Related Reactions to HER2-
Directed Therapy (Trastuzumab, Pertuzumab, and Trastuzumab Emtansine)

| Event | Action to Be Taken with HER2-Directed Therapy |
|---|---|
| Grade 4 IRR, allergic, or | Stop infusion. Study treatment should be permanently |
| hypersensitivity reaction | discontinued |
| | Supportive care with oxygen, β-agonists, antihistamines, |
| | antipyretics, or corticosteroids may be used, as appropriate, at |
| | the investigator's discretion. |
| | Patients should be monitored until complete resolution of |
| | symptoms. |
| | Supportive care with oxygen, β-agonists, antihistamines, |
| | antipyretics, or corticosteroids may be used, as appropriate, at |
| | the investigator's discretion. Patients should be monitored until |
| | complete resolution of symptoms. |
| Grade 3 IRR, allergic, or | May re-treat at investigator's discretion. In the event of a true |
| hypersensitivity reaction | hypersensitivity reaction (in which the severity of reaction |
| | increases with subsequent infusions) study treatment should be |
| | permanently discontinued. |
| | Premedication with corticosteroids, antihistamines, and |
| | antipyretics may be used before subsequent infusions at the |
| | investigator's discretion. |
| | Decrease infusion rate by 50% or interrupt infusion. Supportive |
| | care with oxygen, β-agonists, antihistamines, antipyretics, or |
| | corticosteroids may be used as appropriate at the investigator's |
| | discretion. Patients should be monitored until complete |
| | resolution of symptoms. |

IRR = infusion-related reaction.

Guidelines for dose modification or delays because of hepatotoxicity, thrombocytopenia, ILD or pneumonitis, and neuropathy adverse events for patients in Cohort 2B treated with trastuzumab emtansine are presented in Table 14.

TABLE 14
Dose Modifications or Delays for Trastuzumab Emtansine

| Event | Action to Be Taken |
|---|---|
| Hepatotoxicity | |
| ALT | For a Grade 2 or 3 increase in ALT that occurs on |
| | the laboratory evaluation for cycle Day 1 or the |
| | planned day of dosing, withhold trastuzumab |
| | emtansine until ALT recovers to Grade ≤1. Resume |
| | with dose reduction by one level for Grade 2 or 3 |
| | elevations. |
| | For a Grade 4 increase in ALT, discontinue |
| | trastuzumab emtansine. A repeat laboratory |
| | evaluation (within 24 hours) may be done to exclude |
| | laboratory error prior to discontinuing study |
| | treatment. |
| AST | For a Grade 2 increase in AST on the laboratory |
| | evaluation for cycle Day 1 or the planned day of |
| | dosing, withhold trastuzumab emtansine until AST |
| | recovers to Grade ≤1. Resume without dose |
| | reduction when AST has recovered |
| | For Grade 3 increase in AST on the laboratory |
| | evaluation for cycle Day 1 or the planned day of |
| | dosing, withhold trastuzumab emtansine until AST |
| | recovers to Grade ≤1. Resume with dose reduction |
| | by one level when AST has recovered. |
| | For Grade 4 increase in AST, discontinue |
| | trastuzumab emtansine. A repeat laboratory |
| | evaluation (within 24 hours) may be done to exclude |
| | laboratory error prior to discontinuing study |
| | treatment. |
| Total bilirubin (TBILI) | For TBILI ≥1.0 × ULN to ≤2.0 × ULN that occurs on |
| | Day 1 laboratory evaluation of each cycle or the day |
| | of planned dosing, withhold trastuzumab emtansine |
| | until TBILI recovers to ≤1.0 × ULN (or direct bilirubin |
| | recovers to ≤1.0 × ULN for patients with Gilbert |
| | syndrome). For TBILI elevations >1.0 × ULN |
| | to ≤2.0 × ULN, resume when recovered, with a one- |
| | level dose reduction. |
| | For TBILI >2 × ULN at any time (or direct |
| | bilirubin >2 × ULN for patients with Gilbert |
| | syndrome), discontinue trastuzumab emtansine and |
| | report the event as a serious adverse event (if |
| | applicable) or non-serious expedited adverse event |
| | (if applicable). |
| | Note: Assess AST, ALT, and TBILI weekly or as |
| | medically indicated until recovery. Allow a maximum |
| | dose delay of 42 days from the last administered |
| | dose to recovery as described above or otherwise |
| | discontinue study treatment. |
| | For ALT or AST >3.0 × ULN concurrent with |
| | TBILI >2.0 × ULN, permanently discontinue |
| | trastuzumab emtansine. |
| Nodular regenerative hyperplasia (NRH) | For any clinical signs of liver dysfunction, discontinue |
| | trastuzumab emtansine and have the patient |
| | evaluated by a hepatologist. If there are signs of |
| | portal hypertension (e.g., ascites and/or varices) and |
| | a cirrhosis-like pattern is seen on CT scan of the |
| | liver, the possibility of NRH should be considered. |
| | Liver biopsy may confirm but is not necessary for |
| | diagnosis. Trastuzumab emtansine should be |
| | permanently discontinued in the event of a diagnosis |
| | of NRH. |
| Thrombocytopenia | |
| Grade 2 or 3 on day of scheduled | Assess platelet counts weekly or as medically |
| treatment | indicated until recovery. Withhold study treatment |
| | until Grade ≤1. Resume treatment without dose |
| | reduction. If a patient requires two delays due to |
| | thrombocytopenia, consider reducing dose by one |
| | level. |
| Grade 4 at any time | Assess platelet counts weekly or as medically |
| | indicated until recovery. Withhold trastuzumab |
| | emtansine until Grade ≤1, then resume with one- |
| | level dose reduction (i.e., from 3.6 to 3 mg/kg or from |
| | 3 to 2.4 mg/kg) in subsequent cycles. If event |
| | occurs at 2.4-mg/kg dose, discontinue study |
| | treatment. |
| Other hematology toxicity | |
| Grade ≥3 | Withhold study treatment until recovery to Grade ≤2. |
| | Weekly CBC assessments should be done until |
| | recovery or as medically indicated. |
| | A maximum dose delay of 42 days from the last |
| | administered dose to Grade ≤2 or baseline value will |
| | be allowed; otherwise, patient must be discontinued |
| | from study treatment. |
| Interstitial lung disease or pneumonitis | |
| Grade 1 or 2 | Permanently discontinue study treatment if not |
| | radiotherapy related. For symptomatic (Grade 2) |
| | radiotherapy-related pneumonitis, discontinue if not |
| | resolving with standard treatment (e.g., steroids). |
| | Relationship to radiotherapy should be determined |
| | on the basis of timing and location of radiographic |
| | abnormalities relative to the radiotherapy. |
| | Patients discontinued from trastuzumab emtansine |
| | for pneumonitis may not continue study treatment |
| | with trastuzumab. |
| Grade 3 or 4 | Permanently discontinue all study treatment |
| | regardless of attribution. |
| Neuropathy | |
| Grade 1 paresthesias or dysesthesias | Maintain trastuzumab emtansine dose. |
| that do not interfere with function | |
| Grade 2 paresthesias or dysesthesias | Maintain trastuzumab emtansine dose. |
| interfering with function, but not | |
| activities of daily living | |
| Grade 3 paresthesias or dysesthesias | Withhold therapy dose until neuropathy Grade <3. |
| with pain or with function impairment | May consider reducing one dose level. |
| interfering with activities of daily living | |
| Grade 4 persistent paresthesias or | Discontinue therapy if event does not resolve to |
| dysesthesias that are disabling or life | Grade ≤3 within 42 days. |
| threatening | |
| Other clinically significant adverse events | |
| Grade 3 or 4 | Decrease one dose level if clinically significant. |

CT = computed tomography; NRH = nodular regenerative hyperplasia; TBILI = total bilirubin; ULN = upper limit of normal.

Guidelines for dose modification or delays in patients because of adverse events for patients in Cohort 2B receiving pre-operative treatment with TCHP are presented in Table 15.

TABLE 15
Dose Modifications and Delays for Pre-Operative TCHP

| | Carboplatin and Docetaxel Dose Modification ^{a} | | |
|---|---|---|---|
| | | | Adverse Events That |
| | Intra-Cycle Adverse Events That | | Cause a Delay in |
| NCI CTCAE v4.0 Event | Resolve prior to Day 21 | | Treatment Cycle |
| Neutrophils, Grades 2 | First event | Maintain dose | Do not administer until |
| (1000-1199 cells/mm³), 3 | | | ANC ≥1200 cells/mm³. |
| and 4 | | | |
| | | | If recovery takes 1-3 |
| | | | weeks, maintain dose; add |
| | | | G-CSF to subsequent |
| | | | cycles. |
| | Subsequent | Maintain dose. | Do not administer until |
| | events (patient | | ANC is ≥1200 cells/mm³. |
| | receiving G-CSF) | | |
| | | | Decrease carboplatin one |
| | | | dose level and continue |
| | | | G-CSF support. |
| | | | If second episode with |
| | | | G-CSF support and |
| | | | previous carboplatin |
| | | | reduction, decrease |
| | | | docetaxel one dose level. |
| Platelets, Grades 2 and 3 | First event | Maintain dose. | Do not administer |
| | | | until ≥100,000 |
| | | | cells/mm³; |
| | | | decrease carboplatin one |
| | | | dose level. |
| | Second event | Maintain dose. | Do not administer |
| | | | until ≥100,000 |
| | | | cells/mm³; |
| | | | decrease carboplatin |
| | | | additional dose level. |
| | Third event | Maintain dose. | Do not administer |
| | | | until ≥100,000 |
| | | | cells/mm³; |
| | | | decrease docetaxel one |
| | | | dose level; maintain |
| | | | carboplatin dose or |
| | | | discontinue carboplatin. |
| Platelets, Grade 4 | | Decrease | Do not administer |
| | | carboplatin one | until ≥100,000 |
| | | dose level. | cells/mm³; |
| | | | decrease carboplatin one |
| | | | dose level. |
| Hepatic function | | | |
| Hepatic bilirubin or | Maintain dose. | | Do not administer until |
| AST/ALT or Grade 2 | | | hepatic abnormality |
| alkaline phosphatase | | | returns to Grade ≤1 and |
| | | | maintain dose level. |
| Grade 3 | Maintain dose. | | Do not administer until |
| | | | hepatic abnormality |
| | | | returns to Grade ≤1 and |
| | | | reduce docetaxel one dose |
| | | | level. |
| Grade 4 | If patient has recovered by Day 21, | | Discontinue. |
| | reduce docetaxel and carboplatin by | | |
| | one dose level or discontinue. | | |
| Renal function | | | |
| Renal serum creatinine, | | | Do not administer until |
| Grades 2 and 3 | | | serum creatinine is |
| | | | Grade <1 and calculated |
| | | | CrCl is ≥30 mL/min. |
| | | | If CrCl is >50 mL/min, |
| | | | maintain dose. If CrCl is |
| | | | 30-50 mL/min, decrease |
| | | | carboplatin one dose level. |
| | | | However, if calculated |
| | | | CrCl is <30 mL/min and all |
| | | | other non-renal function |
| | | | adverse events have |
| | | | resolved to Grade <1 on |
| | | | scheduled Day 1, |
| | | | carboplatin should be |
| | | | discontinued and |
| | | | docetaxel may be |
| | | | administered. |
| Grade 4 | Discontinue. | | Discontinue. |
| Neuropathy | | | |
| Grade 1 paresthesias or | Maintain dose of both agents. | | Maintain dose of both |
| dysesthesias that do not | | | agents. |
| interfere with function | | | |
| Grade 2 paresthesias or | Maintain dose of both agents. | | Decrease carboplatin one |
| dysesthesias interfering | | | dose level. Maintain |
| with function, but not | | | docetaxel dose. |
| activities of daily living | | | |
| Grade 3 paresthesias or | Maintain dose of both agents. | | Do not administer therapy |
| dysesthesias with pain or | | | until neuropathy Grade <3. |
| with function impairment | | | Decrease docetaxel and |
| interfering with activities | | | carboplatin one dose level. |
| of daily living | | | |
| Grade 4 persistent | Discontinue therapy. | | Discontinue therapy. |
| paresthesias or | | | |
| dysesthesias that are | | | |
| disabling or life | | | |
| threatening | | | |
| Other clinically significant | All events | Decrease one | Decrease one dose level if |
| adverse events | | dose level if | clinically appropriate. |
| | | clinically | |
| | | appropriate. | |

ANC = absolute neutrophil count; CrCl = creatinine clearance; G-CSF = granulocyte colony stimulating factor; NCI CTCAE = National Cancer Institute Common Terminology Criteria for Adverse Events; TCHP = docetaxel, carboplatin, trastuzumab, and pertuzumab.
^{a} Dose modifications apply to both drugs unless otherwise specified.

Safety Cardiovascular Assessments and Dose Modifications for Cardiovascular Events

All patients will undergo scheduled LVEF (Left Ventricular Ejection Fraction) assessments on ECHO or MUGA (Multiple Gated Acquisition) scans. The results of the LVEF assessments will be used to determine whether trastuzumab, pertuzumab, and trastuzumab emtansine administration may be continued.

Asymptomatic Decrease in LVEF

Patients without significant cardiac history and with a baseline LVEF 50% as determined on ECHO or MUGA scan are eligible for study participation. Ejection fractions will be monitored during the last week of Cycle 2 and every four cycles thereafter (at any timepoint within respective cycles) until assessment at the study treatment discontinuation or early termination visit.

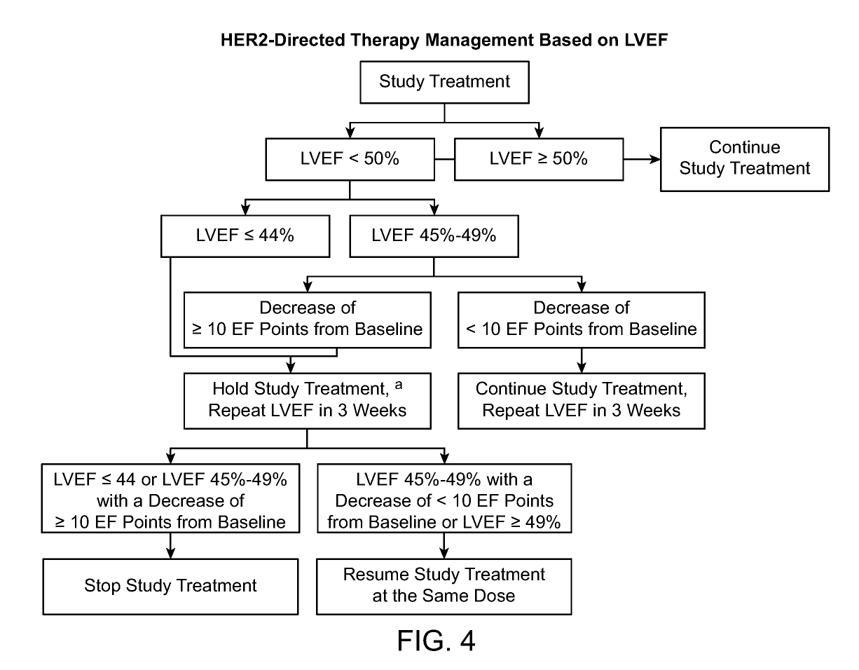

The management of patients treated with trastuzumab and pertuzumab or trastuzumab emtansine based on measured LVEF and changes in LVEF from baseline in patients is presented in FIG. 4. If the LVEF is reported as a range, the median of the range should be used. If an investigator is concerned that an adverse event may be related to cardiac dysfunction, an additional LVEF measurement may be performed. Trastuzumab and pertuzumab or trastuzumab emtansine will be discontinued in any patient who develops symptomatic CHF. CHF should be treated and monitored according to standard medical practice.

The decision to stop or continue trastuzumab and pertuzumab or trastuzumab emtansine should be based on the algorithm shown in FIG. 4. Treatment with trastuzumab and pertuzumab, or trastuzumab emtansine must be discontinued in all patients for whom a confirmed decrease to ≤44% in LVEF is documented (with a repeat assessment within 21 days). Treatment with trastuzumab and pertuzumab or trastuzumab emtansine should be held for patients whose LVEF decreases to values of 45%-49%, with an absolute decrease of ≥10-percentage points in LVEF from baseline. For these patients, assessment of LVEF should be repeated within 21 days and treatment with trastuzumab and pertuzumab or trastuzumab emtansine should be discontinued if the LVEF has not recovered to within 10% absolute difference below baseline. If clinically significant cardiac dysfunction or cardiac failure develops or persists, or if significant medical management is required to maintain the ejection fraction, the patient should be discontinued from study treatment.

Patients should be permanently discontinued from HER2-targeted treatment Grade 3 or 4 LVSD, Grade 3 or 4 heart failure, or Grade 2 heart failure accompanied by a decline <45% in LVEF. A serious adverse event should be reported.

### Example 4—Phase II Clinical Study: Objectives and Endpoints, Study Design

Objectives and Endpoints

This Phase II, Double-Blind, Randomized, Placebo-Controlled Multicenter Study will evaluate the efficacy and safety of trastuzumab emtansine in combination with atezolizumab or atezolizumab-placebo in patients with HER2-positive, locally advanced or MBC, who have receved prior trastuzumab and taxane based therapy, either alone or in combination, and/or who have progressed within 6 months after completing adjuvant therapy.

Efficacy Objectives

Primary Efficacy Objective

The primary efficacy objective for this study is to evaluate the efficacy of the combination of trastuzumab emtansine plus atezolizumab compared with trastuzumab emtansine plus placebo on the basis of the following endpoint:
Progression-free survival (PFS), defined as the time from randomization to the first occurrence of disease progression, as determined by investigator assessment using RECIST v1.1, or death from any cause, whichever occurs first.

Secondary Efficacy Objectives

The secondary efficacy objectives for this study are to evaluate the efficacy of the combination of trastuzumab emtansine plus atezolizumab compared with trastuzumab emtansine plus placebo on the basis of the following endpoints:
Overall survival (OS), defined as the time from randomization to death from any cause
Objective response, defined as a complete response (CR) or partial response (PR) on two consecutive assessments, at least 28 days apart, as determined by investigator assessment using Response Evaluation Criteria in Solid Tumors (RECIST) version 1.1
Duration of objective response, defined as the time from first occurrence of a documented objective response to disease progression, as determined by investigator assessment using RECIST v1.1 or death from any cause, whichever occurs first.

Exploratory Efficacy Objectives

The exploratory efficacy objectives for this study are to evaluate the efficacy of the combination of trastuzumab emtansine plus atezolizumab compared with trastuzumab emtansine plus placebo on the basis of the following endpoints:
PFS, defined as the time from randomization to the first occurrence of disease progression, as determined by investigator assessment using RECIST v1.1 or death from any cause, whichever occurs first, in the programmed death-ligand 1 (PD-L1) selected subgroup of patients defined as having tumor immune infiltrating cell (IC) expression of IC 1/2/3, as assessed by immunohistochemistry
PFS, defined as the time from randomization to the first occurrence of disease progression, as determined by investigator assessment using immune-modified RECIST or death from any cause, whichever occurs first
Objective response, defined as a CR or PR on two consecutive assessments, at least 28 days apart, as determined by investigator assessment using immune modified RECIST
Duration of objective response, defined as the time from first occurrence of a documented objective response to disease progression, as determined by investigator assessment using immune-modified RECIST or death from any cause, whichever occurs first
1-year survival rate

Safety Objectives

The safety objectives for this study are to evaluate the overall safety of trastuzumab emtansine in combination with atezolizumab compared with trastuzumab emtansine in combination with placebo on the basis of the following:
Nature, frequency, severity, and timing of adverse events including cardiac, hepatic and pulmonary events
Clinical laboratory results during and following trastuzumab emtansine and atezolizumab administration

Pharmacokinetic Objectives

The pharmacokinetic (PK) objectives for this study are:
To characterize the pharmacokinetics of atezolizumab in the presence of trastuzumab emtansine
To characterize the pharmacokinetics of trastuzumab emtansine in the presence and absence of atezolizumab

Immunogenecity Objectives

The immunogenicity objectives for this study are:
To characterize the incidence of anti-therapeutic antibody (ATA) to atezolizumab in the presence of trastuzumab emtansine
To characterize the incidence of ATA to trastuzumab emtansine in the presence and absence of atezolizumab

Biomarker Objectives

The exploratory biomarker objectives for this study are as follows:
To assess if baseline PD-L1 expression is associated with efficacy
To assess if baseline immune status is associated with efficacy
To assess if baseline immune status together with HER2 expression level (mRNA, protein and/or gene copy number/ratio) are associated with efficacy
To assess changes in expression levels of biomarkers or biomarker panels during and after investigational treatment with atezolizumab in combination with trastuzumab emtansine
To evaluate the relationship between tumor biomarkers and efficacy
To identify candidate biomarkers that correlate with safety signals

Study Design

Description of the Study

This is a Phase II, randomized, multicenter, international, two-arm, double-blind, placebo-controlled clinical trial designed to compare the efficacy and safety of trastuzumab emtansine in combination with either atezolizumab or placebo for patients with HER2-positive locally advanced or MBC who have received prior trastuzumab and taxane based therapy.

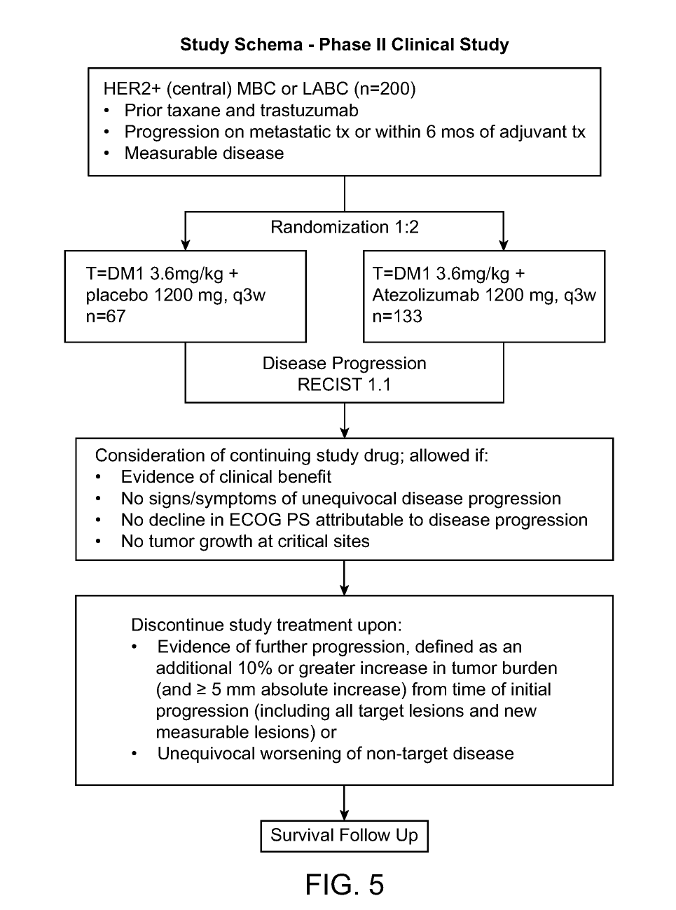

Approximately 200 patients will be enrolled in the study at 100 sites worldwide (FIG. 5). Patients will be randomized to treatment arms A and B in a 1:2 ratio by means of a permuted block randomization scheme through the use of an interactive Web or voice response system (xRS). Randomization will be stratified according to 1) PD-L1 status (IC0 vs IC1/2/3), 2) World Region (Western Europe vs U.S. vs Rest of World) and 3) Presence of liver metastases (yes vs. no).

Patients will be treated in one of the following arms:
Arm A: trastuzumab emtansine 3.6 mg/kg and placebo, every 3 weeks (q3w) (approximately 67 patients)
Arm B: trastuzumab emtansine 3.6 mg/kg and atezolizumab 1200 mg, q3w (approximately 133 patients)

Arm A and Arm B will be blinded with respect to administration of atezolizumab or placebo. Cross-over between treatment arms will not be permitted.

Patients must have measurable disease at baseline that is evaluable per RECIST 1.1. Patients must also have unresectable, locally advanced or metastatic disease. Locally advanced disease must not be amenable to resection or other local therapy with curative intent.

Tumor assessments will be conducted approximately every 6 weeks (±7 days) from the date of randomization, until investigator-assessed PD per RECIST 1.1 or death, whichever occurs first, regardless of dose delays or dose interruptions and even if study treatment has been discontinued as a result of patient or physician choice or unacceptable toxicity. Tumor assessment scans will be collected prospectively by the Sponsor in the event that an independent review facility (IRF) will be utilized.

Patients may remain on study treatment until investigator-assessed disease progression, unmanageable toxicity, or study termination by the Sponsor.

Patients who demonstrate control of their systemic disease, defined as having received clinical benefit (CR or PR of any duration or stable disease ≥4 months per RECIST v1.1) from study therapy, but who newly develop isolated brain metastases that are treatable (e.g., with surgery, radiation or gamma-knife) may continue with study treatment until they experience systemic progression of their disease or further progression in the brain or both, based on investigator assessment. Other requirements include the following:
The patient cannot miss more than one cycle (i.e., the maximum allowed time window between study treatments is 42 days) for the treatment of their brain disease.
The patient must have an Eastern Cooperative Oncology Group (ECOG) performance status of ≤2 to continue on study therapy.
Brain MRI or CT scans are performed along with regularly scheduled tumor assessments in these instances.
Every attempt should be made to discontinue corticosteroids within 2 weeks of the last day of radiation therapy.

Upon radiographic disease progression per RECIST v1.1, patients may optionally continue to receive trastuzumab emtansine with blinded atezolizumab or placebo, provided they meet the following criteria.
Evidence of clinical benefit as assessed by the investigator
Absence of symptoms and signs (including worsening of laboratory values [e.g., new or worsening hypercalcemia]) indicating clinically significant progression of disease
No decline in ECOG performance status that can be attributed to disease progression
Absence of tumor progression at critical anatomical sites (e.g., leptomeningeal disease) that cannot be managed by protocol-allowed medical interventions (i.e., pain secondary to disease or unmanageable ascites, etc.), as determined by the investigator after an integrated assessment of radiographic data, biopsy results (if available), and clinical status

Patients should discontinue study therapy upon evidence of further progression, defined as an additional 10% or greater increase in tumor burden (and ≥5 mm absolute increase) relative to the first scan that documented progressive disease (including target lesions and new measurable lesions) or an unequivocal worsening of non-target disease. For lymph node lesions, only increases in size beyond 15 mm (short axis) should be included in the calculation of tumor burden.

In patients who continue treatment beyond radiographic disease progression per RECIST v1.1, tumor response will also continue to be assessed using immune-modified RECIST criteria every 6 weeks (±7 days) until study treatment discontinuation. Immune-modified RECIST criteria account for the possibility of delayed anti-tumor activity that may be preceded by initial apparent radiological progression, including the appearance of new lesions.

For estimation of PFS, ORR, and DOR, tumor response will be based on both RECIST v1.1 and immune-modified RECIST.

Safety assessments will include the incidence, nature, and severity of adverse events and laboratory abnormalities graded per NCI CTCAE v4.0. Laboratory safety assessments will include the regular monitoring of hematology and blood chemistry.

Serum samples will be collected to monitor pharmacokinetics and to detect presence of antibodies to trastuzumab emtansine and atezolizumab. Patient samples, including tumor tissues, as well as serum and plasma and whole blood, will be collected for exploratory biomarker assessments.

After the Study Drug Completion Visit, all patients (regardless of reason for discontinuation) will be followed up for their survival status every 3 months until death, loss to follow-up, withdrawal of consent, or study termination by the Sponsor. After patients discontinue from study treatment, information on subsequent anti-cancer therapies will be collected according to the same schedule as survival follow-up.

The study schema is shown in FIG. 5,

End of Study and Length of Study

This study is anticipated to have a recruitment period of approximately 9 months. The final analysis of the primary efficacy endpoint will be conducted when approximately 90 PFS events have occurred, based on investigator assessments. This is assumed to be approximately 19 months after the enrollment of the first patient (FPI).

The end of study is triggered by the final OS analysis following last patient last visit (LPLV) which is planned to occur approximately 9 months after the primary efficacy analysis. The Sponsor may also terminate the study at any time.

### Example 5—Phase II Clinical Trial: Materials and Methods

Materials and Methods

Patients

The study will enroll patients with HER2-positive LABC or MBC who have received prior trastuzumab and taxane based therapy either alone or in combination and/or who have progressed within 6 months after completing adjuvant therapy. Patients must comply with the following inclusion and exclusion criteria.

Inclusion Criteria

Patients must meet ALL of the following inclusion criteria to be eligible for study entry:

Age ≥18 years.

Signed written informed consent approved by the institution's Independent Ethical Committee/Institutional Review Board (IRB).

Archival tumor samples must be obtained from primary and/or metastatic sites. Representative FFPE tumor specimens in paraffin blocks (blocks preferred) or at least 20 unstained slides, with an associated pathology report, for central testing is required; patients who have fewer than 20 unstained slides available at baseline (but no fewer than 15) may be eligible following discussion with the Medical Monitor. Tumor tissue should be of good quality based on total and viable tumor content and must be evaluated for HER2 and PD-L1 expression prior to enrollment.

Patients must submit tumor tissue that is evaluable for PD-L1 expression to be eligible for this study. If multiple tumor specimens are submitted (e.g., an archival specimen [from initial BC diagnosis] and tissue from metastatic or LABC disease), patients may be eligible if at least one specimen is evaluable for PD-L1.
For the purpose of stratification, the PD-L1 score of the patient will be the maximum PD-L1 score among the samples. Tumor tissue from bone metastases is not evaluable for PD-L1 expression and is therefore not acceptable.
Patients who do not have tissue specimens that meet eligibility requirements may undergo a biopsy during the screening period. Acceptable samples include core needle biopsies for deep tumor tissue (minimum three cores) or excisional, incisional, punch, or forceps biopsies for cutaneous, subcutaneous, or mucosal lesions. Fine-needle aspiration, brushing, cell pellet from pleural effusion, bone metastases, and lavage samples are not acceptable.

HER2-positive BC as defined by an IHC score of 3+ or gene amplified by in situ hybridization (ISH) as defined by a ratio of ≥2.0 for the number of HER2 gene copies to the number of chromosome 17 copies, prospectively tested by a Sponsor-designated central laboratory prior to enrollment. Both IHC and ISH assays will be performed; however, only one positive result is required for eligibility.
If multiple tumor specimens are submitted, the HER2 IHC score and or ISH amplification ratio will be taken from the analysis of the archival specimen for the purpose of determining eligibility. For patients with bilateral BC, HER2 positivity must be demonstrated in both locations for archival tissue or in a metastatic biopsy.

Histologically or cytologically confirmed invasive BC: incurable, unresectable, locally advanced BC previously treated with multimodality therapy or MBC.

Prior treatment for BC in the: adjuvant; unresectable locally advanced; or metastatic settings; which must include both, a taxane and trastuzumab (alone or in combination with another agent)

Progression must have occurred during or after most recent treatment for LABC/MBC or within 6 months after completing adjuvant therapy.

Patients must have measurable disease that is evaluable per RECIST 1.1.

ECOG Performance Status of 0 or 1.

Adequate hematologic and end-organ function, as evidenced by the following local laboratory results obtained within 2 weeks prior to the first study treatment (Cycle 1, Day 1):
Absolute neutrophil count ≥1500 cells/μL (without granulocyte-colony stimulating factor [support) within 2 weeks prior to Cycle 1, Day 1
Platelet count ≥100,000 /μL (without transfusion within 2 weeks prior to Cycle 1, Day 1)
Hemoglobin ≥9.0 g/dL Patients may be trasfused of receive erythropoietic treatment to meet this criterion.
Albumin >2.5g/dL
AST, ALT, and alkaline phosphatase ≤2.5×the upper limit of normal (ULN) with the following exceptions:
   Patients with documented bone metastases: alkaline phosphatase ≤5×the ULN
Total bilirubin ≤1.5×the ULN
INR and aPTT≤1.5×the ULN

This applies only to patients who are not receiving therapeutic anticoagulation; patients receiving therapeutic anticoagulation should be on a stable dose.
Calculated creatinine clearance ≤30 mL/min

Negative serum pregnancy test for pre-menopausal women and for women less than 12 months after the onset of menopause.

For women of childbearing potential: agreement to remain abstinent (refrain from heterosexual intercourse) or use contraceptive methods that result in a failure rate of <1% per year during the treatment period and for at least 7 months after the last dose of study drug.

A woman is considered to be of childbearing potential if she is postmenarcheal, has not reached a postmenopausal state (≥12 continuous months of amenorrhea with no identified cause other than menopause), and has not undergone surgical sterilization (removal of ovaries and/or uterus).

Examples of contraceptive methods with a failure rate of <1% per year include bilateral tubal ligation, male sterilization, {established, proper use of hormonal contraceptives that inhibit ovulation, hormone-releasing intrauterine devices}, and copper intrauterine devices.

The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.

For men: agreement to remain abstinent (refrain from heterosexual intercourse) or use contraceptive measures, and agreement to refrain from donating sperm, as defined below:

With female partners of childbearing potential, men must remain abstinent or use a condom plus an additional contraceptive method that together result in a failure rate of <1% per year during the treatment period and for at least 7 months after the last dose trastuzumab emtansine. Men must refrain from donating sperm during this same period.

With pregnant female partners, men must remain abstinent or use a condom during the treatment period and for at least 7 months after the last dose of trastuzumab emtansine to avoid exposing the embryo.

The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.

Exclusion Criteria

Patients who meet any ONE of the following criteria will be excluded from study entry:

Prior treatment with trastuzumab emtansine, CD137 agonists, anti-PD-1, or anti-PD-L1 therapeutic antibody or pathway-targeting agents.

Receipt of any anti-cancer drug/biologic or investigational treatment within 28 days prior to randomization except hormone therapy, which can be given up to 7 days prior to randomization; recovery of treatment-related toxicity consistent with other eligibility criteria.

Radiation therapy within 2 weeks prior to Cycle 1, Day 1. The patient must have recovered from any resulting acute toxicity (to Grade≤1) prior to randomization.

History of exposure to the following cumulative doses of anthracyclines as specified below:
Doxorubicin >500 mg/m²
Liposomal doxorubicin >500 mg/m²
Epirubucin >720 mg/m² Mitoxantrone >120 mg/m²
Idarubicin >90 mg/m² If another anthracycline or more than one anthracycline has been used, then the cumulative dose must not exceed the equivalent of 500 mg/m² doxorubicin. If another anthracycline or more than one anthracycline has been used, then the cumulative dose must not exceed the equivalent of 500 mg/m² of doxorubicin.

History of other malignancy within the previous 5 years, except for appropriately treated carcinoma in situ of the cervix, non-melanoma skin carcinoma, Stage I uterine cancer, or patients who have undergone potentially curative therapy with no evidence of disease and are deemed by the treating physician to be at low risk for recurrence.

Cardiopulmonary dysfunction as defined by:
Uncontrolled hypertension (systolic >150 mm Hg and/or diastolic >100 mm Hg)
Inadequate left ventricular ejection function at baseline, <50% by either ECHO or MUGA
History of symptomatic congestive heart failure (CHF)-Grade≥3 per NCI CTCAE version 4.0 or Class ≥II New York Health Association
History of a decrease in left ventricular ejection function to <40% or symptomatic CHF with prior trastuzumab treatment
Myocardial infarction or unstable angina within 6 months of randomization
Current dyspnoea at rest due to complications of advanced malignancy, or other disease requiring continuous oxygen therapy

Current severe, uncontrolled systemic disease (e.g., clinically significant cardiovascular, pulmonary or metabolic disease; wound healing disorders; ulcers; bone fractures).

Major surgical procedure or significant traumatic injury within 28 days prior to randomization or anticipation of the need for major surgery during the course of study treatment.

Clinically significant history of liver disease, including cirrhosis, current alcohol abuse, autoimmune hepatic disorders, sclerosis cholangitis or active infection with HIV, hepatitis B virus (HBV), or hepatitis C virus (HCV)
Active infection is defined as requiring treatment with antiviral therapy or presence of positive test results for hepatitis B (hepatitis B surface antigen and/or total hepatitis B core antibody) or HCV antibody. HIV, HBV, or HCV assessments are required at screening.
Patients who test positive for hepatitis B core antibody are eligible only if test results are also positive for hepatitis B surface antibody and polymerase chain reaction is negative for HBV DNA.
Patients who are positive for HCV serology are only eligible if testing for HCV RNA is negative.

Need for current chronic corticosteroid therapy (≥10 mg of prednisone per day or an equivalent dose of other anti-inflammatory corticosteroids) Stable use (i.e., no change in dose within 3 months prior to Cycle 1, Day 1) of inhaled corticosteroids is allowed.

Spinal cord compression not definitively treated with surgery and/or radiation, or previously diagnosed and treated spinal cord compression without evidence that disease has been clinically stable for >2 weeks prior to randomization.

Patients with known central nervous system (CNS) disease are not eligible, except for treated asymptomatic CNS metastases, provided that all of the following criteria are met:
Only supratentorial and cerebellar metastases allowed (i.e., no metastases to midbrain, pons, medulla, or spinal cord)
No ongoing requirement for corticosteroids as therapy for CNS disease
No stereotactic radiation within 14 days prior to randomization
No evidence of interim progression between the completion of CNS-directed therapy and the screening radiographic study

Note: Patients with new asymptomatic CNS metastases detected at the screening scan must receive radiation therapy and/or surgery for CNS metastases. Following treatment, these patients may be eligible without the need for an additional brain scan prior to enrollment, if all other criteria are met.

Symptomatic pleural effusion, pericardial effusion, or ascites.

Uncontrolled hypercalcemia (>1.5 mmol/L ionized calcium or calcium >12 mg/dL or corrected serum calcium greater than the ULN) or symptomatic hypercalcemia requiring continued use of bisphosphonate therapy.
Patients who are receiving denosumab must discontinue use of denosumab and replace it with a bisphosphonate instead while on study.
Patients who are receiving bisphosphonate therapy specifically to prevent skeletal events and who do not have a history of clinically significant hypercalcemia are eligible.

Current Grade≥3 peripheral neuropathy (according to the NCI CTCAE v4.0).

History of severe allergic, anaphylactic, or other hypersensitivity reactions to chimeric or humanized antibodies, excipients of any drugs formulated in polysorbate 80 or 20 or fusion proteins.

History of autoimmune disease, including, but not limited to, myasthenia gravis, autoimmune myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barré syndrome, multiple sclerosis, vasculitis, or glomerulonephritis.

Patients with a history of autoimmune-related hypothyroidism on a stable dose of thyroid replacement hormone may be eligible for this study.

History of inflammatory bowel disease (e.g., Crohn's disease or ulcerative colitis) or active bowel inflammation (e.g., diverticulitis).

Patients with Type 1 diabetes mellitus will not be eligible unless controlled with the patient on a stable insulin regimen

Patients with eczema, psoriasis, lichen simplex chronicus of vitiligo with dermatologic manifestations only (e.g., patients with psoriatic arthritis would be excluded) are excluded unless they meet the following conditions:
Rash must cover <10% of body surface area.
Disease is well controlled at baseline and requiring only low-potency topical steroids (e.g., hydrocortisone 2.5%, hydrocortisone butyrate 0.1%, flucinolone 0.01%, desonide 0.05%, aclometasone dipropionate 0.05%)
No acute exacerbations of underlying condition within the last 12 months (not requiring psoralen plus ultraviolet. A radiationmethotrexate, retinoids, biologic agents, oral calcineurin inhibitors, or high-potency or oral steroids)
Patients with psoriasis must have a baseline ophthalmologic exam to rule out ocular manifestations.

Prior Allogeneic Stem Cell or Solid Organ Transplantation.

History of idiopathic pulmonary fibrosis (including pneumonitis), drug-induced pneumonitis, organizing pneumonia (i.e., bronchiolitis obliterans, cryptogenic organizing pneumonia), or evidence of active pneumonitis on screening chest computed tomography (CT) scan. Patients with a history of radiation pneumonitis in the radiation field (fibrosis) are eligible.

Active Tuberculosis.

Receipt of a live, attenuated vaccine within 4 weeks prior to randomization or anticipation that such a live, attenuated vaccine will be required during the study.

Treatment with systemic immunostimulatory agents (including, but not limited to, interferons or IL-2) within 4 weeks or five half-lives of the drug (whichever is shorter) prior to randomization.

Treatment with systemic corticosteroids or other systemic immunosuppressive medications (including but not limited to prednisone, dexamethasone, cyclophosphamide, azathioprine, methotrexate, thalidomide, and anti-tumor necrosis factor [TNF] agents) within 2 weeks prior to randomization, or anticipated requirement for systemic immunosuppressive medications during the trial.
Patients who need current chronic corticosteroid therapy (≥10 mg of prednisone per day or an equivalent dose of other anti-inflammatory corticosteroids) will be excluded.
Patients who have received acute, low-dose, systemic immunosuppressant medications (e.g., a one-time dose of dexamethasone for nausea) may be enrolled in the study.
Stable use (i.e., no change in dose within 3 months prior to Cycle 1, Day 1) of inhaled corticosteroids is allowed

### Example 6—Phase II Clinical Trial: Study Treatment

Method of Treatment Assignment and Blinding

Number of Patients/Assignment to Treatment Groups

Approximately 200 patients will be enrolled in the study and randomized to treatment arms A and B in a 1:2 ratio (approximately 67 patients in Arm A and 133 patients in Arm B). An Interactive voice/Web response system (IxRS) will be utilized to collect patient screening information and to randomize eligible patients to either treatment Arm A or B. Patients will be randomized within 28 days from initiation of screening. Patients will be blinded as to whether they receive atezolizumab or placebo in combination with trastuzumab emtansine. Investigators and study team members will also be blinded.

A permuted block randomization scheme will be used to achieve balance in treatment assignment within the two treatment arms with respect to pre-specified stratification factors.

Patients who are randomized into this study will not be allowed to be re-randomized to receive a second course of study treatment.

Once a patient has been randomized into the study, the IxRS will be used to assign the kit numbers for study drugs to be dispensed at each treatment visit. It is important that the study drugs dispensed for each visit are the correct kit number, as assigned by the IxRS. This will ensure that drug use by dates and automatic study drug resupply to sites are managed appropriately via the IxRS.

Stratification

Randomization will be stratified based on the following two factors:

Tumor PD-L1 status (PD-L1 IC 0 vs. IC 1/2/3)

World Region (Western Europe vs U.S. vs. Res of World)

Liver Metastases (yes vs. no)

Investigational Medicinal Products

The investigational medicinal products (IMP) for this study are trastuzumab emtansine, atezolizumab, and placebo. Each will be labeled according to regulatory requirements in each country, as well as in accordance with International Conference of Harmonisation (ICH) Good Clinical Practice (GCP) guidelines, and will be labeled for investigational use only.

Formulation, Packaging, and Handling

Trastuzumab Emtansine and Atezolizumab

The formulation, packaging, and handling of trastuzumab emtanzine and atezolizumab should be performed as described in Example 2.

Placebo

The formulation of placebo is equivalent to atezolizumab but without the active agent.

Dosage, Administration, and Compliance

Trastuzumab Emtansine

Trastuzumab emtansine will be given at a dose of 3.6 mg/kg by IV infusion, q3w. The dose of trastuzumab emtansine will be administered on the basis of the patient's baseline weight. Weight will be measured at each visit and dose must be re-adjusted for weight changes ≥10% compared to the previous visit or baseline. Administration may be delayed to assess or treat adverse events. Dose reduction will be allowed, following the dose reduction levels provided in Table 16.

TABLE 16
Dose Modification Scheme for Trastuzumab Emtansine

| Dose Reduction Schedule | Dose Level (mg/kg, q3w) |
|---|---|
| Starting dose | 3.6 |
| First dose reduction | 3.0 |
| Second dose reduction | 2.4 |
| Requirement for further dose reduction | Discontinue treatment |

Once a dose has been reduced for adverse event(s), it must not be re-escalated. If trastuzumab emtansine is discontinued because of toxicity, it should not be re-administered.

If the timing of a protocol-mandated procedure, such as administration of trastuzumab emtansine, coincides with a holiday that precludes the procedure, the procedure should be performed within 3 business days of the scheduled date and, when possible, on the earliest following date with subsequent protocol-specified procedures rescheduled accordingly.

Guidelines on administration of first and subsequent infusions of trastuzumab emtansine are shown in the following Table 17.

TABLE 17
Administration of First and Subsequent Infusions of Trastuzumab Emtansine

| First Infusion | Subsequent Infusions |
|---|---|
| No premedication is administered. | Record patient's vital signs |
| Record patient's vital signs | If prior infusions were well tolerated, |
| Administer the initial dose as a 90 | subsequent doses may be administered as |
| minute intravenous infusion. | 30 minute infusions |
| Patients should be observed during the | Patient should be observed during the |
| infusion and for at least | infusions and for at least 30 minutes after |
| 90 minutes following the initial dose for | infusion. |
| fever, chills, or other infusion related | |
| reactions. | |
| The infusion rate should be slowed or | |
| interrupted if the patient develops infusion- | |
| related symptoms | |
| The infusion site should be closely | |
| monitored for possible subcutaneous | |
| infiltration during drug administration | |

Atezolizumab/Placebo

Patients will receive 1200 mg of atezolizumab/placebo administered by IV infusion q3w. Atezolizumab/placebo infusions will be administered according to the instructions outlined in Table 17. Guidelines for dosage modification and treatment interruption or discontinuation are provided in later sections.

Both trastuzumab emtansine and atezolizumab/placebo should be administered in a monitored setting where there is immediate access to trained personnel and adequate equipment and medicine to manage potentially serious reactions.

Any overdose or incorrect administration of study drug should be noted on the Study Drug Administration electronic Case Report Form (eCRF). Adverse events associated with an overdose or incorrect administration of study drug should be recorded on the Adverse Event eCRF.

Refer to Table 18 for guidelines on administration of first and subsequent infusions of atezolizumab.

TABLE 18
Administration of First and Subsequent Infusions of Atezolizumab

| First Infusion | Subsequent Infusions |
|---|---|
| No premedication is administered. | If patient experienced infusion-related |
| Record patient's vital signs (heart rate, | reaction during any previous infusion, |
| respiratory rate, blood pressure, and | premedication with antihistamines may be |
| temperature) within 60 minutes before | administered for Cycles ≥2 at the discretion |
| starting infusion. | of the treating physician. |
| Infuse (one vial in 250 mL NaCl) over 60 | Record patient's vital signs (heart rate, |
| (±15) minutes. | respiratory rate, blood pressure, and |
| Record patient's vital signs (heart rate | temperature) within 60 minutes before starting |
| respiratory rate, blood pressure, and | infusion. |
| temperature) during and after the infusion if | If the patient tolerated the first infusion well |
| clinically indicated | without infusion-associated adverse events, |
| Patients will be informed about the possibility | the second infusion may be administered |
| of delayed post-infusion symptoms and | over 30 (±10) minutes. Continue to record |
| instructed to contact their study physician if | vital signs within 60 minutes before starting |
| they develop such symptoms. | infusion and during and after the infusion if |
| | clinically indicated. |
| | If the patient had an infusion-related |
| | reaction during the previous infusion, |
| | the subsequent infusion must be |
| | delivered over 60 (±15) minutes. |
| | Record patient's vital signs (heart rate, |
| | respiratory rate, blood pressure, and |
| | temperature) during the infusion if |
| | clinically indicated. |

Sequence of Drug Administration

All the study drugs are to be administered to patients intravenously. Atezolizumab or placebo will be administered first followed by trastuzumab emtansine.

Concomitant Therapy

Concomitant therapy includes any medication (e.g., prescription drugs, over-the-counter drugs, herbal or homeopathic remedies, nutritional supplements) used by a patient from 7 days prior to screening to the study treatment discontinuation visit. All such medications must be reported to the investigator and recorded on the Concomitant Medications eCRF.

Patients who use oral contraceptives, hormone-replacement therapy, or other maintenance therapy should continue their use.

Patients must be instructed not to take any concomitant medications (over-the-counter or other products) during the study without prior consultation with the investigator.

Permitted Therapy

The following therapies are permitted as concomitant medications in the study:
Prophylactic or therapeutic anticoagulation therapy (such as low-molecular weight heparin or warfarin at a stable dose level)
Palliative radiotherapy is permitted to treat pre-existing bone metastases only
Inactive influenza vaccinations during influenza season
Megestrol administered as an appetite stimulant
Inhaled corticosteroids for chronic obstructive pulmonary disease
Mineralocorticoids (e.g., fludrocortisone)
Low-dose corticosteroids for patients with orthostatic hypotension or adrenocortical insufficiency
Bisphosphonates for prevention of skeletal related events

In general, investigators should manage patient's care with supportive therapies as clinically indicated and per local standards. No protocol specified pre-medication with steroids for the first infusion trastuzumab emtansine or atezolizumab/placebo is required.

If pre-medication with steroids is being considered, please contact the Medical Monitor for approval.

Patients who experience infusion-associated symptoms may be treated symptomatically with acetaminophen, ibuprofen, diphenhydramine, and/or famotidine or another H2-receptor antagonist per standard practice (for sites outside the United States, equivalent medications may be substituted per local practice). Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists).

Prohibited Therapy

The following medications are prohibited while a patient is receiving study treatment:
Traditional herbal medicines: These therapies are not fully studied and their use may result in unanticipated drug-drug interactions that may cause or confound the assessment of toxicity.
Concomitant use of potent cytochrome (CYP) P450 3A4/5 inhibitors (such as ketoconazole and itraconazole) with trastuzumab emtansine should be avoided. Consider an alternate medication with no or minimal potential to inhibit CYP3A4/5. If a strong CYP3A4/5 inhibitor needs to be co-administered with trastuzumab emtansine, patients should be closely monitored for adverse reactions.
Excessive alcohol intake should be avoided (occasional to moderate use is permitted).
RANKL inhibitor (denosumab): Patients who are receiving denosumab prior to enrollment must be willing and eligible to receive a bisphosphonate instead while on study.
Immunomodulatory agents, including, but not limited to, interferons or IL-2, during the entire study; these agents could potentially increase the risk for autoimmune conditions when received in combination with atezolizumab.
Immunosuppressive medications, including, but not limited to, cyclophosphamide, azathioprine, methotrexate, and thalidomide; these agents could potentially alter the activity and the safety of atezolizumab.
Use of steroids to premedicate patients for whom CT scans with contrast are contraindicated (i.e., patients with contrast allergy or impaired renal clearance); in such patients, MRI scans of the chest, abdomen, and pelvis with a non-contrast CT scan of the chest must be performed.
Any live, attenuated vaccine (e.g., FluMist®) at any time during the study

Systemic corticosteroids and anti-TNF-α agents may also attenuate potential beneficial immunologic effects of treatment with atezolizumab but may be administered at the discretion of the treating physician. If feasible, alternatives to these agents should be considered.

In addition, patients should not receive other immunomodulatory agents for 10 weeks after atezolizumab discontinuation.

### Example 7—Phase II Clinical Trial: Study Assessments

Informed Consent Forms and Screening Log

Signed, written informed consent for participation in the study must be obtained before performing any study-related procedures. Informed Consent Forms for enrolled patients and for patients who are not subsequently enrolled will be maintained at the study site.

All screening evaluations must be completed and reviewed to confirm that patients meet all eligibility criteria before randomization into the study. The investigators will maintain a screening log to record details of all patients screened and to confirm eligibility or document reasons for screening failure, as applicable.

Medical History and Demographic Data

Medical history includes prior cancer therapies and procedures, reproductive status, smoking history, use of alcohol and drugs of abuse, and all medications

(e.g., prescription drugs, over-the-counter drugs, herbal or homeopathic remedies, and nutritional supplements) used by the patient within 7 days prior to the Cycle 1, Day 1 visit.

BC history includes prior cancer therapies and procedures.

Demographics will include age, gender, and self-reported race/ethnicity. Local HER2 testing information will also be collected.

Physical Examinations

A complete physical examination should be performed at screening and should include an evaluation of the head, eyes, ears, nose, and throat, and the cardiovascular, dermatological, musculoskeletal, respiratory, gastrointestinal, genitourinary, and neurological systems. Any abnormality identified at baseline should be recorded on the General Medical History and Baseline Conditions eCRF.

At subsequent visits (or as clinically indicated), limited, symptom-directed physical examinations should be performed. Changes from baseline abnormalities should be recorded in patient notes. New or worsened clinically significant abnormalities should be recorded as adverse events on the Adverse Event eCRF.

Vital Signs

Vital signs will include measurements of respiratory rate, pulse rate, systolic and diastolic blood pressure while the patient is in a seated position, and temperature.

Tumor and Response Evaluations

All sites of measurable and non-measurable disease must be documented at screening and re-assessed at each subsequent tumor evaluation. Tumor assessments will continue until disease progression, withdrawal of consent, death, or study termination by the Sponsor, whichever occurs first.

Initial screening assessments must include CT scans (with oral or IV contrast unless contraindicated) or MRI scans of the chest, abdomen, and pelvis. A bone scan or positron emission tomography (PET) scan should also be performed to evaluate for bone metastases. MRI scans of the chest, abdomen, and pelvis or non-contrast CT scan may be used in patients for whom CT scans with contrast are contraindicated (i.e., patients with contrast allergy or impaired renal clearance).

A CT (with contrast) or MRI scan of the head must be performed at screening to evaluate CNS metastasis in all patients. A MRI scan of the brain is required to confirm or refute a diagnosis of CNS metastasis at screening in the event of an equivocal scan. Patients with active or untreated CNS metastasis are not eligible for this study (See Example 5 for CNS-related exclusion criteria).

If a CT scan for tumor assessment is performed as part of a PET/CT, the CT acquisition must be consistent with the standards for a full-contrast diagnostic CT scan.

CT scans of the neck should also be performed if clinically indicated during the screening period. At the investigator's discretion, other methods of assessment of measurable disease according to RECIST v1.1 may be used.

After baseline tumor assessments, evaluation of tumor response conforming to RECIST v1.1 and immune-modified RECIST will be performed every 6 weeks (±7 days) following randomization, with additional scans performed as clinically indicated. The same radiographic procedures used to assess measurable disease sites at screening should be used throughout the study (e.g., the same contrast protocol for CT and/or MRI scans).

All known sites of disease must be documented at screening and re-assessed at each subsequent tumor evaluation. Response will be assessed by the investigator using RECIST v1.1 and immune-modified RECIST at each tumor assessment. Assessments should be performed by the same evaluator, if possible, to ensure internal consistency across visits.

At the investigator's discretion, CT or other clinically appropriate scans may be repeated at any time if PD is suspected. If the initial screening bone scan or PET scan does not show evidence of bone metastases, then these procedures do not need to be repeated unless clinically indicated or at the treating physician's discretion. Similarly, brain CT or MRI only need to be repeated beyond screening, if clinically indicated. In cases where a patient demonstrates control of their systemic disease but who newly develops isolated brain metastases and is eligible to remain on study treatment, brain MRI or CT are performed along with regularly scheduled tumor assessments (see Example 4).

If study drug treatment is discontinued prior to disease progression according to RECIST v1.1, tumor response assessment should continue to be performed.

In patients who continue treatment beyond radiographic disease progression per RECIST v1.1, tumor response will also continue to be assessed using immune-modified RECIST criteria, until study treatment discontinuation.

All primary imaging data used for tumor assessment will be collected by the Sponsor to enable centralized, independent review of response endpoints, if needed.

Left Ventricular Ejection Fraction Assessment

Left Ventricular Ejection Fraction (LVEF) will be assessed by ECHO or MUGA. LVEF will be monitored at baseline, and on Day 15-21 of Cycle 1, and every fourth cycle thereafter. Additional LVEF measurements may be performed if LVEF declines are clinically suspected at the discretion of the investigator.

Electrocardiogram

A 12-lead ECG is required at screening and as clinically indicated.

ECGs for each patient should be obtained from the same machine wherever possible. ECG recordings must be performed after the patient has been resting in a supine position for at least 10 minutes.

For safety monitoring purposes, the investigator must review, sign, and date all ECG tracings. Paper copies of ECG tracings will be kept as part of the patient's permanent study file at the site. Any morphologic waveform changes or other ECG abnormalities must be documented on the eCRF.

Laboratory, Biomarker, and Other Biological Samples

Laboratory Samples

Samples obtained from the following laboratory tests will be sent to the study site's local laboratory for analysis:
Hematology (CBC, including RBC count, hemoglobin, hematocrit, WBC count with differential [neutrophils, eosinophils, lymphocytes, monocytes, basophils, and other cells], and platelet count)
Serum chemistry (glucose, BUN or urea, creatinine, sodium, potassium, magnesium, chloride, bicarbonate, calcium, phosphorus, total bilirubin, ALT, AST, alkaline phosphatase, total protein, and albumin)
Coagulation (aPTT and INR)
Serum pregnancy test for women of childbearing potential, including women who have had a tubal ligation; urine pregnancy tests will be performed every third cycle during treatment. If a urine pregnancy test is positive, it must be confirmed by a serum pregnancy test. Childbearing potential is defined as not having undergone surgical sterilization, hysterectomy, and/or bilateral oophorectomy or not being post-menopausal (≥12 months of amenorrhea).
Urinalysis (specific gravity, pH, glucose, protein, ketones, and blood)
Thyroid function test (thyroid-stimulating hormone [TSH], free T3, and free T4)
HIV (tested prior to patient enrollment in the study) HIV-positive patients are excluded from study participation.
HBV serology (HBsAg, antibody to HBsAg [anti-HBs], and anti-HBc) HBV DNA testing is required prior to or on Day 1 of Cycle 1 if a patient has negative serology for HBsAg and positive serology for anti-HBc. HCV serology (anti-HCV)

The assessments listed below will be performed at a central laboratory or by the Sponsor. Any remaining material from samples collected to enable these central assessments may be used for additional related safety assessments (e.g., ATA assay), exploratory biomarker profiling, and pharmacodynamic assay development purposes. Instruction manuals and supply kits will be provided for all central laboratory assessments.
Central HER2 testing (for eligibility assessment) and PD-L1 testing (for stratification)
Tumor and blood samples for RNA and DNA extraction for gene expression and genomic sequencing. If gene expression or genomic sequencing is performed, samples will be sent to one or more laboratories for analysis.
Biomarker blood assays: Blood samples will be processed to obtain blood cells, plasma, and serum for the determination of baseline level changes in surrogate pharmacodynamics biomarkers
Epstein-Barr virus serology (screening sample collection only; serology tests to be performed only in patients who experience an acute inflammatory event such as systemic inflammatory response syndrome (SIRS) while receiving study treatment)
C-reactive protein
Serum HER2 extra cellular domain at baseline
ATA assays: serum samples will be assayed for the presence of ATAs to atezolizumab and trastuzumab emtansine using validated immunoassays.
Auto-antibody testing: The baseline sample will be collected on Cycle 1, Day 1, prior to the first dose of study drug. For patients who show evidence of immune-mediated toxicity, additional samples may be collected and will be analyzed centrally.
   Anti-nuclear antibody
   Anti-double-stranded DNA
   Circulating anti-neutrophil cytoplasmic antibody
   Perinuclear anti-neutrophil cytoplasmic antibody
PK assays: Serum samples will be assayed for atezolizumab, trastuzumab emtansine, and total trastuzumab concentrations using validated immunoassays. Plasma samples will be assayed for DM1 concentration using a validated liquid chromatography-tandem mass spectrometry.

Biomarker Samples

Whole blood samples will be taken from all patients enrolled on the study. A blinded interim biomarker analysis will be conducted on the first 40 enrolled patients and further analysis will be gated based on the interim biomarker analysis.

After completion of HER2 and PD-L1 testing, patient samples may also be tested with other exploratory assays/technologies to establish performance characteristics of these assays for both next generation diagnostic development and understanding treatment response associated to characteristics of the tumor microenvironment. Testing could be performed on all screened patients (screen-failed and enrolled) and will be performed only after eligibility is established for each patient. These exploratory testing data will have no impact on patient eligibility.

After initial HER2 and mandatory biomarker testing, the tissue blocks will be used for midterm storage up to the time of final clinical study report before returning them to sites to allow for future biomarker analysis. Hereafter, the blocks will be shipped back, unless the patient gives specific consent for the remainder of the samples to be stored for optional exploratory research. In cases where only slides were sent because of country or site regulations, a new request of slides will be sent to the sites in case additional markers or assays are defined up to final clinical study report. The midterm storage of tissue and the possibility of requesting more slides up to the time of the final clinical study report extends the possibility of deciding on analyses of important additional markers or assays while the study is ongoing or until final study data are available.

In cases where midterm storage of tumor tissue blocks is not allowed by local regulatory bodies (including IRB/EC policies) for patients without consent to RBR, the block will be sent back to the site no later than 3-6 months and requested at a later timepoint within the study in case additional analyses are defined. If the patient provides consent for optional exploratory research (RBR), the samples will be stored until no longer required in accordance with the IRB/EC-approved Informed Consent Form and applicable laws (e.g., health authority requirements). If tissue material from more than one timepoint was submitted, the midterm storage only applies to the most recent sample and the archival tumor tissue block for all patients enrolled will be returned no later than 3-6 months after eligibility determination. In case archival partial blocks or slides are sent, this tissue will not be returned.

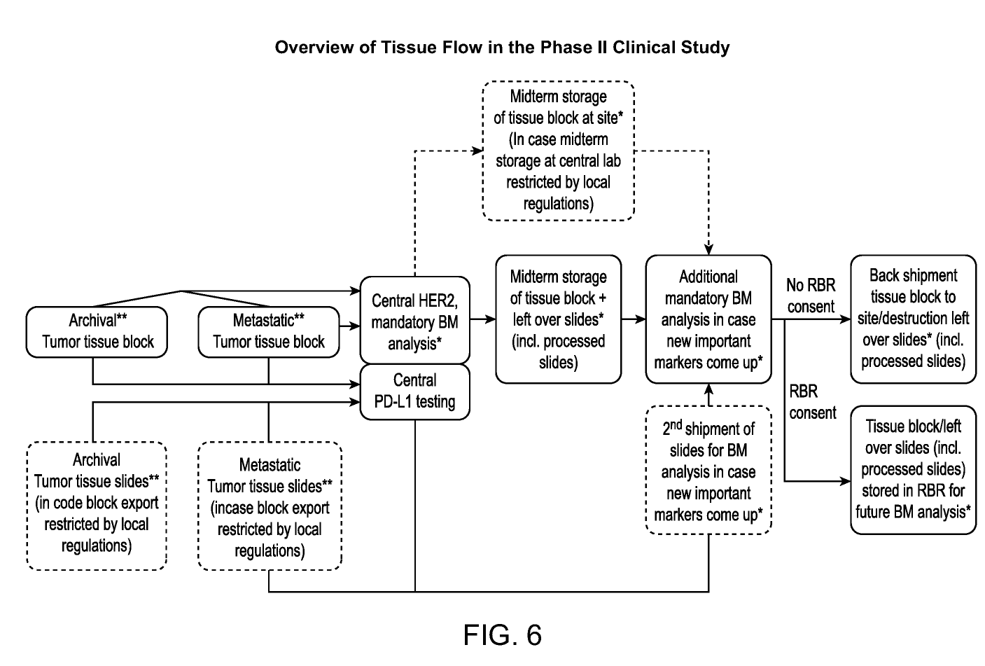

All tissue blocks from patients who are not eligible to enroll in the study will be returned no later than 3-6 months after eligibility determination. FIG. 6 gives an overview of the tissue flow in the trial.

All biomarker samples taken during the study are summarized in Table 19, Exploratory biomarker research may include, but will not be limited to, the biomarkers listed in Table 19. Such biomarker research may be required as science is rapidly and constantly evolving. Therefore, a definitive list of analyzed biomarkers may include additional parameters as well as novel or alternative technologies.

TABLE 19
Proposed Biomarkers for Exploratory Research

| Sample Type | Timing | Biomarker |
|---|---|---|
| Breast Cancer Tumor | Mandatory | HER2 |
| tissue | Baseline (archival | PD-L1 |
| | and/or metastatic) | CD8 |
| | Optional Baseline (freshly | Tumor Infiltrating Lymphocytes |
| | taken) | |
| | Cycle 2 (optional), time of | |
| | progression (mandatory, if | |
| | clinically feasible) | |
| DNA/RNA extracted from | Baseline, Cycle 2 and time | Immune and cancer associated |
| breast cancer tissue | of progression (if biopsy | gene signatures (RNA) |
| | clinically feasible) | Cancer related genes (DNA) |
| Plasma | Baseline and | HER ligands |
| | subsequent timepoints | |
| | during treatment | |
| Serum | Baseline and | Cytokines (e.g., IL-2, IFNg, IL-18) |
| | subsequent timepoints | |
| | during treatment | |
| DNA extracted from | Baseline (or later if missed | Polymorphisms in: |
| Whole Blood | at baseline) | PD-L1, PD-1 |
| | | IL-8, IL-6, and related cytokines |
| | | Immune genes (NGS) |
| | | Cancer related genes (NGS) |
| Circulating tumor DNA | Baseline and | ctDNA HER2 |
| isolated from plasma | subsequent timepoints | ctDNA PIK3CA |
| | during treatment | |
| RNA extracted from | Baseline and subsequent | Immune gene expression profiles |
| Peripheral blood | timepoints during and after | (e.g. PD-L1, PD-1) |
| mononuclear cells | treatment | |
| (PBMCs) isolated from | | |
| whole blood | | |

HER2 = human epidermal growth factor 2; IL = interleukin; NGS = next-generation sequencing; PD-1 = programmed death-1; PD-L1 = programmed death-ligand 1.
^{a} Biomarker Interim Analysis on first 40 patients and further analysis gated on interim blinded results

Tumor Tissue Samples

After completion of HER2 and PD-L1 testing, patient samples may also be tested with other exploratory assays/technologies to establish performance characteristics of these assays for both next generation diagnostic development and understanding treatment response associated to characteristics of the tumor microenvironment. Testing could be performed on all screened patients (screen-failed and enrolled) and will be performed only after eligibility is established for each patient. These exploratory testing data will have no impact on patient eligibility.

After initial HER2 and mandatory biomarker testing, the tissue blocks will be used for midterm storage up to the time of final clinical study report before returning them to sites to allow for future biomarker analysis. Hereafter, the blocks will be shipped back, unless the patient gives specific consent for the remainder of the samples to be stored for optional exploratory research. In cases where only slides were sent because of country or site regulations, a new request of slides will be sent to the sites in case additional markers or assays are defined up to final clinical study report. The midterm storage of tissue and the possibility of requesting more slides up to the time of the final clinical study report extends the possibility of deciding on analyses of important additional markers or assays while the study is ongoing or until final study data are available.

In cases where midterm storage of tumor tissue blocks is not allowed by local regulatory bodies (including IRB/EC policies) for patients without consent to RBR, the block will be sent back to the site no later than 3-6 months and requested at a later timepoint within the study in case additional analyses are defined. If the patient provides consent for optional exploratory research (RBR), the samples will be stored until no longer required in accordance with the IRB/EC approved Informed Consent Form and applicable laws (e.g., health authority requirements).

If tissue material from more than one timepoint was submitted, the midterm storage only applies to the most recent sample and the archival tumor tissue block for all patients enrolled will be returned no later than 3-6 months after eligibility determination. In case archival partial blocks or slides are sent, this tissue will not be returned.

All tissue blocks from patients who are not eligible to enroll in the study will be returned no later than 3-6 months after eligibility determination.

FIG. 6 gives an overview of the tissue flow in the trial.

DNA and RNA from collected tumor tissue will be extracted and may enable targeted sequencing or whole exome sequencing (NGS and gene expression based methods) for exploratory research (that may include, but is not limited to, immune or cancer-related genes, PIK3CA mutation, mutational load and biomarkers associated with common molecular pathways).

NGS may be conducted by Foundation Medicine on samples collected at time of disease progression. If performed by Foundation Medicine, the investigator can obtain results from the samples collected at the time of disease progression in the form of an individualized report per patient, which is available upon request directly from Foundation Medicine. The investigator may share and discuss the results with the patient, unless the patient chooses otherwise. NGS may be performed by Foundation Medicine. If performed by Foundation Medicine, the investigator can obtain results from the samples collected at the time of disease progression in the form of an NGS report, which is available upon request directly from Foundation Medicine. The investigator may share and discuss the results with the patient, unless the patient chooses otherwise. The Foundation Medicine NGS assay has not been cleared or approved by health authorities. The NGS report is generated for research purposes and is not provided for the purpose of guiding future treatment decisions.

Blood Samples

Blood samples are taken for cells, plasma, and serum collection (Table 18). Whole blood samples may be analyzed by fluorescence-activated cell sorting and processed to obtain peripheral blood mononuclear cells (PBMCs) and their derivatives (e.g., proteins, RNA and DNA). Whole blood samples for PBMC collection will be taken on first 40 patients and collection of additionally enrolled patients will be gated based on potential changes seen in first 40 patients (blinded analysis).

Blood samples collected during the study may be evaluated for immune-related, tumor type-related, and other exploratory biomarkers (e.g., genetic alterations determined by DNA sequencing methods which may include NGS, and/or alterations in gene expression.

### Example 8—Phase II Clinical Trial: Patient, Treatment, Study, and Site Discontinuation, Assessment of Safety

Patient Discontinuation

Patients have the right to voluntarily withdraw from the study at any time for any reason. In addition, the investigator has the right to withdraw a patient from the study at any time. Reasons for withdrawal from the study may include, but are not limited to, the following:
Patient withdrawal of consent at any time (and for any reason)
Any medical condition that the investigator or Sponsor determines may jeopardize the patient's safety if he or she continues on the study
Investigator or Sponsor determines it is in the best interest of the patient to discontinue from the study
Patient non-compliance

Every effort should be made to obtain information on patients who withdraw from the study. The primary reason for withdrawal from the study should be documented on the appropriate eCRF. However, patients will not be followed for any reason after consent has been withdrawn. Patients who withdraw from the study will not be replaced.

Study Treatment Discontinuation

Patients must discontinue study treatment if they experience any of the following:
Intolerable toxicity related to study treatment
Any medical condition that may jeopardize the patient's safety if he or she continues on study treatment
Use of another systemic anti-cancer therapy
Pregnancy
Radiographic disease progression according to RECIST v1.1, with the following exception:
Patients who are randomized to receive atezolizumab and trastuzumab emtansine combination treatment may receive open-label atezolizumab, with or without trastuzumab emtansine until unacceptable toxicity or loss of clinical benefit, provided they meet all of the criteria specified above. Patients must provide written consent to acknowledge deferring any standard treatment options that may exist in favor of continuing study treatment at the time of initial progression.

The primary reason for study drug discontinuation should be documented on the appropriate eCRF.

Patients who discontinue study treatment prematurely will not be replaced.

Assessment of Safety

Safety Plan

The safety plan has been developed considering the risk measures for each IMP as well as the potential overlapping toxicities. While the safety profile of trastuzumab emtansine is generally well understood given its approval for treatment of HER2-positive LABC and MBC in patients treated previously with trastuzumab and or a taxane, atezolizumab is currently in clinical development and human experience is currently limited with the entire safety profile of atezolizumab is not known at this time. The safety considerations are based on results from nonclinical and ongoing clinical studies and published data on similar molecules.

Several measures will be taken to ensure the safety of patients participating in this study. Eligibility criteria, as described earlier, have been designed to exclude patients at higher risk for toxicities from study participation. Patients will undergo safety monitoring by an iDMC during the study, including assessment of the nature, frequency, and severity of adverse events; details of this safety monitoring will be specified in the iDMC Charter. In addition, guidelines for managing adverse events, including criteria for dosage modification and treatment interruption or discontinuation, are provided below.

Please refer to the latest versions of the trastuzumab emtansine and atezolizumab IBs for a complete summary of safety information.

Risks Associated with Trastuzumab Emtansine

Pulmonary Toxicity

Cases of interstitial lung disease (ILD), including pneumonitis, some leading to acute respiratory distress syndrome or death, have been reported in patients receiving trastuzumab emtansine. Signs and symptoms may include dyspnea, cough, fatigue, and pulmonary infiltrates. Patients with dyspnea at rest due to complications of advanced malignancy and comorbidities may be at risk of pulmonary events.

Patients who have experienced a pulmonary event should be carefully evaluated before commencing trastuzumab emtansine treatment.

Mild to moderate events of pneumonitis have been reported with atezolizumab. All pulmonary events should be thoroughly evaluated for other commonly reported etiologies such as pneomina/infection, lymphangitic carcinomatous, pulmonary embolism, heart failure, chronic obstructive pulmonary disease, or pulmonary hypertension. Management guidelines are provided in Table 20 below.

TABLE 20
Management Guidelines for Pulmonary Events and Pneumonitis
(Includes Interstitial Lung Disease)

| | | Trastuzumab |
|---|---|---|
| Severity | Atezolizumab | Emtansine |
| Grade 1 | May continue atezolizumab treatment with close monitoring | Discontinue |
| | Resume at same dose level when recovery of symptoms to | trastuzumab |
| | Grade 0 | emtansine |
| | Re-evaluate on serial imaging | treatment. |
| | Consider pulmonary consultation. | |
| Grade 2 | Withhold atezolizumab dose. | Discontinue |
| | Pulmonary and ID consultation with consideration | trastuzumab |
| | for bronchoscopy/BAL | emtansine |
| | Consider starting 60 mg prednisone or equivalent per day. | treatment. |
| | When improves to Grade 0 or Grade 1, then taper | |
| | steroids over ≥1 month. | |
| | Atezolizumab treatment may be resumed if the event improves | |
| | to Grade 0 or Grade 1 within 12 weeks, and corticosteroids have | |
| | been reduced to the equivalent of oral prednisone 10 mg daily or | |
| | less. | |
| | Treat as Grades 3-4 if recurrent episode of pneumonitis. | |
| Grade 3-4 | Permanently discontinue atezolizumab treatment | Discontinue |
| | Bronchoscopy/BAL is recommended. | trastuzumab |
| | Start 60 mg prednisone or equivalent per day. | emtansine |
| | Taper steroids over ≥1 month after symptoms improve to Grade | treatment. |
| | 0 or Grade 1. | |
| | If not improving after 48 hours or worsening, add additional | |
| | immunosuppression (e.g., infliximab, cyclophosphamide, IVIG, | |
| | or mycophenolate mofetil). | |
| | Contact the Medical Monitor if atezolizumab treatment | |
| | is discontinued. | |

BAL = broncho-alveolar lavage.

Hepatotoxicity

The following events have been reported with administration of trastuzumab emtansine:
Serious hepatobiliary disorders
   Serious hepatobiliary disorders, including nodular regenerative hyperplasia (NRH) of the liver and hepatobiliary disorders with a fatal outcome due to drug-induced liver injury, have been observed in patients treated with trastuzumab emtansine. Some of the observed cases may have been confounded by concomitant medications with known hepatotoxic potential.
Increased serum transaminases
   Asymptomatic increases in serum transaminase concentration (transaminitis) have been observed. Grade 1 and 2 events have been observed frequently; Grade 3 and 4 events have been observed less commonly. The incidence of increased AST was substantially higher than that for increased ALT. Increases in AST and ALT were commonly observed by Day 8 of each cycle and generally improved or returned to baseline by Day 21. A cumulative effect of trastuzumab emtansine, that is, an increase in the proportion of patients with Grade 1 or 2 elevations in transaminases with successive cycles has been observed; however, there was no increase in the proportion of patients with Grade 3 abnormalities over time.
NRH
   Cases of NRH have been identified from liver biopsies in patients treated with trastuzumab emtansine who presented with signs and symptoms of portal hypertension. NRH is a rare liver condition characterized by widespread benign transformation of hepatic parenchyma into small regenerative nodules. NRH may lead to non-cirrhotic portal hypertension. Diagnosis of NRH can only be confirmed by histopathology. Biopsy-confirmed NRH leading to fatal hepatic failure has been reported. NRH should be considered in all patients with clinical symptoms of portal hypertension, even with normal transaminases, and no other manifestations of cirrhosis; in patients with a cirrhosis-like pattern seen on a CT scan of the liver; and/or in patients with liver failure following long-term treatment with trastuzumab emtansine.

Patients must meet specified hepatic laboratory test requirements to be included in this study, as discussed earlier.

Hepatic laboratory parameters will be monitored following a prescribed schedule of assessments.

Guidelines for management of trastuzumab emtansine in patients who develop increased serum transaminases, increased serum bilirubin, or NRH are as follows.

Immune-mediated hepatitis has been associated with the administration of atezolizumab. Eligible patients must have adequate liver function, as manifested by measurements of total bilirubin and hepatic transaminases. Liver function will be monitored throughout study treatment.

While on this study, patients who present with right upper-quadrant abdominal pain and/or unexplained nausea or vomiting should have liver function tests (LFTs) performed immediately and reviewed before administration of the next dose of study drug.

If outcome of LFTs is worsening, concurrent medications, viral hepatitis, and toxic or neoplastic etiologies should be considered and addressed, as appropriate. Imaging of the liver, gall bladder, and biliary tree should be performed to rule out neoplastic or other causes for worsening outcome of LFTs. Anti-nuclear antibody, perinuclear anti-neutrophil cytoplasmic antibody, anti-liver kidney microsomal antibodies, and anti-smooth muscle antibody tests should be performed if an autoimmune etiology is considered. See Table 21 for management guidelines for atezolizumab and trastuzumab emtansine hepatic events.

Note: No dose modification is indicated on the basis of hyperbilirubinemia alone.

TABLE 21
Management Guidelines for Increased Transaminases (AST/ALT) and Hepatic Events

| Severity | Atezolizumab | Trastuzumab Emtansine |
|---|---|---|
| ALT or AST | Discontinue atezolizumab treatment. | Discontinue |
| increase that meets | | trastuzumab emtansine |
| Hy's law criteria: | | treatment. |
| ALT or AST >3 × | | |
| ULN in combination | | |
| with TBILI >2 × | | |
| ULN or clinical | | |
| jaundice | | |
| ALT or AST | Treat at the same dose level. | Treat at the same dose |
| increase that | Continue LFT monitoring. | level. |
| does not meet | | |
| Hy's law criteria | | |
| ALT/AST Grade 1 | | |
| (>1-0-3.0 × ULN) | | |
| ALT/AST Grade 2 | Withhold atezolizumab dose. | Treat at the same dose |
| (>3.0-5.0 × ULN) | If persists >5-7 days: Consider starting | level. |
| | 60 mg prednisone or equivalent per day; | |
| | when recover to ≤Grade 1, taper steroids | |
| | over ≥1 month. | |
| | Resume therapy when systemic steroid | |
| | dose is ≤10 mg oral prednisone equivalent | |
| | per day and resume when recovery to ≤Grade | |
| | 1 at same dose. | |
| ALT/AST Grade 3 | Discontinue atezolizumab dose. | Withhold trastuzumab |
| (>5.0-20.0 × ULN) | Consider GI consult and liver biopsy | emtansine dose. |
| | to establish etiology of hepatic injury if | Do not administer |
| | necessary. | trastuzumab emtansine |
| | Start 60 mg prednisone or equivalent | until recovery |
| | per day. | to ≤Grade 2, and then |
| | If LFT results do not decrease within | resume with dose |
| | 48 hours after initiation of systemic | reduction by one level. |
| | steroids, addition of an alternative | Discontinue |
| | immunosuppressive agent (e.g., | trastuzumab emtansine |
| | mycophenolate or TNF-α antagonist) may | treatment if the event |
| | be considered. | has not resolved |
| | Taper steroids over ≥1 month, when | to ≤Grade 2 within 42 |
| | symptoms improve to Grade 0 or Grade 1. | days after the last dose |
| | Contact the Medical Monitor if atezolizumab | received. |
| | treatment is discontinued. | |
| ALT/AST Grade 4 | Discontinue atezolizumab treatment. | Discontinue |
| (>20.0 × ULN) | Consider GI consult and liver biopsy to | trastuzumab |
| | establish etiology of hepatic injury if | emtansine treatment. |
| | necessary. | Laboratory tests may |
| | Start 60 mg prednisone or equivalent per | be repeated (within |
| | day. | 24 hours) to exclude |
| | If LFT results do not decrease within | laboratory error prior |
| | 48 hours after initiation of systemic steroids, | to discontinuing |
| | addition of an alternative immunosuppressive | trastuzumab |
| | agent (e.g., mycophenolate or TNF-α | emtansine. |
| | antagonist) may be considered. | |
| | Taper steroids over ≥1 month, when | |
| | symptoms improve to Grade 0 or Grade 1. | |
| | Contact the Medical Monitor if atezolizumab | |
| | treatment is discontinued. | |
| NRH | Discontinue atezolizumab treatment | Discontinue |
| If there are signs of | | trastuzumab |
| portal hypertension | | emtansine treatment |
| (e.g., ascites and/or | | and have the patient |
| varices) and/or a | | evaluated by a |
| cirrhosis-like pattern | | hepatologist. |
| is seen on a CT | | |
| scan of the liver, the | | |
| possibility of NRH | | |
| should be considered. | | |

GI = gastrointestinal; LFT = liver function test; MBC = metastatic breast cancer; NRH = Nodular Regenerative Hyperplasia; TNF = tumor necrosis factor; ULN = upper limit of normal.

Left Ventricular Dysfunction

Patients treated with trastuzumab emtansine are at risk of developing left ventricular dysfunction. To date, significant cardiac events, including LVEF of <40%, have been observed (infrequently) in clinical trials of trastuzumab emtansine; therefore, symptomatic CHF is a potential risk.

Patients must meet specified LVEF requirements to be included in this study as described earlier.

Left ventricular function will be monitored by measurement of ejection fraction using ECHO) or MUGA scans.

Guidelines for patient monitoring and management of trastuzumab emtansine in patients who develop left ventricular dysfunction are provided in the following Table 22.

TABLE 22
Trastuzumab Emtansine Dose Modification
Guidelines for Neuropathy

| Event | Action to be taken |
|---|---|
| Grade ≥3 peripheral | Withhold trastuzumab emtansine until |
| neuropathy | recovery to Grade ≤2. Following recovery, |
| | resume trastuzumab emtansine at the same |
| | dose level or with one dose level reduction, |
| | at the investigator's discretion. Discontinue |
| | trastuzumab emtansine if the event has not |
| | resolved to Grade ≤2 within 42 days after |
| | the last dose received. |

Infusion Related Reactions and Hypersensitivity Reactions

Infusion-related reactions (IRRs) and hypersensitivity reactions have been reported with administration of trastuzumab emtansine. Despite the different pathophysiology of IRRs (reactions involving cytokine release) and hypersensitivity (allergic) reactions, the clinical manifestations are the same. In general, IRRs are expected to be more frequent and severe with the first infusion and to decrease in number and severity over time. The severity of true hypersensitivity reactions would be expected to increase with subsequent infusions.

IRRs, characterized by one or more of the following symptoms—flushing, chills, pyrexia, dyspnea, hypotension, wheezing, bronchospasm, and tachycardia—have been reported in clinical trials of trastuzumab emtansine. In general, these symptoms were not severe. In most patients, these reactions resolved over the course of several hours to a day after the infusion was terminated.

Hypersensitivity reactions, including serious anaphylactic-like reactions, have been observed in clinical trials of trastuzumab emtansine.

Patients with a history of intolerance to trastuzumab will be excluded from this study. Administration of trastuzumab emtansine will be performed in a setting with access to emergency facilities and staff who are trained to monitor and respond to medical emergencies. Patients should be closely monitored for IRRs during and after each infusion of trastuzumab emtansine.

TABLE 23
Management Guidelines for Trastuzumab Emtansine Infusion-Related Reactions
(Caused by Cytokine Release) or Hypersensitivity (Allergic) Reaction

| Event | Action to be taken |
|---|---|
| Grade 2 reaction | Decrease trastuzumab emtansine infusion rate or interrupt |
| | infusion. Administer supportive care with oxygen, 3-agonists, |
| | antihistamines, antipyretics, or corticosteroids, as appropriate, at |
| | the investigator's discretion. Monitor patient until complete |
| | resolution of symptoms. |
| | May continue trastuzumab emtansine at the same dose level at the |
| | investigator's discretion. In the event of a true hypersensitivity |
| | reaction (in which severity of reaction increases with subsequent |
| | infusions), discontinue trastuzumab emtansine. |
| | Premedication for infusion reactions (e.g., antihistamines such as |
| | diphenhydramine or corticosteroids) may be given at the |
| | investigator's discretion. |
| Grade 3 reaction | Stop trastuzumab emtansine infusion. Administer supportive care |
| | with oxygen, 3-agonists, antihistamines, antipyretics, or |
| | corticosteroids, as appropriate, at the investigator's discretion. |
| | Monitor patient until complete resolution of symptoms. |
| | May continue trastuzumab emtansine at the same dose level at |
| | the investigator's discretion. In the event of a true hypersensitivity |
| | reaction (in which severity of reaction increases with subsequent |
| | infusions), discontinue trastuzumab emtansine. |
| | Premedication for infusion reactions (e.g., antihistamines such as |
| | diphenhydramine or corticosteroids) may be given at the |
| | investigator's discretion. |
| Grade 4 | Stop trastuzumab emtansine infusion. Administer supportive care |
| | with oxygen, β-agonists, antihistamines, antipyretics, or |
| | corticosteroids, as appropriate, at the investigator's discretion. |
| | Monitor patient until complete resolution of symptoms. |
| | Discontinue trastuzumab emtansine. |

Hematologic Toxicity

Thrombocytopenia has been reported in patients in clinical trials of trastuzumab emtansine. The majority of these patients had Grade 1 or 2 events (platelet count ≥50,000 /μL), with the nadir occurring by Day 8 and generally improving to Grade 0 or 1 (platelet count ≥75,000 /μL) by the next scheduled dose (i.e., within 3 weeks). In clinical trials, the incidence and severity of thrombocytopenia were higher in Asian patients.

Cases of bleeding events with a fatal outcome have been observed. Severe cases of hemorrhagic events, including central nervous system hemorrhage, have been reported in clinical trials of trastuzumab emtansine; these events were independent of ethnicity. In some of the observed cases, the patients were also receiving anti-coagulation therapy.

Declines in other hematopoietic lineages, for example, leukopenia, neutropenia, and anemia, were less frequent than that observed for platelets.

Patients must meet specified hematologic laboratory test requirements to be included in this study, as described earlier.

Hematologic laboratory parameters will be monitored according to a prescribed schedule of assessments. Patients on anticoagulant or antiplatelet treatment should be monitored closely.

Guidelines for management of trastuzumab emtansine in patients who develop hematologic toxicity are provided in the following Table 24.

TABLE 24
Trastuzumab Emtansine Dose Modification
Guidelines for Hematological Toxicity

| Event | Action to Be Taken |
|---|---|
| Grade 2 thrombocytopenia | Assess platelet counts weekly or as medically |
| ( [symbol] L) | indicated until recovery. Withhold study treatment |
| | until Grade δ 1. Resume treatment without dose |
| | reduction. |
| Grade 3 thrombocytopenia | Withhold trastuzumab emtansine until recovery to |
| (25,000 to <50,000/ [symbol] L) | Grade δ 1 (ε 75,000/ [symbol] L). Following recovery, |
| | resume trastuzumab emtansine at the same dose |
| | level. Discontinue trastuzumab emtansine if the |
| | event has not resolved to Grade δ 1 within 42 |
| | days after the last dose received. |
| Grade 4 thrombocytopenia | Withhold trastuzumab emtansine until recovery to |
| (<25,000/ [symbol] L) at any time | Grade δ 1 (ε 75,000/ [symbol] L). Following recovery, |
| | resume trastuzumab emtansine with one dose level |
| | reduction. Discontinue trastuzumab emtansine if the |
| | event has not resolved to Grade δ 1 within 42 |
| | days after the last dose received. |
| Grade ε 3 hematologic | Withhold trastuzumab emtansine until recovery to |
| toxicity other than | Grade δ 2. Following recovery, resume trastuzumab |
| thrombocytopenia | emtansine at the same dose level. Discontinue |
| | trastuzumab emtansine if the event has not resolved |
| | to Grade δ 2 within 42 days after the last dose |
| | received. |

Neurotoxicity

Peripheral neuropathy, mainly Grade 1 and predominantly sensory, has been reported in clinical trials of trastuzumab emtansine.

Patients with Grade≥3 peripheral neuropathy will be excluded from this study.

Patients will be clinically monitored on an ongoing basis for signs or symptoms of peripheral neuropathy.

Guidelines for management of trastuzumab emtansine in patients who develop peripheral neuropathy are provided in Table 25 below.

TABLE 25
Trastuzumab emtansine guidelines for management of peripheral neuropathy

| Event | Action to Be Taken |
|---|---|
| Grade ≥3 peripheral neuropathy | Withhold trastuzumab emtansine until recovery to |
| | Grade ≤2. Following recovery, resume |
| | trastuzumab emtansine at the same dose level or |
| | with one dose level reduction, at the |
| | investigator's discretion. Discontinue trastuzumab |
| | emtansine if the event has not resolved to Grade ≤2 |
| | within 42 days after the last dose received |

Extravasation

In trastuzumab emtansine clinical studies, reactions secondary to extravasation have been observed. These reactions were usually mild and consisted of erythema, tenderness, skin irritation, pain, or swelling at the infusion site. These reactions have been observed more frequently within 24 hours of infusion.

The infusion site will be closely monitored for possible subcutaneous infiltration during drug administration. Specific treatment for trastuzumab emtansine extravasation is unknown at this time. Patients should be managed symptomatically per local institutional guidelines.

Risks Associated with Atezolizumab

The PD-L1/PD-1 pathway is involved in peripheral tolerance; therefore, such atezolizumab therapy may increase the risk of immune-mediated adverse events, specifically the induction or enhancement of autoimmune conditions. Adverse events with potentially immune-mediated causes, including rash, hypothyroidism, hepatitis or transaminitis, colitis, myositis, and myasthenia gravis have been observed in the Study PCD4989g.

Although most immune-mediated adverse events observed with immunomodulatory agents have been mild and self-limiting, such events should be recognized early and treated promptly to avoid potential major complications.

Management of Adverse Effects

Patients should be assessed for toxicity prior to each dose; dosing will occur only if the clinical assessment and laboratory test values are acceptable as described in the protocol. Dose delays, reductions and management guidelines are designed to ensure patient safety.

Dose Modification

Reasons for dose modifications or delays, the supportive measures taken, and the outcomes will be documented in the patient's chart and recorded on the eCRF. The severity of adverse events will be graded according to the NCI CTCAE v4.0.

When several toxicities with different grades of severity occur at the same time, the dose modifications should be according to the highest grade observed.

If, in the opinion of the investigator, a toxicity is considered to be attributable solely to one component of the study treatment (i.e., trastuzumab emtansine, atezolizumab or placebo and the dose of that component is delayed or modified in accordance with the guidelines below, the other component may be administered if there is no contraindication. If trastuzumab emtansine is discontinued for toxicity, then atezolizumab or placebo must also be discontinued.

When study treatment is temporarily interrupted because of toxicity caused by trastuzumab emtansine or atezolizumab/placebo, the treatment cycles will be restarted such that the atezolizumab/placebo/+trastuzumab emtansine infusions remain synchronized.

Dose interruptions for reason(s) other than adverse events, such as surgical procedures, may be allowed with Medical Monitor approval. The acceptable length of interruption will depend on agreement between the investigator and the Medical Monitor.

There will be no dose reduction for atezolizumab or placebo in this study. Patients may temporarily suspend study treatment if they experience toxicity that is considered related to atezolizumab or placebo and requires a dose to be withheld. If atezolizumab is withheld because of related adverse events for >42 days beyond when the next dose would have been given, then the patient will be discontinued from atezolizumab or placebo treatment and will be followed for safety and efficacy. If, in the judgment of the investigator, the patient is likely to derive clinical benefit from resuming atezolizumab or placebo after a hold >42 days, study drug may be restarted with the approval of the Medical Monitor.

If patients must be tapered off steroids for the treatment of adverse events related to atezolizumab or placebo, study treatment may be withheld for ≥42 days until steroids are discontinued or reduced to prednisone dose (or dose equivalent)≤10 mg/day. The acceptable length of interruption will depend on agreement between the investigator and the Medical Monitor.

If significant trastuzumab emtansine-related toxicities have not recovered to Grade 1 or baseline, the next scheduled dose may be delayed for ≤42 days after the last dose was received. “Significant” and “related” will be based on the judgment of the investigator (in consultation with the Sponsor's Medical Monitor or designee when appropriate). For example, alopecia even if considered related to trastuzumab emtansine would most likely not be considered to be significant. Fatigue may or may not be considered either related or significant. In general, when the significant related toxicity (or any other toxicity that the investigator chooses to delay dosing for) resolves to Grade 1 or baseline, the patient may resume trastuzumab emtansine if the delay is not >42 days from the last dose received.

Patients should be re-evaluated weekly during the delay, whenever possible. If dosing resumes, the patient may receive trastuzumab emtansine either at the same dose level as before or at one lower dose level (Table 16), at the discretion of the investigator. Subsequent cycles should remain q3w, and patients should be assessed for toxicity. If a patient requires a dose reduction, dosing will be reduced by one dose level as per Table 16.

A maximum of two dose reductions is allowed for trastuzumab emtansine. No dose re-escalation is permitted. A patient treated with 2.4 mg/kg of trastuzumab emtansine who develops an Adverse Event requiring a dose reduction must discontinue study treatment and will be followed for safety, disease progression and survival.

Patients who experience a Grade 3 or 4 hematologic events, other than thrombocytopenia, should be checked at least weekly for recovery. If values do not recover to baseline or Grade≤1 within 42 days from the last dose received, the patient will be discontinued from study treatment and will be followed for safety, disease progression, and survival.

Management of Patients Who have Trastuzumab Emtansine Related Specific Adverse Effects

Patients without significant cardiac history and with a baseline LVEF of ≥50% as determined by ECHO or MUGA scan are eligible for study participation. Cardiac monitoring (ECHO/MUGA) will be performed in all patients enrolled in the Study. Assessments will occur during the screening period, and on Day 15-21 of Cycle 1, and every fourth cycle thereafter. ECHO or MUGA will be performed following study treatment discontinuation only if the most recent follow-up ECHO/MUGA was performed ≥28 days after last study treatment administration or if no post-treatment evaluation was performed.

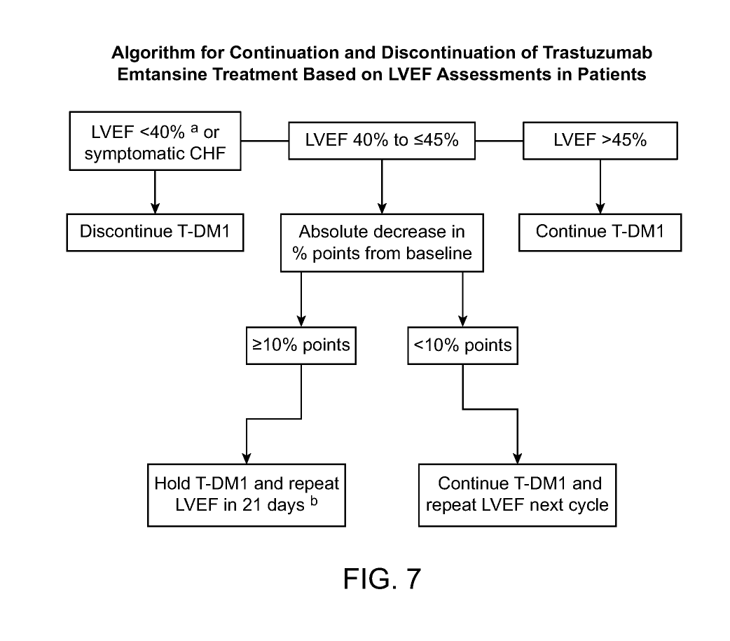

FIG. 7 summarizes the management of trastuzumab emtansine on the basis of LVEF measurements and changes in LVEF from baseline in patients. If an investigator is concerned that an adverse event may be related to cardiac dysfunction, an additional LVEF measurement may be performed. Trastuzumab emtansine will be discontinued in any patient who develops symptomatic CHF. CHF should be treated and monitored according to standard medical practice.

The decision to stop or continue trastuzumab emtansine treatment should be on the basis of the algorithm shown in FIG. 7. Trastuzumab emtansine must be discontinued in all patients for whom a confirmed decrease of LVEF to <40% is documented (with a confirmation assessment carried out within 21 days). For patients whose LVEF decreases to values of 40%-45% with an absolute decrease in LVEF of ≥10% points from baseline, trastuzumab emtansine dose should be held. For these patients, the LVEF measurement should be repeated within 21 days, and trastuzumab emtansine treatment should be discontinued if the LVEF has not recovered to within a 10% absolute difference below baseline. If clinically significant cardiac dysfunction or cardiac failure develops or persists or if significant medical management is required to maintain LVEF, the patient should be discontinued from all study treatment.

Hematological Toxicities

See Table 24 for trastuzumab emtansine dose modification guidelines for hematological toxicities, including thrombocytopenia.

Neuropathy

See Table 25 for trastuzumab emtansine dose modification guidelines for neuropathy.

Efficacy Analyses

Primary Efficacy Endpoint

The primary efficacy endpoint for this study is PFS based on investigator tumor assessment. The intention-to-treat (ITT) population is the primary analysis population for the primary efficacy endpoint and includes all patients who are randomized to the study, whether or not they receive any study medication.

PFS is defined as the time from randomization to first documented disease progression as determined by the investigator using RECIST 1.1 or death from any cause, whichever occurs earlier. The first documented disease progression will be used in the main analysis of the primary efficacy endpoint of PFS. Data for patients without disease progression or death from any cause as of the data cut-off date will be censored at the time of the last tumor assessment with an outcome other than “unevaluable” (or, if no tumor assessment was performed after the baseline visit, at the time of randomization plus 1 day). Data from patients who are lost to follow-up will be included in the analysis as censored observations on the date of the last tumor assessment that the patient was known to be progression-free. When disease progression or death occurs after two or more consecutive missed (or “unevaluable”) tumor assessments, these events will not be counted; rather, the patient will be censored at the patient's last tumor assessment prior to the first missing (or “unevaluable”) assessment. If disease progression or death occurs after one missed (or “unevaluable”) tumor assessment, the event will be counted at the respective event date.

The Kaplan-Meier method will be used to estimate median PFS for each treatment arm. The 2-sided log-rank test, stratified by world region (Western Europe vs U.S. vs Rest of World) and PD-L1 status (IC 0 vs IC 1-3) will be used to compare PFS between the treatment arms. The unstratified log-rank test result will also be provided. The stratified Cox regression model will be used to estimate the HR and to calculate the 95% CI of the HR.

A group sequential design will be used for testing the primary efficacy endpoint PFS to account for the conduct of interim analyses. An alpha spending using a gamma function with parameter −8 will be utilized to control the overall Type I error rate. The interim PFS analysis will be conducted when approximately 60 investigator-assessed PFS events (66.7% information fraction) have been observed and is anticipated to occur approximately 13 months from FPI. Key design characteristics of the interim PFS analysis are detailed in Table 26.

TABLE 26
PFS Interim Analysis Design Characteristics
using Gamma (-8) Alpha Spending Function

| Number of | Information | Cumulative | Crossing | Crossing Boundary in |
|---|---|---|---|---|
| PFS Events | Fraction | Alpha Spent | Boundary in HR | P-Value |
| 60 | 66.7% | 0.004 | HR ≤ 0.468 | p ≤ 0.002 |

HR = hazard ratio; PFS = progression-free survival.

The sponsor may decide to consider adding an additional interim analysis including efficacy data which will be pre-specified in the SAP as appropriate.

The interim analyses will be conducted by an iDMC with the support of an independent Data Coordinating Center (iDCC). Interactions between the iDMC and the sponsor will follow the iDMC charter. The decision to conduct the interim analysis, rationale, timing, and statistical details will be documented in the SAP. Additional interim analyses may be conducted if requested by health authorities. The final PFS analysis (will be performed when approximately 90 investigator-assessed PFS events have been observed and is anticipated to occur approximately 17 to 20 months from FPI, depending on PFS HR assumptions.

Several sensitivity analyses will be performed to assess the robustness of the primary efficacy analysis, see the SAP for details.

In order to assess the consistency of treatment benefit with respect to the primary efficacy endpoint PFS across important subgroups, forest plots (including estimated HRs) will be provided, including, but not limited, to the following variables: race, age, sex, world region, baseline PD-L1 expression, ECOG status and hormone receptor status. A multivariate Cox regression analysis will be performed on the primary efficacy endpoint of investigator-assessed PFS controlling for important baseline characteristics.

Secondary Efficacy Endpoints

The ITT population will be the analysis population used for evaluation of the secondary efficacy endpoints.

Overall Survival

OS is defined as the time from randomization to death from any cause. Patients who are alive as of the data cut-off date of the analysis will be censored at the last known date they were alive. Patients with no post-baseline information will be censored at the date of randomization plus 1 day. Methods for data analysis are analogous to those described for the primary efficacy endpoint.

Objective Response Rate

Objective response, defined as a CR or PR, will be determined by investigator tumor assessment using RECIST 1.1. Patients without a post-baseline tumor assessment will be considered non-responders. Objective responses must be confirmed at least 28 days after the initial documentation of response. An estimate of the ORR and its 95% CI (Blyth-Still-Casella) will be calculated for each treatment arm. The Cochran-Mantel-Haenszel Chi-squared test will be used to compare response rates between treatment arms. An unstratified Chi-squared test will also be provided. Finally, the difference in response rates between treatment arms will be computed with 95% CIs, using the normal approximation to the binomial distribution.

Duration of Response

DOR is defined as the time from first occurrence of a documented objective response (PR or CR) to disease progression, as determined by investigator tumor assessment using RECIST 1.1, or death from any cause, whichever occurs first. The analysis methods are similar to those described for the primary efficacy endpoint PFS. The limitations of this responder analysis are acknowledged.

PFS in Subgroups of Patients Defined as PD-L1Dx+ and PD-L1Dx.

The analysis methods are similar to those described for the primary efficacy endpoint.

Exploratory Efficacy Endpoints

The exploratory efficacy endpoints will be evaluated at time of primary efficacy analysis. The ITT population will be the analysis population used for evaluation of the exploratory efficacy endpoints.

PFS Assessed in the PD-L1 Selected Subgroup

The analysis methods are similar to those described for the primary efficacy endpoint

PFS Assessed using Immune-Modified RECIST

PFS is defined as the time from randomization to first occurrence of disease progression as determined by investigator assessment using immune-modified RECIST or death from any cause, whichever occurs earlier. Only patients who are clinically eligible for treatment beyond disease progression will be included in this analysis. The analysis methods are similar to those described for the primary efficacy endpoint.

Objective Response Rate Based on Immune Modified RECIST

Objective response, defined as a complete response (CR) or partial response (PR), will be determined by investigator tumor assessment using immune-modified RECIST. Patients without a post-baseline tumor assessment will be considered non-responders. Objective responses must be confirmed at least 28 days after the initial documentation of response. An estimate of the ORR and its 95% CI (Blyth-Still-Casella) will be calculated for each treatment arm. The stratified Cochran-Mantel-Haenszel Chi-squared test will be used to compare response rates between treatment arms. An unstratified Chi-squared test will also be provided. Finally, the difference in response rates between treatment arms will be computed with 95% CIs, using the normal approximation to the binomial distribution.

Duration of Response

DOR is defined as the time from first occurrence of a documented objective response (PR or CR) to disease progression, as determined by investigator tumor assessment using immune-modified RECIST, or death from any cause, whichever occurs first. The analysis methods are similar to those described for the primary efficacy endpoint PFS.

1-Year Survival Rate

Kaplan-Meier methodology will be used to estimate 1-year survival rates and 95% CIs for each treatment arm. Also, differences in 1-year survival rates between treatment arms will be calculated together with 95% CIs.

Safety Analyses

The safety analysis population will include all randomized patients who received at least one full or partial dose of study drug. Safety analyses will be performed based on the treatment the patient actually received.

Study Drug Exposure

The number of patients who experience any dose modification (including dose delay, dose reduction and dose interruption), or dose discontinuation, and reasons for study treatment discontinuation will be summarized for each of the treatment arm regimens. In addition, the number of patients that discontinue from trastuzumab emtansine-containing and/or atezolizumab-containing treatment because of toxicity and/or receive other non-protocol anti-cancer therapy will be summarized.

Descriptive statistics will be presented for total cumulative dose, number of cycles, dose intensity, infusion time by cycle, and weeks of exposure for trastuzumab emtansine, and atezolizumab.

Adverse Events

Verbatim descriptions of AEs will be mapped to Medical Dictionary for Regulatory Activities (MedDRA) thesaurus terms and graded according to the NCI CTCAE v4.0. The following events occurring on or after the first dose of study drug (i.e., treatment-emergent AEs) will be summarized by NCI CTCAE grade:
All AEs
SAEs
AEs leading to death
AEs leading to study drug discontinuation
AEs leading to dose reduction

For events of varying severity, the highest grade will be used in the summaries. Deaths and causes of death will be summarized. Selected AEs will be summarized by NCI CTCAE grade for each treatment arm based on pre-specified category definitions, including (but not limited to) hepatotoxicity, cardiac dysfunction, and thrombocytopenia. In addition, AEs occurring within 1 day (24 hours) of the first dose of each treatment cycle will be summarized to help characterize potential infusion-related reactions. Additional analyses may be performed as indicated.

Laboratory Data

For laboratory parameters, descriptive summary tables of change from baseline over time based on System International units will be produced. Summary tables for the shifts in NCI CTCAE v4.0 grades from baseline to the worst grade observed during treatment will be presented.

Pharmacokinetic Analyses

The PK analyses will include patients with at least one post-dose PK assessment

Individual serum atezolizumab, trastuzumab emtansine, total trastuzumab levels and plasma DM1 concentrations versus time will be tabulated and summarized by treatment arm and study visit day. Descriptive statistics will include mean, medians range, standard deviation, coefficient of variation (CV %), geometric mean, and geometric mean coefficient of variation (CVb %) as appropriate.

Additional PK and PD analyses will be conducted as appropriate.

Immunogenicity Analyses

The immunogenicity analyses will include patients with at least one predose and one post-dose ATA assessment, with patients grouped according to treatment received. The numbers and proportions of ATA-positive patients and ATA-negative patients during both the treatment and follow-up periods will be summarized by treatment group. Patients are considered to be ATA positive if they are ATA negative at baseline but develop an ATA response following study drug administration (treatment-induced ATA response), or if they are ATA positive at baseline and the titer of one or more post-baseline samples is at least 4-fold greater (i.e., ≥0.60 titer units) than the titer of the baseline sample (treatment-enhanced ATA response). Patients are considered to be ATA negative if they are ATA negative at baseline and all post-baseline samples are negative, or if they are ATA positive at baseline but do not have any post-baseline samples with a titer that is at least 4-fold greater than the titer of the baseline sample (treatment unaffected).

The impact of ATA on PK, efficacy and safety may be explored as appropriate.

Biomarker Analyses

Descriptive statistics will be utilized for the analysis and reporting of the exploratory biomarker objectives. This may include appropriate multivariate analyses.

### REFERENCES

Adams S, Diamond J, Hamilton E, et al. Safety and clinical activity of atezolizumab (anti-PDL1) in combination with nab-paclitaxel in patients with metastatic triple-negative breast cancer [abstract]. Cancer Res 2015; 76 (Suppl 4).
Andrulis I, Bull S, Blackstein M, et al. neu/erbB-2 amplification identifies a poor-prognosis group of women with node-negative breast cancer. Toronto Breast Cancer Study Group. J Clin Oncol 1998; 16:1340-9.
Baselga J, Javier Cortes J, Kim S-B, et al. Pertuzumab plus trastuzumab plus docetaxel for metastatic breast cancer. N Engl J Med 2012; 366:109-19.
Baselga J, Lewis Phillips G D, Verma S, et al. Relationship between tumor biomarkers and efficacy in EMILIA, a phase III study of trastuzumab emtansine in HER2-positive metastatic breast cancer. Clin Cancer Res, 2016.
Baselga J, Javier Cortes J, Im S A, et al. Biomarker analyses in CLEOPATRA: a phase III, placebo-controlled study of pertuzumab in human epidermal growth factor receptor 2-positive, first-line metastatic breast cancer. J Clin Oncol. 2014; 20:375361
Bianchini G, Pusztai L, Pienkowski T et al. Immune modulation of pathologic complete response after neoadjuvant HER2-directed therapies in the NeoSphere trial. Ann Oncol. 2015; 26:2429-36.
Blank C, Mackensen A. Contribution of the PD-L1/PD-1 pathway to T-cell exhaustion: an update on implications for chronic infections and tumor evasion. Cancer Immunol Immunother 2007; 56:739-45.
Blank C, Gajewski T F, Mackensen A. Interaction of PD-L1 on tumor cells with PD-1 on tumor-specific T cells as a mechanism of immune evasion: implications for tumor immunotherapy. Cancer Immunol Immunother 2005; 54:307 14.
Butte M J, Keir M E, Phamduy T B. Programmed death-1 ligand 1 interacts specifically with the B7-1 costimulatory molecule to inhibit T cell responses. Immunity 2007; 27: 111-22.
Cardoso F, Costa A, Norton L, Senkus E, Aapro M, André F, et al. ESO-ESMO 2nd international consensus guidelines for advanced breast cancer (ABC2). Ann Oncol. 2014; 25:1871-88
Chen G, Gaupta R, Petrik R S, et al. A feasibility study of cyclophosphamide, trastuzumab, and an allogeneic GM-CSF-secreting breast tumor vaccine for HER2+ metastatic breast cancer. Cancer Immuno Res 2014; 2:949-61.
Diéras V, Miles D, Verma S, et al. Trastuzumab Emtansine (T-DM1) Improves Overall Survival Versus Capecitabine Plus Lapatinib in Patients With Previously Treated HER2-Positive Advanced Breast Cancer: Final Results From the Phase 3 EMILIA Study. San Antonio Breast Cancer Symposium, Dec. 8-12, 2015.
Di Giacomo A M, Biagioli M, Maio M. The emerging toxicity profiles of anti-CTLA-4 antibodies across clinical indications. Semin Oncol 2010; 37:499-507.
Eisenhauer E A, Therasse P, Bogaerts J et al. New response evaluation criteria in solid tumours: revised RECIST guideline (version 1.1). Eur J Cancer. 2009; 2:228-47
Emens L A, Braiteh F S, Cassier P., Inhibition of PD-L1 by MPDL3280A leads to clinical activity in patients with metastatic triple-negative breast cancer. Presented at: 2015 AACR Annual Meeting; April 18-22; Philadelphia, Pa. Abstract 6317
Gettinger S N, Kowanetz M. Molecular correlates of PD-L1 status and predictive biomarkers in patients with non-small cell lung cancer (NSCLC) treated with the anti-PDL1 antibody MPDL3280A. Mini oral abstract session presented at the International Association for the Study of Lung Cancer 15th World Conference; 27-30 Oct. 2013; Sydney, Australia. Session MO19.09.
Herbst R S, Soria J C, Kowanetz M, et al. Predictive correlates of response to the anti-PD-1 antibody MPDL3280 in cancer patients. Nature 2014; 515:563-7.
International Agency for Research on Cancer. Globocan 2012; [resource on the internet]. Available from: http://www-dep.iarc.fr
Keir M E, Butte M J, Freeman G J, et al. PD-1 and its ligands in tolerance and immunity. Annual Rev Immunol 2008; 26:677 704.
Krop I, Kim S-B, Gonzalez-Martin A et al. Trastuzumab emtansine versus treatment of physician's choice for pretreated HER2-positive advanced breast cancer (TH3RESA): a randomised, open-label, phase 3 trial. Lancet Oncology. 2014; 15:689-699
Marty M, Cognetti F, Maraninchi D et al. Randomized phase II trial of the efficacy and safety of trastuzumab combined with docetaxel in patients with human epidermal growth factor receptor 2-positive metastatic breast cancer administered as first-line treatment: the M77001 study group. J Clin Oncol. 2005; 19:4265-74.
Müller P, Kreuzaler P, Khan T, et al. Trastuzumab emtansine (T-DM1) renders HER2+ breast cancer highly susceptible to CTLA-4/PD-1 blockade. Sci Transl Medi. 2015; 7:315
National Comprehensive Cancer Network. NCCN Clinical Practice Guidelines in Oncology: invasive breast cancer, version 3.2014. Available at http://www.nccn.org/professionals/physician_gls/pdf/breast.pdf. 2014a.
Owens M A, Horten B C, Da Silva M M. HER2 amplification ratios by fluorescence in situ hybridization and correlation with immunohistochemistry in a cohort of 6556 breast cancer tissues. Clin Breast Cancer. 2004; 5:63⁻9.
Pauletti G, Dandekar S, Rong H, et al. Assessment of methods for tissue-based detection of the HER-2/neu alteration in human breast cancer: a direct comparison of fluorescence in situ hybridization and immunohistochemistry. J Clin Oncol 2000; 18:3651-64.
Reese D M, Slamon D J. HER-2/neu signal transduction in human breast and ovarian cancer. Stem Cells. 1997; 15:1-8.
Rosenberg J E, Hoffman-Censits J, Powles T et al. Atezolizumab in patients with locally advanced and metastatic urothelial carcinoma who have progressed following treatment with platinum-based chemotherapy: a single-arm, multicentre, phase 2 trial. Lancet. 2016 Mar. 4.
Rubin I, Yarden Y. The basic biology of HER2. Ann Oncol 2001; 12(Suppl 1):S-8.
Slamon D J, Leyland-Jones B, Shak S, et al. Use of chemotherapy plus a monoclonal antibody against HER2 for metastatic breast cancer that overexpresses HER2. N Engl J Med 2001; 344:783-92.
Slamon D J, Clark G M, Wong S G, et al. Human breast cancer: correlation of relapse and survival with amplification of the HER-2/neu oncogene. Science 1987; 235:177-82.
Spigel D R, Chaft J E, Gettinger S et al. Clinical activity and safety from a phase II study (FIR) of MPDL3280A (anti-PDL1) in PD-L1 selected patients with non-small cell lung cancer (NSCLC). Proceedings of the 51st ASCO Annual Meeting:2015 Jun. 3-7: Chicago. Available from: http://meetinglibrary.asco.org/content/108536?media=vm
Spira A, Park K, Mazieres J et al. Efficacy, safety and predictive biomarker results from a randomized phase II study comparing MPDL3280A vs docetaxel in patients with advanced NSCLC (POPLAR). Proceedings of the 51st ASCO Annual Meeting:2015 Jun. 3-7: Chicago. Available from:
Toikkanen S, Helin H, Isola J, et al. Prognostic significance of HER-2 oncoprotein expression in breast cancer: a 30-year follow-up. J Clin Oncol 1992; 10:1044-8.
Verma S, Miles D, Gianni L, et al. Trastuzumab emtansine for HER2-positive advanced breast cancer. N Eng J Med 2012; 367:1783-91.
Wildiers H, Sung-Bae K, Antonio Gonzalez M et al. Trastuzumab emtansine (T-DM1) improves overall survival versus treatment of physician's choice in patients with previously treated HER2-positive metastatic breast cancer: final overall survival results from the phase 3 TH3RESA study. San Antonio Breast Cancer Symposium, Dec. 8-12, 2015.
Wolchok J D, Hoos A, O'Day S et al. Guidelines for the evaluation of immune therapy activity in solid tumors: immune-related response criteria. Clin Cancer Res. 2009; 23:7412-20.
Wolff A C, Hammond M E, Hicks D G et al. Recommendations for human epidermal growth factor receptor 2 testing in breast cancer: American Society of Clinical Oncology/College of American Pathologists clinical practice guideline update. J Clin Oncol 2013; 31:3997-4013
Yang J, Riella L V, Chock S. The novel costimulatory programmed death ligand 1/B7.1 pathway is functional in inhibiting alloimmune responses in vivo. J Immunol 2011; 187:1113-9.
Zhang H, Zhang S, Wang Y, et al. Re-evaluation of HER2 status in 1 501 invasive breast cancers according to the 2013 American Society of Clinical Oncology/College of American Pathology guidelines. 2015; 44:42⁻7.

TABLE OF SEQUENCES

| SEQ ID | |
|---|---|
| NO: | Sequence |
| SEQ ID | Trastuzumab light chain amino acid |
| NO: 1 | sequence (FIG. 8A) |
| SEQ ID | Trastuzumaqb heavy chain amino acid |
| NO: 2 | sequence (FIG. 8B) |
| SEQ ID | EVQLVESGGGLVQPGGSLRLSCAASGFTFSDSWIHWVRQ |
| NO: 3 | APGKGLEWVAWISPYGGSTYYADSVKGRFTISADTSKNT |
| | AYLQMNSLRAEDTAVYYCARRHWPGGFDYWGQGTLVT |
| | VSS |
| SEQ ID | DIQMTQSPSSLSASVGDRVTITCRASQDVSTAVAWYQQK |
| NO: 4 | PGKAPKLLIYSASFLYSGVPSRFSGSGSGTDFTLTISSL |
| | QPEDFATYYCQQYLYHPATFGQGTKVEIKR |
| SEQ ID | GFTFSX₁SWIH |
| NO: 5 | |
| SEQ ID | AWIX₂PYGGSX₃YYADSVKG |
| NO: 6 | |
| SEQ ID | RHWPGGFDY |
| NO: 7 | |
| SEQ ID | GFTFSDSWIH |
| NO: 8 | |
| SEQ ID | AWISPYGGSTYYADSVKG |
| NO: 9 | |
| SEQ ID | RHWPGGFDY |
| NO: 10 | |
| SEQ ID | Intentionally left blank |
| NO: 11 | |
| SEQ ID | RASQX₄X₅X₆TX₇X₈A |
| NO: 12 | |
| SEQ ID | SASX₉LX₁₀S |
| NO: 13 | |
| SEQ ID | QQX₁₁X₁₂X₁₃X₁₄PX₁₅T |
| NO: 14 | |
| SEQ ID | RASQDVSTAVA |
| NO: 15 | |
| SEQ ID | SASFLYS |
| NO: 16 | |
| SEQ ID | QQYLYHPAT |
| NO: 17 | |
| SEQ ID | Pertuzumab light chain amino acid |
| NO: 18 | sequence (FIG. 9A) |
| SEQ ID | Pertuzumab heavy chain amino acid |
| NO: 19 | sequence (FIG. 9B) |

We claim:

1. A method of treating HER2 positive breast cancer, the method comprising administering to a patient having said breast cancer a therapeutically effective amount of a programmed cell death protein 1 (PD-1) binding antagonist or a programmed death ligand 1 (PD-L1) binding antagonist in combination with trastuzumab and pertuzumab.

2. The method of claim 1, wherein the HER2 positive breast cancer is first line metastatic HER2 positive breast cancer.

3. The method of claim 1, wherein the HER2 positive breast cancer is operable or locally advanced HER2 positive breast cancer.

4. The method of claim 1, wherein the HER2 positive breast cancer is HER2 positive inflammatory early breast cancer.

5. The method of any one of claims 1-4 comprising administering a PD-1 antagonist.

6. The method of any one of claims 1-4 comprising administering a PD-L1 antagonist.

7. The method of claim 5, wherein the PD-1 antagonist is an anti-PD-1 antibody or an antigen-binding fragment thereof.

8. The method of claim 6, wherein the PD-L1 antagonist is an anti-PD-L1 antibody or an antigen-binding fragment thereof.

9. The method of claim 8, wherein the anti-PD-L1 antibody comprises:
(a) an HVR-H1 sequence of GFTFSDSWIH (SEQ ID NO:8);
(b) an HVR-H2 sequence of AWISPYGGSTYYADSVKG (SEQ ID NO:9);
(c) an HVR-H3 sequence of RHWPGGFDY (SEQ ID NO:10);
(d) an HVR-L1 sequence of RASQDVSTAVA (SEQ ID NO:15);
(e) an HVR-L2 sequence of SASFLYS, (SEQ ID NO:16); and
(f) an HVR-L3 sequence of QQYLYHPAT (SEQ ID NO:17).

10. The method of claim 8, wherein the anti-PD-L1 antibody comprises the heavy chain variable region of SEQ ID NO:3 and the light chain variable region of SEQ ID NO:4.

11. The method of claim 8, wherein the anti-PD-L1 antibody is atezolizumab.

12. The method of claim 11, wherein atezolizumab is administered by infusion at a dose of 1200 mg on the first day of treatment and every three weeks thereafter; trastuzumab is administered by infusion at a loading dose of 8 mg/kg on the first day of treatment and at a dose of 6 mg/kg every three weeks thereafter; and pertuzumab is administered by infusion at a loading dose of 840 mg on the first day of treatment and at a dose of 420 mg every three weeks thereafter.

13. The method of any of claim 11, wherein the treatment is given as neoadjuvant therapy.

14. The method of claim 13, wherein the method comprises administering atezolizumab in combination with trastuzumab and pertuzumab, and wherein atezolizumab is administered by infusion at a dose of 1200 mg on the first day of treatment and every three weeks thereafter; trastuzumab is administered by infusion at a loading dose of 8 mg/kg on the first day of treatment and at a dose of 6 mg/kg every three weeks thereafter; and pertuzumab is administered by infusion at a loading dose of 840 mg on the first day of treatment and at a dose of 420 mg every three weeks thereafter.

15. The method of claim 14, wherein atezolizumab is administered in combination with trastuzumab and pertuzumab every three weeks for two cycles, followed by administration of a therapeutic regimen comprising chemotherapy.

16. The method of claim 15, wherein the therapeutic regimen comprising chemotherapy comprises trastuzumab, pertuzumab, carboplatin and docetaxel.

17. The method of claim 16, wherein carboplatin is administered by infusion at a dose of 6 mg/ml·min every three weeks; docetaxel is administered by infusion at a dose of 75 mg/m² every three weeks; trastuzumab is administered by infusion at a dose of 6 mg/kg every three weeks; and pertuzumab is administered by infusion at a dose of 420 mg every three weeks.

18. The method of claim 16 or claim 17, wherein the therapeutic regimen comprising chemotherapy is administered for six cycles.

19. The method of claim 18, wherein after the six cycles of the therapeutic regimen comprising chemotherapy, the patient is subjected to definitive surgery.

20. The method of claim 19, wherein after definitive surgery, trastuzumab is administered to the patient.

21. The method of claim 19, wherein after definitive surgery, trastuzumab is administered to the patient by infusion at a dose of 6 mg/kg every three weeks.

22. The method of claim 19, wherein after definitive surgery, trastuzumab is administered to the patient by infusion at a dose of 6 mg/kg every three weeks for twelve cycles.

23. A method of treating HER2 positive breast cancer, the method comprising administering to a patient having said breast cancer a therapeutically effective amount of programmed cell death protein 1 (PD-1) binding antagonist or a programmed death ligand 1 (PD-L1) binding antagonist in combination with trastuzumab emtansine.

24. The method of claim 23, wherein the HER2 positive breast cancer is first line metastatic HER2 positive breast cancer.

25. The method of claim 23, wherein the HER2 positive breast cancer is first line metastatic HER2 positive breast cancer and the patient has received prior treatment with trastuzumab and a taxane.

26. The method of claim 23, wherein the HER2 positive breast cancer is operable or locally advanced HER2 positive breast cancer.

27. The method of claim 23, wherein the HER2 positive breast cancer is HER2 positive inflammatory early breast cancer.

28. The method of any one of claims 23-27 comprising administering a PD-1 antagonist.

29. The method of any one of claims 23-27 comprising administering a PD-L1 antagonist.

30. The method of claim 28, wherein the PD-1 antagonist is an anti-PD-1 antibody or an antigen-binding fragment thereof.

31. The method of claim 29, wherein the PD-L1 antagonist is an anti-PD-L1 antibody or an antigen-binding fragment thereof.

32. The method of claim 31, wherein the anti-PD-L1 antibody comprises:
(a) an HVR-H1 sequence of GFTFSDSWIH (SEQ ID NO:8);
(b) an HVR-H2 sequence of AWISPYGGSTYYADSVKG (SEQ ID NO:9);
(c) an HVR-H3 sequence of RHWPGGFDY (SEQ ID NO:10);
(d) an HVR-L1 sequence of RASQDVSTAVA (SEQ ID NO:15);
(e) an HVR-L2 sequence of SASFLYS, (SEQ ID NO:16); and
(f) an HVR-L3 sequence of QQYLYHPAT (SEQ ID NO:17).

33. The method of claim 31, wherein the anti-PD-L1 antibody comprises the heavy chain variable region of SEQ ID NO:3 and the light chain variable region of SEQ ID NO:4.

34. The method of claim 31, wherein the anti-PD-L1 antibody is atezolizumab.

35. The method of claim 34, wherein atezolizumab is administered by infusion at a dose of 1200 mg every three weeks and trastuzumab emtansine is administered by infusion at dose of 3.6 mg/kg every three weeks.

36. The method of any of claims 23-27, wherein the treatment is given as neoadjuvant therapy.

37. The method of claim 36, wherein the method comprises administering atezolizumab in combination with trastuzumab emtansine, and wherein atezolizumab is administered by infusion at a dose of 1200 mg every three weeks and trastuzumab emtansine is administered by infusion at dose of 3.6 mg/kg every three weeks.

38. The method of claim 37, wherein atezolizumab in combination with trastuzumab emtansine is administered every three weeks for two cycles, followed by administration of a therapeutic regimen comprising chemotherapy.

39. The method of claim 38, wherein the therapeutic regimen comprising chemotherapy comprises carboplatin, docetaxel, trastuzumab and pertuzumab.

40. The method of claim 39, wherein carboplatin is administered by infusion at a dose of 6 mg/ml·min every three weeks; docetaxel is administered by infusion at a dose of 75 mg/m² every three weeks; trastuzumab is administered by infusion at a loading dose of 8 mg/kg on the first day of treatment with trastuzumab, and at a dose of 6 mg/kg every three weeks thereafter; and pertuzumab is administered by infusion at a loading dose of 840 mg on the first day of treatment with pertuzumab, and at a dose of 420 mg every three weeks thereafter.

41. The method of claim 39 or claim 40, wherein the therapeutic regimen comprising chemotherapy is administered for six cycles.

42. The method of claim 41, wherein after the six cycles of the therapeutic regimen comprising chemotherapy, the patient is subjected to definitive surgery.

43. The method of claim 42, wherein after definitive surgery, trastuzumab is administered to the patient.

44. The method of claim 42, wherein after definitive surgery, trastuzumab is administered to the patient by infusion at a dose of 6 mg/kg every three weeks.

45. The method of claim 42, wherein after definitive surgery, trastuzumab is administered to the patient by infusion at a dose of 6 mg/kg every three weeks for twelve cycles.

46. Use of a therapeutically effective amount of a programmed cell death protein 1 (PD-1) binding antagonist or a programmed death ligand 1 (PD-L1) binding antagonist in the preparation of a medicament for the treatment of HER2 positive breast cancer in combination with trastuzumab and pertuzumab.

47. A pharmaceutical composition comprising a therapeutically effective amount of a programmed cell death protein 1 (PD-1) binding antagonist or a programmed death ligand 1 (PD-L1) binding antagonist for the treatment of HER2 positive breast cancer in combination with trastuzumab and pertuzumab.

48. Use of a therapeutically effective amount of a programmed cell death protein 1 (PD-1) binding antagonist or a programmed death ligand 1 (PD-L1) binding antagonist in the preparation of a medicament for the treatment of HER2 positive breast cancer in combination with trastuzumab emtansine.

49. A pharmaceutical composition comprising a therapeutically effective amount of a programmed cell death protein 1 (PD-1) binding antagonist or a programmed death ligand 1 (PD-L1) binding antagonist for the treatment of HER2 positive breast cancer in combination with trastuzumab emtansine.

50. The use of claim 46 or claim 48 or the pharmaceutical composition of claim 47 or claim 49, wherein the PD-L1 antagonist is an anti-PDL-1 antibody or an antigen-binding fragment thereof.

51. The use or pharmaceutical composition of claim 50, wherein the anti-PD-L1 antibody comprises:
(a) an HVR-H1 sequence of GFTFSDSWIH (SEQ ID NO:8);
(b) an HVR-H2 sequence of AWISPYGGSTYYADSVKG (SEQ ID NO:9);
(c) an HVR-H3 sequence of RHWPGGFDY (SEQ ID NO:10);
(d) an HVR-L1 sequence of RASQDVSTAVA (SEQ ID NO:15);
(e) an HVR-L2 sequence of SASFLYS, (SEQ ID NO:16); and
(f) an HVR-L3 sequence of QQYLYHPAT (SEQ ID NO:17).

52. The use or pharmaceutical composition of claim 50, wherein the anti-PD-L1 antibody is atezolizumab.


SEQUENCE LISTING (19 sequences)

SEQ ID NO: 1 - protein, 214 aa, Artificial Sequence
  source: /note=“Description of Artificial Sequence: Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, Artificial Sequence
  source: /note=“Description of Artificial Sequence: Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 118 aa, Artificial Sequence [sequence omitted]

SEQ ID NO: 4 - protein, 108 aa, Artificial Sequence
  source: /note=“Description of Artificial Sequence: Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 5 - protein, 10 aa, Artificial Sequence
  source: /note=“Description of Artificial Sequence: Synthetic peptide”
  VARIANT (6)..(6): /replace=“Gly”
  MISC_FEATURE (1)..(10): /note=“Variant residues given in the sequence have no preference with respect to those in the annotations for variant positions”
GFTFSDSWIH

SEQ ID NO: 6 - protein, 18 aa, Artificial Sequence
  source: /note=“Description of Artificial Sequence: Synthetic peptide”
  VARIANT (4)..(4): /replace=“Leu”
  VARIANT (10)..(10): /replace=“Ser”
  MISC_FEATURE (1)..(18): /note=“Variant residues given in the sequence have no preference with respect to those in the annotations for variant positions”
AWISPYGGSTYYADSVKG

SEQ ID NO: 7 - protein, 9 aa, Artificial Sequence
  source: /note=“Description of Artificial Sequence: Synthetic peptide”
RHWPGGFDY

SEQ ID NO: 8 - protein, 10 aa, Artificial Sequence
  source: /note=“Description of Artificial Sequence: Synthetic peptide”
GFTFSDSWIH

SEQ ID NO: 9 - protein, 18 aa, Artificial Sequence
  source: /note=“Description of Artificial Sequence: Synthetic peptide”
AWISPYGGSTYYADSVKG

SEQ ID NO: 10 - protein, 9 aa, Artificial Sequence
  source: /note=“Description of Artificial Sequence: Synthetic peptide”
RHWPGGFDY

SEQ ID NO: 11 - skipped

SEQ ID NO: 12 - protein, 11 aa, Artificial Sequence
  source: /note=“Description of Artificial Sequence: Synthetic peptide”
  VARIANT (5)..(5): /replace=“Val”
  VARIANT (6)..(6): /replace=“Ile”
  VARIANT (7)..(7): /replace=“Asn”
  VARIANT (9)..(9): /replace=“Phe”
  VARIANT (10)..(10): /replace=“Leu”
  MISC_FEATURE (1)..(11): /note=“Variant residues given in the sequence have no preference with respect to those in the annotations for variant positions”
RASQDVSTAVA

SEQ ID NO: 13 - protein, 7 aa, Artificial Sequence
  source: /note=“Description of Artificial Sequence: Synthetic peptide”
  VARIANT (4)..(4): /replace=“Thr”
  VARIANT (6)..(6): /replace=“Ala”
  MISC_FEATURE (1)..(7): /note=“Variant residues given in the sequence have no preference with respect to those in the annotations for variant positions”
SASFLYS

SEQ ID NO: 14 - protein, 9 aa, Artificial Sequence
  source: /note=“Description of Artificial Sequence: Synthetic peptide”
  VARIANT (3)..(3): /replace=“Gly” or “Phe” or “Ser”
  VARIANT (4)..(4): /replace=“Tyr” or “Phe” or “Trp”
  VARIANT (5)..(5): /replace=“Asn” or “Ala” or “Thr” or “Gly” or “Phe” or “Ile”
  VARIANT (6)..(6): /replace=“Val” or “Pro” or “Thr” or “Ile”
  VARIANT (8)..(8): /replace=“Trp” or “Arg” or “Pro” or “Thr”
  MISC_FEATURE (1)..(9): /note=“Variant residues given in the sequence have no preference with respect to those in the annotations for variant positions”
QQYLYHPAT

SEQ ID NO: 15 - protein, 11 aa, Artificial Sequence
  source: /note=“Description of Artificial Sequence: Synthetic peptide”
RASQDVSTAVA

SEQ ID NO: 16 - protein, 7 aa, Artificial Sequence
  source: /note=“Description of Artificial Sequence: Synthetic peptide”
SASFLYS

SEQ ID NO: 17 - protein, 9 aa, Artificial Sequence
  source: /note=“Description of Artificial Sequence: Synthetic peptide”
QQYLYHPAT

SEQ ID NO: 18 - protein, 214 aa, Artificial Sequence
  source: /note=“Description of Artificial Sequence: Synthetic polypeptide”
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 448 aa, Artificial Sequence
  source: /note=“Description of Artificial Sequence: Synthetic polypeptide”
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG